(19) United States
(12) Patent Application Publication
(10) Pub. No.: US 2007/0298026 A1
(43) Pub. Date: Dec. 27, 2007

(54) Methods for Identifying Treatment and Inducing Infertility Using Smc1-Beta

(75) Inventors: Rolf Jessberger, White Plains, NY (US); Ekaterina Revenkova, Fort Lee, NJ (US)

Correspondence Address: MARSHALL, GERSTEIN & BORUN LLP, 233 S. WACKER DRIVE, SUITE 6300, SEARS TOWER, CHICAGO, IL, 60606, US

(73) Assignee: MOUNT SINAI SCHOOL OF MEDICINE Office of Industrial Liaison, New York, NY (US)

(21) Appl. No.: 10/570,047

(22) PCT Filed: Aug. 30, 2004
(86) PCT No.: PCT/US04/28240

§ 371 Date: Apr. 16, 2007

Related U.S. Application Data
(60) Provisional application No. 60/499,317, filed on Aug. 29, 2003.

(51) Int. Cl. A61K 31/711; A61K 39/395; A61P 15/16; A61P 15/18

(52) U.S. Cl. 424/130.1; 435/375; 514/44

(57) ABSTRACT

The present invention is directed to methods and compositions for use in the diagnosis and treatment of reproductive fertility. More particularly, it is directed to methods and compositions that can be used in male and female contraception and fertility.

### FIELD OF THE INVENTION

The present invention generally relates to reproductive fertility and, more particularly, to methods and compositions that can be used in male and female contraception and fertility.

### BACKGROUND OF THE INVENTION

A novel protein of the structural maintenance of chromosomes (SMC) family of proteins, SMC1β, was recently discovered (Revenkova et al., Mol. Cell. Biol. 21: 6984-6998, 2001). SMC proteins are generally involved in chromosome dynamics, for example, in chromosome condensation during mitosis or in the alignment of the newly synthesized DNA molecules after DNA replication (“sister chromatid cohesion”) (reviewed in Jessberger, Nature Reviews, Mol. Cell. Biol. 3: 767-778, 2002). SMC proteins are evolutionary highly conserved and found in prokaryotes, as well as, in all eukaryotes examined, including humans. SMC proteins show a characteristic protein design with two globular domains at each terminus separated by an extended coiled-coil domain that at its center bears a third globular domain, the flexible hinge region. The terminal globular domains carry specific functional motifs such as the Walker A box, Walker B box (both involved in NTP binding and hydrolysis), a conserved FSKY motif, and the signature motif LSGG (see Jessberger, supra, 2002).

SMC1β was isolated from mammalian (bovine, mouse, rat) testis nuclear extracts and turned out to be the first, and so far only, mammalian meiosis-specific SMC protein. SMC1β does not exist in prokaryotes, yeast, or other lower eukaryotes including the nematode C. elegans. SMC1β is similar to the canonical SMC1, but contains specific differences such as a unique C-terminal peptide sequence. The overall identity to SMC1 is limited to 45-79%, depending on the individual domain. Thus, it provides sufficient uniqueness to allow development of specific reagents such as small compounds or antibodies. The specificity of SMC1β for meiotic cells was demonstrated in several tissues by several methods that include Northern blot analyses of RNA, Western blot analyses of protein extracts using antibodies specific for SMC1β, immunofluorescence studies on tissue sections and on chromosome spreads, and by immunoprecipitation from various extracts (Revenkova et al., supra, 2001; Eijpe et al., J. Cell. Biol., 160: 657-670, 2003). SMC1β was found to be exclusively expressed in meiotic cells, starting with the leptotene phase of meiosis I, and continues to be expressed until metaphase of meiosis II. In meiotic cells, SMC1β localizes to the core of meiotic chromosomes.

A role for SMC1β in sister chromatid cohesion of meiotic chromosomes throughout meiosis I and meiosis II has also been revealed. Evidence indicates that SMC1β, within a multiprotein complex, is acting as the “clamp” that holds sister chromatids together during meiosis (Revenkova et al., 2001). This phenomenon is absolutely required for meiotic progression and thus for spermatogenesis and oogenesis. Consequently, the absence of SMC1β is thought to have a deleterious effect on spermatogenesis and/or oogenesis. Methods and compositions for exploiting these finding are discussed in further detail in the present specification.

### SUMMARY OF THE INVENTION

The present invention relates to a structural maintenance of chromosome protein, SMC1β, and uses thereof. More specifically, the present invention provides methods for inducing or inhibiting fertility, comprising administering to an animal an effective therapeutic amount or an effective contraceptive amount, of an agent that either inhibits or induces SMC1β expression or activity.

In one aspect, therefore, the present invention provides a method for inducing infertility in an animal. Such a method generally would comprise inhibiting SMC1β expression or activity in said animal by contacting said animal with a nucleic acid selected from the group consisting of a nucleic acid that is an antisense SMC1β nucleic acid and a compound 8 to 80 nucleotides in length targeted to a nucleic acid molecule encoding SMC1β, wherein said compound specifically hybridizes with nucleic acid molecule of SEQ ID NO: 1 or 3 and inhibits the expression of SMC1β. In certain embodiments, the compound, targeted to a nucleic acid molecule encoding SMC1β, comprises a nucleic acid that is 12 to 50 nucleotides in length. In another embodiment, the compound comprises a nucleic acid that is 15 to 30 nucleotides in length. In a further embodiment, the compound comprises a nucleic acid that is 20 to 25 nucleotides in length. The compound maybe an antisense oligonucleotide, a DNA oligonucleotide, or an RNA oligonucleotide. In a further embodiment, at least a portion of said compound hybridizes with RNA to form an oligonucleotide-RNA duplex.

In another aspect, the present invention provides a method for inducing infertility in an animal. Such a method generally would comprise administering to the animal an effective contraceptive amount of an agent that inhibits SMC1β expression or activity. The term “inhibits SMC1β expression or activity” means decreases, lowers, downregulates, or otherwise decreases the endogenous effects of SMC1β. In certain embodiments, the method may further comprise restoring fertility to the animal by ceasing administration of the agent that inhibits SMC1β expression or activity. In another embodiment, the infertility may be caused by blocking gametogenesis, such as spermatogenesis in the male and/or oogenesis in the female. In a further embodiment, spermatogenesis and/or oogenesis, is blocked by inhibiting meiosis in developing germ cells. In a certain embodiment, the meiosis is inhibited at prophase of meiosis I or later. The present invention also contemplates an agent that inhibits SMC1β expression or activity that is selected from the group consisting of: a nucleic acid construct, a small molecule antagonist of SMC1β, apeptidomimetic antagonist of SMC1β, and an anti-SMC1β antibody. In another embodiment, the agent is administered in a composition comprising a pharmaceutically acceptable carrier. In a further embodiment, the pharmaceutically acceptable carrier is an adjuvant, solubilizer, stabilizer, diluent, anti-oxidant, liposome, micelle, or patch. In a preferred embodiment of the invention, the animal is human.

In another aspect, the present invention relates a method of treating infertility in an animal. Such a method generally would comprise administering to the animal an effective therapeutic amount of exogenous SMC1β or an agent that induces SMC1β expression or activity. The term “induces SMC1β expression or activity” means increases, augments, upregulates, or otherwise increases the endogenous effects of SMC1β. In certain embodiments, the infertility is treated by stimulating gametogenesis, such as spermatogenesis in the male or oogenesis in the female. In other embodiments, the spermatogenesis or oogenesis is stimulated by inducing meiosis in developing germ cells. In a certain embodiment, the meiosis is induced at or after prophase of meiosis I. The present invention also contemplates an agent that is selected from the group consisting of: a nucleic acid construct that encodes the SMC1β polypeptide, an SMC1β polypeptide, a small molecule agonist of SMC1β, and a peptidomimetic agonist of SMC1β. In another embodiment, the agent is administered in a composition comprising a pharmaceutically acceptable carrier. In a further embodiment, the pharmaceutically acceptable carrier is an adjuvant, solubilizer, stabilizer, diluent, anti-oxidant, liposome, micelle, or patch. In another embodiment, the agent is administered orally, parenterally, topically, transdermally, systemically, intravenously, intraarterially, intraperitoneally, or intramuscularly. In one embodiment, the administration is to the testis. In a certain embodiment, this administration to the testis is by a route selected from the group consisting of: injection, implantation, and transdermal application. In another embodiment, the administration is to the ovary. In a further embodiment, the administration to the ovary is by injection or implantation. In a preferred embodiment of the invention, the animal is human.

The present invention also contemplates a method of inhibiting meiosis in germ cells. Such a method generally would comprise inhibiting the expression or activity of SMC1β in the cells. In one embodiment, the germ cells are spermatocytes. In another embodiment, the germ cells are oocytes. In a further embodiment, meiosis is inhibited at prophase of meiosis I. In certain embodiments, the cells are treated either in vitro or in vivo. In another embodiment, cells are treated in an animal subject. Preferably, the subject is human. In a further embodiment, the method comprises contacting the cells with an agent that reduces the expression or activity of SMC1β. In one embodiment, the agent is a nucleic acid construct. In another embodiment, the agent is a small molecule antagonist of SMC1β. In a further embodiment of the invention, the agent is a peptidomimetic antagonist of SMC1β. In a different embodiment, the agent is an anti-SMC1β antibody. In a further aspect, the agent is administered in a composition comprising a pharmaceutically acceptable carrier. Preferably, the pharmaceutically acceptable carrier is an adjuvant, solubilizer, stabilizer, diluent, anti-oxidant, liposome, micelle, or patch.

The present invention further contemplates a method of inducing meiosis in germ cells. Such a method generally comprises inducing the expression or activity of SMC1β in the cells. In one embodiment, the germ cells are spermatocytes. In another embodiment, the germ cells are oocytes. In a certain embodiment, meiosis is induced at prophase of meiosis I or later. In further embodiments, the cells are treated either in vitro or in vivo. In another embodiment, cells are treated in an animal subject. Preferably, the subject is human. In a further embodiment, the method comprises contacting the cells with exogenous SMC1β or an agent that induces the expression or activity of SMC1β. In one embodiment, the agent is a nucleic acid construct that encodes the SMC1β polypeptide. In another embodiment, the agent is the SMC1β polypeptide. In a further embodiment of the invention, the agent is a mall molecule agonist of SMC1β. In a different embodiment, the agent is agent is a eptidomimetic agonist of SMC1β. In a further aspect, the agent is administered in a composition comprising a pharmaceutically acceptable carrier. Preferably, the pharmaceutically acceptable carrier is an adjuvant, solubilizer, stabilizer, diluent, anti-oxidant, liposome, micelle, or patch.

The invention also contemplates a method for treating a disorder in an animal resulting from decreased levels of SMC1β polypeptide. Such a method generally comprises administering to an animal the SMC1β polypeptide or the nucleic acid encoding the polypeptide of SMC1β to the animal.

In a different aspect, the invention contemplates a method of diagnosing a disorder or susceptibility to a disorder in an animal caused by or resulting from abnormal levels of SMC1β polypeptide. This method generally comprises: a) determining the presence or amount of expression or activity of the SMC1β polypeptide or a nucleic acid encoding the polypeptide of SMC1β in a sample; and b) comparing the level of SMC1β polypeptide or the nucleic acid encoding the polypeptide of SMC1β in a biological, tissue, or cellular sample from normal animals or the animal at an earlier time, wherein susceptibility to the disorder is based on the presence or amount of expression or activity of the SMC1β polypeptide or the nucleic acid encoding the polypeptide of SMC1β. In one embodiment, the disorder is selected from the group consisting of infertility, a pathological condition, and a nondisjunction syndrome.

The present invention also contemplates compositions comprising exogenous SMC1β or agents that induce SMC1β expression or activity and a pharmaceutically acceptable carrier. In one embodiment, the agent is selected from the group consisting of: a nucleic acid construct that encodes SMC1β polypeptide, SMC1β polypeptide, a small molecule agonist of SMC1β and a SMC1β peptidomimetic agonist. In another embodiment, the pharmaceutically acceptable carrier is an adjuvant, solubilizer, stabilizer, diluent, anti-oxidant, liposome, micelle, or patch.

The present invention also contemplates compositions comprising agents that reduce SMC1β expression or activity and a pharmaceutically acceptable carrier. In one embodiment, the agent is selected from the group consisting of: a nucleic acid construct that encodes SMC1β in an antisense orientation, a selective binding agent of SMC1β polypeptide, a small molecule antagonist of SMC1β, and a SMC1β peptidomimetic antagonist. In another embodiment, the pharmaceutically acceptable carrier is an adjuvant, solubilizer, stabilizer, diluent, anti-oxidant, liposome, micelle, or patch. In a further embodiment, the polynucleotide is contained with a vector.

In another aspect, the invention contemplates diagnostic reagents. Such diagnostic reagents generally comprise a detectably labeled polynucleotide encoding the SMC1β polypeptide, or a fragment, variant or homolog thereof. In one embodiment, the labeled polynucleotide is a first-strand cDNA.

A further aspect of the invention provides for methods for detecting the presence of SMC1β nucleic acids in a biological sample. Such a method comprises the steps of: a) providing a biological sample suspected of containing SMC1β nucleic acids; b) contacting the biological sample with a diagnostic reagent comprising a detectably labeled polynucleotide encoding the SMC1β polypeptide, or a fragment, variant or homolog thereof under conditions, wherein the diagnostic reagent will hybridize with SMC1β nucleic acids contained in the biological sample; c) detecting hybridization between SMC1β nucleic acid in the biological sample and the diagnostic reagent; and d) comparing the level of hybridization between the biological sample and diagnostic reagent with the level of hybridization between a known concentration of SMC1β nucleic acid and the diagnostic reagent, thereby detecting the presence of an SMC1β nucleic acid in the sample. In one embodiment, the polynucleotide molecule is DNA. In another embodiment, the polynucleotide molecule is RNA.

The present invention also provides methods for detecting the presence of SMC1β nucleic acids in a tissue or cellular sample. Such methods generally comprise the steps of: a) providing a tissue or cellular sample suspected of containing SMC1β nucleic acids; b) contacting the tissue or cellular sample with a diagnostic reagent comprising a detectably labeled polynucleotide encoding the SMC1β polypeptide, or a fragment, variant or homolog thereof under conditions, wherein the diagnostic reagent will hybridize with SMC1β nucleic acids; c) detecting hybridization between SMC1β nucleic acid in the tissue or cellular sample and the diagnostic reagent; and d) comparing the level of hybridization between the tissue or cellular sample and diagnostic reagent with the level of hybridization between a known concentration of SMC1β nucleic acid and the diagnostic reagent. In one embodiment, the polynucleotide molecule is DNA. In another embodiment, the polynucleotide molecule is RNA.

In another aspect, the invention provides methods for screening agents that modulate meiosis in germ cells. Such methods generally comprise: a) providing a cell expressing SMC1β; b) contacting the cell with a candidate modulating agent; c) monitoring the cell for a change in meiotic activity in the presence and absence of modulating agent; and d) identifying a candidate modulating agent as a modulating agent when the meiotic activity differs in the presence or absence of the agent. In one embodiment, the modulating agent increases meiosis. In another embodiment, the modulating agent decreases meiosis. In other embodiments, the cell may be a spermatocyte or an oocyte. In a further embodiment, the cell is from a transgenic, non-human animal. In certain embodiments, the contacting of the cells with modulating agents may be carried out either in vitro or in vivo. In other embodiments, the candidate modulating agent may be selected from the group consisting of: a nucleic acid construct that reduces the expression or activity of SMC1β, a nucleic acid construct that increases the expression or activity of SMC1β, an antibody of SMC1β, a small molecule antagonist of SMC1β, and a peptidomimetic antagonist of SMC1β.

In yet another aspect, the invention provides for compositions comprising candidate modulating agent of meiosis identified by the methods described herein and a pharmaceutically acceptable carrier. In one embodiment, the pharmaceutically acceptable carrier is an adjuvant, solubilizer, stabilizer, diluent, anti-oxidant, liposome, micelle, or patch.

A further aspect of the invention provides a method of modulating levels of SMC1β in an animal. Such a method generally comprises administering to the animal the composition comprising a candidate modulating agent of meiosis identified by the methods described herein and a pharmaceutically acceptable carrier.

Another aspect provides a method of modulating meiosis in an animal. Such a method generally comprises administering to the animal the composition comprising a candidate modulating agent of meiosis identified by the methods described herein and a pharmaceutically acceptable carrier.

The invention also contemplates methods for identifying agents that modulate SMC1β expression or activity in germ cells. Such methods generally comprise the following steps: a) providing a cell expressing SMC1β; b) contacting the cell with a candidate modulating agent; c) monitoring the cell for a change in SMC1β expression or activity in the presence and absence of modulating agent; and d) identifying a candidate modulating agent as a modulating agent when SMC1β expression or activity differs in the presence or absence of the agent.

The invention further contemplates methods for screening agents that inhibit SMC1β expression or activity in germ cells. Such methods generally comprise the following steps: a) introducing an inducible expression construct of SMC1β into a somatic cell; b) contacting said cell with a candidate inhibitor of SMC1β; and c) monitoring said cell for an increase in proliferation; wherein an increase in cell proliferation indicates that the agent is a SMC1β inhibiting agent.

In another aspect, the invention contemplates a composition comprising a candidate modulating agent of meiosis or SMC1β expression or activity identified according to the method for screening agents described herein and a pharmaceutically acceptable carrier. In one embodiment, the pharmaceutically acceptable carrier is an adjuvant, solubilizer, stabilizer, diluent, anti-oxidant, liposome, micelle, or patch. Another embodiment of the invention provides methods of modulating levels of SMC1β in an animal comprising administering to the animal the composition comprising the modulating agent(s). In a further embodiment, the invention provides methods of modulating meiosis in an animal comprising administering to the animal the composition comprising the modulating agent(s).

The present invention also provides transgenic non-human animals whose genomes comprise ahomozygous null mutation in the endogenous SMC1β gene, wherein the non-human animal exhibits abnormal development of the germ cells. In one embodiment, the transgenic non-human animal is a mouse.

The invention further provides isolated cells, or a purified preparation of cells, from a transgenic non-human animal whose genome comprises a homozygous null mutation in the endogenous SMC1β gene, wherein production of functional SMC1β is inhibited.

In a different aspect, the invention provides methods of evaluating a fertility treatment. Such methods generally comprise: a) administering the treatment to a transgenic mouse whose genome comprises a homozygous null mutation in the endogenous SMC1β gene, wherein the mouse exhibits abnormal development of the germ cells and is infertile; and b) determining the effect of the treatment on fertility of the mouse, thereby evaluating the fertility treatment. In one embodiment, the treatment is evaluated in vivo, while in a different embodiment the treatment is evaluated in vitro. Furthermore, in other embodiments, the treatment may be determined by sperm count, testicular size, oocyte morphology, sperm morphology, gamete morphology, chromosome morphology, the ability of chromosomes to pair, the ability of the mice to mate and produce offspring, the ability of the mice to have normal estrous cycles, and ovarian morphology.

The invention also contemplate an expression construct comprising a nucleic acid encoding a SMC1β polypeptide, fragment, or variant thereof and a heterologous germ cell specific promoter operably linked to the construct. In a certain embodiment, the nucleic acid is in a sense orientation with respect to the promoter. In a different embodiment, the nucleic acid is in an antisense orientation with respect to the promoter. In one embodiment, the construct is contained within a viral vector. In a further embodiment, the promoter is a testis specific promoter. Still further, the promoter is the promoter for phosphoglycerate kinase 2. In a different embodiment, the promoter is an oocyte specific promoter.

The invention also contemplates recombinant host cells, wherein the cells are transformed with the any of the constructs described herein. Still further, the invention contemplates compositions comprising any of the constructs of described herein and a pharmaceutically acceptable carrier. In one embodiment, the pharmaceutically acceptable carrier is an adjuvant, solubilizer, stabilizer, diluent, anti-oxidant, liposome, micelle, or patch.

In another aspect, the invention contemplates a device, comprising: a) a membrane suitable for implantation; and b) the composition comprising any of the constructs described herein encapsulated within the membrane, wherein the membrane is permeable to the composition.

In yet another aspect, the invention provides a device, comprising: a) a membrane suitable for implantation; and b) the recombinant host cells transformed with any of the constructs described herein encapsulated within the membrane, wherein the cells secrete polypeptide, and wherein the membrane is impermeable to materials detrimental to the cells.

In another aspect, the invention provides a selective binding agent that is an antibody or a fragment thereof of SMC1β polypeptide. In one embodiment, the antibody or fragment thereof specifically binds SMC1β polypeptide. In a still further embodiment, the antibody is a monoclonal antibody.

In a further aspect, the invention contemplates the use of the compounds claimed herein in the manufacture of a medicament substantially as herein described and illustrated.

The invention also contemplates compounds for inducing the expression of a heterologous gene in a germ cell comprising an SMC1β promoter comprising a sequence of SEQ ID NO: 12 or 13 operably linked to said heterologous gene.

In another aspect, the invention contemplates methods of inducing expression of a heterologous gene in a germ cell comprising contacting said germ cell with an expression construct comprising said heterologous gene operably linked to an SMC1β promoter comprising a sequence of SEQ ID NO: 12 or 13, under conditions effective to allow expression of said heterologous gene.

### BRIEF DESCRIPTION OF THE DRAWING

FIG. 1 shows the alignment of mouse and human SMC1β promoter nucleotide sequences (SEQ ID NOS: 12 and 13, respectively).

### DETAILED DESCRIPTION OF THE PREFERRED EMBODIMENTS

Germ-cell specific proteins are target candidates in the regulation and/or manipulation of fertility and infertility. SMC1β, a germ cell-specific protein, was isolated from mammalian testis nuclear extracts and identified to be a mammalian meiosis-specific SMC protein (Revenkova et al., supra, 2001). SMC1β was found to be exclusively expressed in meiotic cells, starting with the leptotene phase of meiosis I, and continues to be expressed until metaphase of meiosis II. In meiotic cells, SMC1β localizes to the core of meiotic chromosomes.

A role for SMC1β in sister chromatid cohesion of meiotic chromosomes throughout meiosis I and meiosis II has also been revealed. Evidence indicates that SMC1β, within a multiprotein complex, is acting as the “clamp” that holds sister chromatids together during meiosis (Revenkova et al., supra, 2001). This phenomenon is absolutely required for meiotic progression and thus for spermatogenesis and oogenesis. Consequently, the absence of SMC1β has been shown to have a deleterious effect on spermatogenesis and is thought to have a deleterious effect on oogenesis.

The present invention addresses a role for SMC1β in the regulation of fertility and infertility. More specifically, the present invention contemplates methods for inducing fertility (or effecting contraception) and treating infertility. The inventors of the present application have shown that mutations in SMC1β resulted in infertility. More specifically, the inventors discovered that mutations in SMC1β inhibited meiosis in germ cells, especially at prophase I or later. In addition, it was determined that both female and male mice were infertile as a result of this mutation. These observations showed that normal SMC1β expression or activity is an important step in the process of gametogenesis. Thus, these findings provide the first evidence of a role for SMC1β in the regulation of gametogenesis and fertility.

The present invention further contemplates the use of an inhibitor of SMC1β for the treatment of infertility disorders. The use of SMC1β compositions also are contemplated for treatment of biological materials outside the body to determine a diagnostic result. The present invention further contemplates the use of an inhibitor of SMC1β for the manufacture of a medicament for the treatment of infertility disorders.

Discussed in further detail herein below are the mechanisms by which mutations in SMC1β lead to the inhibition of meiosis and infertility. Furthermore, the present findings provide insights into the role of SMC1β in normal fertility. Also described are methods and compositions for making and using various animal models of SMC1β. The transgenic mouse knockout and knockin models will likely serve as useful models for the exploration of potential mechanisms of SMC1β's role in gametogenesis. Such models may also be used in assays to identify factors that cooperate with loss of wild-type SMC1β in the regulation of meiosis and gametogenesis and to develop agents for the therapeutic intervention of fertility and infertility.

Definitions

The term “infertility” refers to the state of not being fertile or not being able to conceive offspring. It may be a temporary state, such as that which is induced by a method of contraception or it may be a permanent state, which may be of known or unknown etiology. Infertility may occur in either the male or the female or both.

The term “agent that inhibits SMC1β expression or activity” refers to any molecule or molecules (e.g., nucleic acid, binding agent, antibody, peptibody, etc.) which can act directly or indirectly to downregulate expression or activity of the SMC1β polynucleotide or polypeptide and block the subsequent expression or activity of SMC1β.

The term “agent that induces SMC1β expression or activity” refers to any molecule or molecules (e.g., nucleic acid, polypeptide, etc.) which can act directly or indirectly to upregulate expression or activity of the SMC1β polynucleotide or polypeptide and induce the subsequent expression or activity of SMC1β.

The term “nucleic acid construct” refers to any nucleic acid molecule or molecules (e.g. either alone or in conjunction with a vector, promoter, enhancer, terminator, etc.). This term includes, but is not limited to, DNA, RNA, oligonucleotides, including upstream and downstream regulators of nucleic acid expression.

The terms “effective amount” and “therapeutically effective amount” each refer to the amount of an SMC1β polypeptide, an SMC1β nucleic acid molecule, or an agent that induces SMC1β expression or SMC1β activity used to support an observable level of one or more biological activities of the SMC1β polypeptide set forth herein.

The term “expression vector” refers to a vector which is suitable for use in a host cell and contains nucleic acid sequences which direct and/or control the expression of heterologous nucleic acid sequences. Expression includes, but is not limited to, processes such as transcription, translation, and RNA splicing, if introns are present.

The term “host cell” is used to refer to a cell which has been transformed, or is capable of being transformed with a nucleic acid sequence and then of expressing a selected gene of interest. The term includes the progeny of the parent cell, whether or not the progeny is identical in morphology or in genetic make-up to the original parent, so long as the selected gene is present.

The terms “pharmaceutically acceptable carrier” or “physiologically acceptable carrier” as used herein refer to one or more formulation materials suitable for accomplishing or enhancing the delivery of the SMC1β polypeptide, SMC1β nucleic acids molecule, or SMC1β selective binding agent as a pharmaceutical composition.

The term “selective binding agent” refers to a molecule or molecules having specificity for a SMC1β polypeptide. As used herein the terms “specific” and “specifically” refer to the ability of the selective binding agents to bind to SMC1β polypeptides and not to bind to non-SMC1β polypeptides. It will be appreciated, however, that the selective binding agents may also bind orthologs of the SMC1β polypeptide.

The term “transduction” is used to refer to the transfer of genes from one bacterium to another, usually by a phage. “Transduction” also refers to the acquisition and transfer of eukaryotic cellular sequences by retroviruses.

The term “transfection” is used to refer to the uptake of foreign or exogenous DNA by a cell, and a cell has been “transfected” when the exogenous DNA has been introduced inside the cell membrane. A number of transfection techniques are well known in the art and are disclosed herein. See, for example, Graham et al., Virology, 52: 456, 1973; Sambrook et al., Molecular Cloning, A Laboratory Manual, Cold Spring Harbor Laboratories (New York, 1989); Davis et al., Basic Methods in Molecular Biology, Elsevier, 1986; and Chu et al., Gene, 13: 197, 1981. Such techniques can be used to introduce one or more exogenous DNA moieties into suitable host cells.

The term “transformation” as used herein refers to a change in a cell's genetic characteristics, and a cell has been transformed when it has been modified to contain new DNA. For example, a cell is transformed where it is genetically modified from its native state. Following transfection or transduction, the transforming DNA may recombine with that of the cell by physically integrating into a chromosome of the cell, it may be maintained transiently as an episomal element without being replicated, or it may replicate independently as a plasmid. A cell is considered to have been stably transformed when the DNA is replicated with the division of the cell.

The term “vector” is used to refer to any molecule (e.g., nucleic acid, plasmid, or virus) used to transfer coding information to a host cell.

The section headings used herein are for organizational purposes only and are not to be construed as limiting the subject matter described therein. All references cited in this application are expressly incorporated by reference herein.

A. Involvement of SMC in Chromosome Dynamics

The present section provides a description of the involvement of structural maintenance of chromosome (SMC) proteins in DNA and chromatin dynamics to the extent that such a description will facilitate a better understanding of the methods and compositions of the present invention.

SMC1β belongs to the family of SMC proteins, whose members act in DNA and chromatin dynamics. In particular, they are important for chromosome condensation and sister chromatid cohesion, and involved in DNA repair and recombination. All these processes are at the heart of meiosis, but have been specifically modified to serve the purpose of generating germ cells. Strong evidence suggests that SMC1β, which exists only in higher eukaryotes, is central to meiotic sister chromatid cohesion and DNA recombination. Its function is highly likely to be strictly controlled in a meiosis stage-specific and chromosomal location-specific manner. The expression of SMC1β may be regulated by specific transcription factors and proteolysis/phosphorylation events. Thus, SMC1β is a potential target for interfering with male meiosis.

Meiosis features unique and important chromosome processes. These include the formation of meiosis-specific protein-DNA structures like the synaptonemal complex (SC), DNA recombination, and reductional segregation of chromosomes. There is accumulating evidence for a requirement for SMC proteins in these processes.

The evolutionary highly conserved eukaryotic SMC protein family includes six members named SMC1 to SMC6 and is involved in several key dynamic DNA and chromatin processes (reviews: Jessberger et al., Curr. Opin. Genet. Dev. 8:254-259, 1998; Strunnikov, Trends Cell Biol. 8:454-459, 1998; Hirano, Curr. Opin. Cell. Biol. 10:317-322, 1998, Hirano, Genes Dev. 13:11-19, 1999, Hirano, Genes Dev. 16:399-414,2002; Strunnikov and Jessberger, Europ. J. Biochem. 263:6-13, 1999; Cobbe and Heck, J. Struct. Biol. 129:123-143, 2000; and Jessberger, Nat. Rev. Mol. Cell. Biol. 3:767-778, 2002). The SMC family was first defined as such in 1994 and has since then attracted increasing attention. The first publication on mammalian SMC proteins was by Jessberger et al. (1996a). To date, the best documented processes in which SMC proteins are involved are chromosome condensation and sister chromatid cohesion. A role in DNA recombination and the response to DNA damage has also been demonstrated. A fourth role of SMC proteins (SMC2, SMC4) is in gene dosage compensation in nematodes (Chuang et al., Science 274:1736-1738, 1996; Lieb et al., Cell 92:1-20, 1998). The six subfamilies of SMC proteins can be grouped according to the three types of heterodimers that they form: SMC1/SMC3, SMC2/SMC4, and SMC5/SMC6. The SMC5 and SMC6 proteins (Spr18 and Rad18 proteins in S. pombe) belong to a more ancestral family and act in DNA repair (Lehmann et al., Mol. Cell. Biol. 15:7067-7080, 1995; Jones and Sgouros, Genome Biol. 2:RESEARCH0009, 2001). All the heterodimers constitute core components of larger multiprotein complexes that carry out specific functions. One of the major SMC complexes is condensin, which contains the SMC2/4 heterodimer and several non-SMC subunits, and is necessary for mitotic chromosome condensation (Strunnikov et al., Genes and Dev. 9:587-599, 1995; Sutani and Yanagida, Nature, 388:798-801, 1997; Hirano et al., Cell 89:511-521, 1997).

The other heterodimer, SMC1/3 is a component of at least three protein complexes. Genetic studies in S. cerevisiae and biochemical analysis in X. laevis egg extracts revealed a requirement for the SMC1/3 proteins in mitotic sister chromatid cohesion (Guacci et al., Cell 91:47-58, 1997; Michaelis et al., Cell 91:35-46, 1997; Losada et al., Genes Devel. 12:1986-1997, 1998). The mitotic cohesin complex contains, besides SMC1/3, at least two other polypeptides, the Rad21 (Scc1p/Mcd1p), and the Scc3 proteins. The SMC1/3 heterodimer has also been identified as constituent of the recombination complex, RC-1, which was isolated from bovine thymus and is present in a variety of somatic cells (Jessberger et al., Journ. Biol. Chem. 268:15070-15079, 1993; Jessberger et al., EMBO-J. 15:4061-4068, 1996; Stursberg et al., Gene 228:1-12, 1999). RC-1 catalyzes SMC protein-dependent cell-free recombinational repair of gaps and deletions. The presence of the SMC1/3 heterodimer in these multiprotein complexes furthered speculations about an SMC-mediated functional link of sister chromatid cohesion and recombinational repair (Jessberger et al., Curr. Opin. Genet. Dev. 8:254-259, 1998; Hirano, Curr. Opin. Cell. Biol. 10:317-322, 1998; Strunnikov and Jessberger, Europ. J. Biochem. 263:6-13, 1999). This idea was supported by the recent report on DNA damage-dependent phosphorylation of SMC1 by ATM kinase (Kim et al., Genes & Dev. 16:560-570, 2002; Yazdi et al., Genes Dev. 16:571-582, 2002).

Features of SMC proteins are determined by specific properties of their five domains. The N-terminal domains invariably contain an NTP binding motif, the Walker A box. The C-termini bear a DA box with a Walker B-like sequence and the LSGG signature motif, typical for the ABC ATPase family of proteins. The hinge domain is characterized by a set of four highly conserved glycine residues often found in flexible regions in a protein, with the consensus sequence G(X)₆G(X)₃GG. While the N-terminal domain does not bind DNA, the C-terminal domain, hinge domain, and a coiled-coil domain do (Akhmedov et al., J. Biol. Chem. 273:24088-24094, 1998; Akhmedov et al., J. Biol. Chem. 274:38216-38224, 1999; Hirano et al., EMBO J. 21:5733-5744, 2002). Most domains show a strong preference for double-stranded (ds) DNA substrates, and a high specificity for dsDNA molecules, which are able to adopt secondary structures. Good binding substrates were also palindromic and/or A/T-rich sequences such as scaffold associated regions and centromere DNA-derived fragments (Akhmedov et al., J. Biol. Chem. 273:24088-24094, 1998; Akhmedov et al., J. Biol. Chem. 274:38216-38224, 1999). Chromatin immunoprecipitation experiments confirmed the preferential localization of Smc3p or other cohesin components to A/T-rich and centromeric regions in yeast (Blat and Kleckner, Cell 98:249-259, 1999; Megee et al., Mol. Cell 1999; Tanaka et al., Cell, 98:847-858, 1999; and Laloraya et al., Cell Biol. 151:1047-1056, 2000).

The hinge domain is thought to provide flexibility to the SMC molecules by allowing opening or closing of the two-armed structure. By electron microscopy, Melby et al. J. Cell. Biol. 142:1595-1604, (1998) demonstrated, for prokaryotic homodimeric SMC proteins, that the two arms of SMC molecules could move around the central hinge. These dimers are antiparallel, bearing an N- and C-terminus at each end. A series of pull-down assays using combinations of wildtype or mutant yeast SMC1 and SMC3 proteins expressed in insect cells, and crystal data from a bacterial homodimer, yielded data that support a model calling for a heterodimer made of SMC proteins folded back onto themselves, linked at their hinge domains, and potentially embracing DNA between its two arms.

In meiotic cells, meiosis-specific SMC complexes have evolved. Two of the non-SMC subunits of cohesin are replaced by meiosis-specific proteins: Rad21 is replaced by Rec8, and Scc3 by STAG3 (reviewed by Petronczki et al., Cell 112:423-440, 2003). In 2001, the identification of a novel, meiosis-specific SMC protein was reported (Revenkova et al., Mol. Cell. Biol. 21:6984-6998, 2001).

B. The Discovery of SMC1β

The present section provides an overview of the discovery of SMC1β to the extent that such an overview will facilitate a better understanding of the methods and compositions of the present invention.

Immunoprecipitation using anti SMC3 antibodies and testis nuclear extracts revealed an additional SMC protein, SMC1β, identified as a meiosis-specific isoform of SMC1, hence renamed SMC1α. SMC1β displayed a chromosomal localization pattern much like SMC3, i.e. in spermatocytes it was found to be associated with the entire chromosome from prophase I until metaphase I. At that stage, it disappears from the chromosome arms, but it is retained at the centromeres, which are the sites of continued sister chromatid cohesion until metaphase/anaphase of meiosis II. At anaphase II, SMC3 and SMC1β, along with Rec8, which behaves similar but not identically, dissociate also from the centromeres allowing the sister chromatids to be separated. SMC1α, however, is more loosely associated with the chromosomes, and disappears entirely during meiosis I (Eijpe et al., J. Cell Sci. 113:673-682, 2000; Revenkova et al., Mol. Cell. Biol. 21:6984-6998, 2001). Therefore, SMC1β, but not SMC1α, is now thought to be responsible for centromeric sister chromatid cohesion in meiosis. SMC1β was also found at chiasmata and on bridges between homologous chromosomes in meiosis I and may thus be involved in meiotic recombination as well. Smc3p in S. cerevisiae has indeed been shown to be required for meiotic recombination and meiotic sister chromatid cohesion (Klein. et al., Cell 98: 91-103, 1999). However, there is no protein orthologue to SMC1β in lower eukaryotes like yeast or even C. elegans. SMC1β is highly conserved between mouse and man (82% amino acid identity). These findings suggest that there are several different SMC-containing cohesin-like complexes with specialized functions in meiotic cells.

The compositions of the SMC1β-based complex are not yet precisely known. There may be two SMC1β complexes, one specific for the centromere, the other for the chromosome arms. It is also unknown how association and dissociation of SMC1β from either the chromosome arms in meiosis I or the centromeres in meiosis II is regulated. Furthermore, it remains to be elucidated, how meiosis-specific expression of SMC1β is achieved and timed.

The unexpected finding of a meiosis-specific SMC protein, and the initial characterization of SMC1β was described in Revenkova et al., Mol. Cell. Biol. 21:6984-6998, 2001. In that study it was determined that SMC1β is an isoform of SMC1α with amino acid identity in conserved domains of 45-79%; SMC1β contains an unusual, highly basic 28 amino acid peptide at its C-terminus; SMC1β forms a dimer with SMC3; SMC1β protein is exclusively found in meiotic cells; SMC1β, together with SMC3, localizes along the axial elements of the synaptonemal complex; and SMC1β dissociates from meiotic chromosomes in two steps: from their arms at metaphase I, from their centromeres at metaphase II. It is highly likely that SMC1β (and not SMC1α) is very likely responsible for sister chromatid cohesion up to anaphase II. The expression of SMC1β must be a strictly regulated process, and its function in association with meiotic chromosomes needs to be tightly controlled.

C. The SMC1β Promoter

The present section provides a characterization of the SMC1β promoter, which provides some insight into the regulation of the SMC1β gene. The characterization of the SMC1β promoter allows for better understanding and further development of the methods and compositions of the present invention. Such a promoter sequence may be used to drive the expression of SMC1β-derived nucleic acid sequences in a variety of recombinant techniques described herein, or alternately may be used to effect germ cell-specific expression of non-SMC1β nucleic acid sequences.

Northern blotting of RNA from a variety of mouse tissues was performed using a 616-bp 5′ fragment of SMC1β cDNA as a probe. This experiment confirmed testis-specific expression of the gene. The specific signal of about 4.5 kb was not seen in RNA from any other tissue. The same probe was also used to analyze RNA prepared from purified spermatocytes, and the same 4.5-kb mRNA was detected.

The mouse gene for SMC1β is located on chromosome 15 (Ensemble gene ID ENSMUSG00000022432; mouse SMC1β mRNA, GenBank accession number NM_{—}080470 (SEQ ID NO: 1); mouse SMC1β protein, GenBank accession number NP_{—}536718 (SEQ ID NO: 2)]). The human homolog was found in a syntenic region on human chromosome 22 (human SMC1β; GenBank accession number NT_{—}011522), and is known as human SMC1β, GeneID 27127 [human SMC1β mRNA, GenBank accession number NM_{—}148674 (SEQ ID NO: 3); human SMC1β protein, GenBank accession number NP_{—}683515 (SEQ ID NO: 4)]. The coding sequence position in chromosome 22, contig NC_{—}000022, is from 44060974 to 44129985. In both genomes, the locus contains another gene located in close proximity to the SMC1β gene. In the human genome, the nucleotide sequence for the second gene which lies in close proximity, GeneID 26150, is GenBank accession number NM_{—}015653 (SEQ ID NO: 5) which encodes the amino acid sequence, GenBank accession number NP_{—}056468 (SEQ ID NO: 6). In the mouse genome, the nucleotide sequence for the second gene which lies in close proximity is GenBank accession number AK016311 (SEQ ID NO: 7), which encodes the protein, GenBank accession number BAB30190 (SEQ ID NO: 8; also identified as GenBank accession number AAH09904). The two genes are arranged in a head-to-head configuration. In the mouse genome, the distance between the 5′ end of the SMC1β cDNA and the 5′ end of the AK016311 cDNA is only 142 bp. In the human genome, the start of a cDNA clone and SMC1β start codon are 162 bp apart. Therefore, it is very likely that the 5′ region of the SMC1β gene contains a bidirectional promoter, or two overlapping promoters.

The analysis of the human genome (Adachi and Lieber, Cell 109:807-809, 2002) revealed that a significant fraction of genes is organized in a divergent fashion with transcription start sites less than 1 Kb apart. For example, 319 genes located on chromosome 22 were analyzed, and 56 (18%) fell into this category. The fraction was even higher among the genes implicated in DNA repair (42% among 120 genes analyzed). The physiologic role of such organization for most of the cases still has to be elucidated. At the SMC1β locus the second gene encodes a 37-kDa protein product with unknown function. In human, transcripts of this gene were found in tumors, including germ cell tumors.

A striking characteristic feature of human bidirectional promoter regions is the presence of a CpG island between the genes (Adachi and Lieber, Cell 109:807-809, 2002). In the SMC1β locus the CpG island occupies the region from position 330 bp upstream to 140 bp downstream of the SMC1β start codon.

For analysis of the putative regulatory region upstream of the SMC1β gene, and in order to attempt to create a SMC1β deficient mouse, a BAC clone was isolated from a mouse genomic library. This BAC clone contains at least 6 kbp of sequence upstream of the SMC1β start site. A 437 bp fragment derived from this BAC clone, which terminates 70 bp upstream of the SMC1β start codon, was used as a probe for a Northern blot of total RNA from adult mouse tissues to analyze transcription of the AK016311 gene. A transcript of approximately 1.6 kbp was detected in RNA from the testis and purified spermatocytes, but not from kidney.

The sequence from position 2100 upstream to position 200 downstream of the SMC1β start codon was then analyzed by the PromoterInspector program, which predicts RNA polymerase II promoter regions in mammalian genomic sequences (Scherf et al, J. Mol. Biol. 297:599-606, 2000, Genomatix, Munich, GERMANY). The program identified a 220 bp fragment containing the SMC1β start codon as a promoter region. The program MatInspector (Genomatix, Munchen, GERMANY) was then used to identify potential transcription factor binding sites within this region. The same analysis was performed for the corresponding human sequence. The sites conserved between the mouse and the human regions include potential binding sites for members of the transcription factor families RFX, E2F, and the retinoic acid receptor (RAR) family.

By the alignment of 5′ regions of mouse and human SMC1β genes, the human SMC1β promoter region was identified as a potential promoter by the program PromoterInspector. Putative binding sites for 3 families of transcription factors were identified: RFX—Regulatory factor X family, E2F—E2F family, and RAR—retinoic acid receptor family. The starting positions of SMC1β cDNA and AK01631 cDNA were determined.

The RFX family of transcription factors contains 5 members in both the human and the mouse. RFX1 is expressed ubiquitously and forms homodimers and heterodimers with RFX2 and RFX3. Highest expression level of these proteins was found in testes (Reith et al, Mol Cell Biol 14:1230-1244, 1994) but so far no testis-specific target genes have been identified. RFX4 is closely related to RFX1-3 and detected only in testes (Morotomi-Yano et al, J Biol Chem 277:836-842, 2002). RFX4, transiently expressed in COS7 cells, interacts with RFX2 and RFX3. Target genes controlled by RFX4 are unknown. In S. pombe, the RFX homolog, sak1, regulates exit from the mitotic cycle (Wu and McLeod, Moll Cell Biol 15:1479-1488, 1995).

The E2F family of transcription factors includes at least six members, which control genes involved in DNA replication and repair, cell proliferation, differentiation and apoptosis (reviewed in Stevaux and Dyson, Curr. Opin. Cell Biol. 14:684-691, 2002). Knockout mice lacking E2F1 displayed testicular atrophy and developed a variety of tumors, predominantly reproductive tract sarcomas (Yamasaki et al., Cell 85:537-548, 1996). The overexpression of E2F1 in transgenic mice also caused testicular atrophy and sterility as a result of increased apoptosis in the germinal epithelium (Holmberg et al., Oncogene 17:143-155, 1998).

Retinoic acid receptors control numerous genes involved in cell proliferation and differentiation (reviewed in Wei, Annu. Rev. Pharmacol. Toxicol 43:47-72, 2003). There are two classes of nuclear receptors binding retinoids, RAR and RXR. RARα plays a critical role in spermatogenesis as demonstrated by mouse knockout studies. Disruption of the RARα gene causes degeneration of meiotic and post-meiotic testicular cells, similar to that observed in vitamin A-deficient animals (Lufkin et al., Proc Natl Acad Sci USA 90:7225-7229, 1993).

D. Making and Using Transgenic Animals of the Invention

Particular aspects of the present invention involve the production of transgenic animals. In particular, the first set of transgenic mice contemplated by the present invention are those which have a loss of the SMC1β phenotype. The second set of transgenic mice are those that conditionally express SMC1β. The rationale and methods and compositions for the production of these transgenic animals are provided in further detail herein below.

Those of skill in the art are aware of general techniques for making transgenic animals. Such techniques involve the integration of a given nucleic acid construct into the genome in a manner that permits the expression of a transgene or the knockout of an existing gene. Methods for producing transgenic animals are generally described by Wagner and Hoppe (U.S. Pat. No. 4,873,191, which is incorporated herein by reference), Palmiter and Brinster (Cell 41:343-345, 1985, which is incorporated herein by reference in its entirety), and in “Manipulating the Mouse Embryo; A Laboratory Manual” 2^{nd} edition (eds. Hogan, Beddington, Costantimi and Long, Cold Spripig Harbor Laboratory Press, 1994, which is incorporated herein by reference in its entirety). In the present application, the genes of interest are SMC1β and SMC1β-related genes. The wild-type SMC1β sequence is known to those of skill in the art [see e.g., Genebank accession no. AF303827; also known as GenBank accession no. NM_{—}080470 (SEQ ID NO: 1)], as may be used as the underlying sequence for the production of the transgenic mice.

Typical techniques for producing transgenic animals involve the transfer of genomic sequences by microinjection into a fertilized egg. The microinjected eggs are implanted into a host female, and the progeny are screened for the expression of the transgene. Transgenic animals may be produced from the fertilized eggs from a number of animals including, but not limited to, reptiles, amphibians, birds, mammals, and fish. Methods for the production and purification of DNA for microinjection are described in Hogan et al., Manipulating the Mouse Embryo (Cold Spring Harbor Laboratory, Cold Spring Harbor, N.Y., 1986), in Palmiter et al. Nature 300:611 (1982); the Qiagenologist, Application Protocols, 3^{rd} edition, published by Qiagen, Inc., Chatsworth, Calif.; and in Sambrook et al. Molecular Cloning: A Laboratory Manual (Cold Spring Harbor Laboratory, Cold Spring Harbor, N.Y., 1989).

The first set of transgenic animals of the present invention provide a model for determining whether the loss of SMC1β is sufficient for inducing infertility. Because the SMC1β and AK016311 genes are located very close to each other in a divergent orientation, it was assumed that it would be hardly possible to delete the first exon of SMC1β without affecting the expression of the AK016311 gene. The phenotype of the resulting mice would be very difficult to interpret. Therefore, the best approach was determined to be the deleting one of the central exons of SMC1β, which is located far downstream from the AK016311 gene. The amino acid sequence encoded by exon 10 of SMC1β comprises a large part (40%) of its hinge domain. The hinge domain has been shown to be important for the formation of the SMC heterodimers, and there is direct evidence for dimer formation by the SMC1β and SMC3 hinge domains. The SMC1β-SMC3 hinge domain, like other hinge domain dimers, is also required for DNA binding in vitro (Hirano and Hirano, EMBO J. 21:5733-5744, 2002; and Revenkova et al., Mol. Cell. Biol., 2001, in preparation) and may play a role in SMC-DNA interactions. Therefore, it was assumed that removing a large part of the hinge region would result in a non-functional protein. In addition, stop codons are present in the selective marker gene (neo) introduced in place of exon 10. Even in case the selective marker replacing exon 10 was transcriptionally skipped in the transgenic mouse, the resulting SMC1β protein, with a deletion of most of the hinge region, would be nonfunctional.

To generate a knockout construct, a probe derived from SMC1β cDNA was used to screen a mouse (strain 129SvJ) genomic library. The screen yielded one BAC clone that carried at least 21 kb of genomic DNA, which spanned about 6 kb of 5′ upstream region and at least 11 exons of SMC1β. The identity of the sequence was confirmed by partial sequencing and restriction endonuclease digestion. Several genomic DNA fragments were subcloned, and a 9.6 kb BamHI fragment, which spanned exons 8 to 11, was used for the generation of the targeting construct. A 7.8 kb subfragment of genomic DNA was subcloned into pBluescriptSK 3′ of a thymidinedkinase gene, which served for selection against random integrants. In the plasmid, the neomycin resistance marker gene replaced a 1.2-kb fragment containing SMC1β exon 10. The neomycin resistance gene was inserted in direct orientation in respect to SMC1β to avoid the production of antisense transcripts.

The construct was electroporated into W4/129S6 embryonic stem (ES) cells (Taconic Inc.). Production and analysis of targeted ES clones was performed as described (Matise et al, Production of targeted embryonic stem cell clones, In: Gene targeting: a practical approach, Chapter 3, Joyner A. L. (e.d.), Oxford University Press, New York, 2000). Four independent ES clones were isolated in which neomycin resistance marker replaced SMC1β exon 10. The frequency of targeted transformants was 1% (4/395 screened) homologous recombinants per total transformants. The structure of the targeted locus was verified by Southern hybridization of ES genomic DNA digested with BamHI with two external probes upstream and downstream of the targeted site. Blastocyst injection of the ES cells from four clones, followed by implantation into foster mothers, was performed by the Mount Sinai Mouse Genetics shared facility. All four ES clones gave rise to male chimeric mice (up to 90% chimeric based on far color; total number 16), which were mated with C57B1/6 females to obtain heterozygous offspring. For all four lines, germline transmission based on far color was observed in F1. Genomic DNA from F1 offspring was isolated, digested with BamHI, and analyzed by Southern hybridization with probe L. Fourteen females and 14 males, heterozygous for the SMC1β locus, were obtained. Heterozygous animals were mated to generate SMC1β^{-/-} progeny, or outcrossed with C57B1/6 to transfer the mutation into an inbred strain genetic background. The BamHI digested tail DNA. of F2 progeny of heterozygous animals was analyzed by Southern hybridization with probe L. Among the first 14 animals analyzed, two females and one male, homozygous for the. disrupted SMC1β locus, were found. The SMC1β^{-/-} mice appeared normal, suggesting that SMC1β is dispensable for survival and growth up to at least six weeks of age. At four weeks of age, the homozygous male and its wildtype and heterozygous siblings were sacrificed and testes were analysed. Testes from the wildtype and heterozygous animals were of similar size and weight; however, the testes from the SMC1β^{-/-} animal were half the size and weight. Immunofluorescence staining of testes sections with SMC1β monoclonal antibodies as described by Revenkova et al. Mol. Cell. Biol. 21:6984-6998, (2001) was carried out. As observed earlier in the wildtype mouse, antibodies stained compact chromosomal axes in prophase I nuclei. No staining specific for SMC1β was detected in the testis of −/− animals. Staining of nuclei with propidium iodide revealed that while the wild-type seminiferous tubules contained multiple layers of cells, the seminiferous tubules of the SMC1β-deficient animal had fewer cell layers and appeared comparatively empty. Homozygous SMC1β^{-/-} animals have appeared to be viable and have survived at least until four months of age now. The fertility of homozygotes is being evaluated; histological and cytological analysis of the reproductive organs is being carried out. Using these mice, it is possible to determine whether the knock-out of SMC1β alone leads to the loss of fertility.

Mouse genomic DNA was probed using Southern blotting analysis for the SMC1β gene. DNA from wildtype mice revealed a 9.6 kbp band representing the wildtype SMC1β allele. DNA from knock-out mice revealed a 6.3 kbp band representing the knockout allele. DNA from heterozygous mice revealed both alleles.

Immunofluorescence staining with anti SMC1β antibody (FITC-labeled; green) of wildtype or homozygous knockout mouse testes was carried out on testis sections from 4 week-old littermates. There was an absence of chromosomal staining in the seminiferous tubules of the knockout mouse.

Initial analysis of the testis and of spermatogenesis in the SMC1β knock-out mouse strain was performed. The testis in the four-week old SMC1β-deficient mouse is half the size and half the weight of a wildtype testis. With increasing age, this difference increased.

Testis morphology was examined by histological staining of testis tissue sections in four week-old littermates (wildtype, heterozygous, and knockout mice). The seminiferous tubules in the knock-out mice did not contain any mature spermatozoa. Cross-sections of the seminiferous tubules revealed no late stage spermatids, and the tubules were of reduced size. Spermatogenesis was blocked at prophase of meiosis I. Most cells appeared to be arrested at the leptotene/zygotene stage, with only a few cells that progressed up to the pachytene stage. There were no cells that progressed further than prophase I. Premeiotic cells, such as spermatogonia were unaffected, consistent with the onset of SMC1β expression in early meiosis (leptotene) in wildtype mice.

Periodic acid/Schiff staining of mouse testis sections from 11 week-old littermates at the same magnification showed seminiferous tubules of reduced size in knockout mice and the centers of the tubules of knockout mice were devoid of spermatids. The testis weights were 216 mg for heterozygotes, and 52 mg for knockout mice.

Testis sections from 11 week-old littermates were also assayed for apoptosis by the TUNEL assay, which visualizes broken DNA, a hallmark of apoptosis. In a normal, wildtype testis, one usually finds a small number of apoptotic cells, often premeiotic cells, occasionally meiotic cells. In the SMC1β knockout testis, the total number of apoptotic cells was only mildly increased. Most of the apoptotic cells were of the early meiotic types (leptotene/zygotene). This indicated that there is no large accumulation of dying or dead cells in the seminiferous tubules of knockout mice. Otherwise, the SMC1β-deficient testis may also be not as small as observed. Rather, arrested cells are removed from the system, probably through absorption by the somatic Sertoli cell layer. Sertoli cells are known to have some phagocytic function.

Studies of oogenesis in the SMC1β-deficient females are being performed. Because most known mouse mutants that affect meiosis have quite different phenotypes in females compared to males (Hunt and Hassold, Nature Rev. Genetics 2: 280-291, 2002), it is being investigated as to how female meiosis (oogenesis) is blocked in comparison with that seen for spermatogenesis. Also, female meiosis, due to the very long dictyate arrest at the end of prophase I, has its specifics and is in some ways more complicated than male meiosis. Initial studies have indicated that SMC1β-deficient females are sterile. To date, more studies have concentrated on spermatogenesis. However, SMC1β is a potential target for the inhibition of oogenesis as well.

Fertility tests are currently underway. Breeding pairs have been set up with either a SMC1β-deficient male mouse or a SMC1β-deficient female mouse, together with the respective wildtype partner. After many weeks of breeding, i.e. more than four reproductive cycles, no progeny have been obtained in any breeding with SMC1β-deficient mice.

Naturally-occurring deficiencies in a key meiosis-specific protein like SMC1β likely causes infertility. Infertility is a very common medical problem that affects about 10% of couples and has a significant familial component, with autosomal-recessive inheritance accounting for half of the male cases. Meiotic arrest is the most common type of sperm maturation arrest in men with non-obstructive azoospermia.

Thus, artificially disabling SMC1β may indeed turn into an advantage for regulating reproduction, i.e. for contraception. Conceptually it is very attractive to look for a target that is both highly important and highly specific for meiosis and SMC1β fulfills both requirements.

The data presented herein support the use of SMC1β as a target for a male contraceptive drug. Eliminating functional SMC1β should block male meiosis in humans and thus render the individuals sterile. This protein is also an attractive target because of it potential for contraceptive reversibility. Stopping drug treatment, which inhibits the expression of SMC1β, would allow for the quick reestablishment of fertility (one meiotic cycle at the most).

E. Regulation of SMC1β by Proteolysis

The present section provides a summary of what is known about the role(s) that protein phosphorylation and/or protein degradation play in the proteolysis and regulation of SMC1β. This summary may be helpful to the extent that it provides insight into the methods and compositions of the present invention.

In mitotic cells, the association of cohesin with the sister chromatids is terminated by targeted proteolysis of one subunit of cohesin, the Scc1/Rad21 protein (Uhlmann, Curr. Opin. Cell Biol. 13:754-761, 2001). In yeast, Scc1p is phosphorylated during metaphase of mitosis by the Polo-like kinase Cdc5, and then cleaved by an endopeptidase called separase (Uhlmann et al., Nature 400:37-42, 1999; Uhlmann et al., Cell 103:375-386, 2000). Separase itself gets activated by ubiquitin-dependent degradation of its inhibitor, securin. In higher eukaryotes, two distinct pathways are responsible for dissociation of cohesin from the chromosomes (Waizenegger et al., Cell 103:399-410, 2000). Most of cohesin, primarily at the chromosome arms, is removed by phosphorylation through a Polo-like kinase, early in mitosis, at the prophase/metaphase transition. The residual cohesin, however, which is mostly associated with the centromeric regions, is removed from the chromosomes by separase cleavage of Scc1, similar to the situation in yeast. Thus, in mitosis, a combined action of phosphorylation and specific protein degradation is required to dissociate cohesin from the right region at the right time.

Little is known about the mechanism by which dissociation of meiotic cohesin is regulated and achieved in mammalian cells. The dissociation of the SMC1β/SMC3 complex occurs in a two-step process: first, the complex is removed from the chromosome arms during metaphase I, and homologs, each consisting of two sister chromatids linked at their centromeres only, are segregated (Revenkova et al., Mol. Cell. Biol. 21: 6984-6998, 2001). In a second step, the remaining centromeric complex dissociates at the metaphase/anaphase II transition to allow segregation of the individual sister chromatids. Destruction of cohesion in the chromosome arms appears to be required for resolution of meiotic crossovers.

It is very likely, that phosphorylation and/or protein degradation play an important role in regulating the removal of SMC1β and its complex from meiotic chromosomes. Understanding this mechanism would fill a significant gap in our knowledge of the meiotic process and of regulation of SMC1β function. It is unlikely that association and dissociation of cohesins are regulated exclusively through Rec8, because Rec8 behaves differently from SMC1β and SMC3 (Eijpe et al., J. Cell Biol. 160:657-670,2003; and see below). As a first approach to address this question, a series of proteolysis assays were undertaken. In these assays, nuclear extracts from mouse testis were incubated under various conditions and investigated the intactness of SMC1β by immunoblotting.

The data indicated a progressive degradation of SMC1β with a 120-kDa intermediate being generated, but then quickly degraded as well. Addition of ATP increased the efficiency of the reaction. Interestingly, while the other cohesin subunits Rec8 or STAG3 (which may associate also with the SMC1α/SMC3 complex) were also degraded, their degradation was not enhanced by addition of ATP, but rather inhibited. This argues for an activity that is specific or specifically regulated for SMC1β. The ATP effect may indicate either an ATP-dependent activation of the protease (e.g. by phosphorylation or by an indirect mechanism such as ATP-dependent removal of an inhibitor), or an ATP-dependent targeting of SMC1β or degradation. For example, phosphorylation of SMC1β may trigger its degradation.

To begin testing the effect of phosphorylation, nuclear extracts were prepared in the presence or absence of general phosphatase inhibitors (1 mM o-vanadate, 10 mM β-glycerophosphate). If a phosphorylated SMC1β is more prone to degradation, inhibited dephosphorylation should stimulate degradation. The data showed that this is the case. The effect of phosphatase inhibition can either be direct—on SMC1β phosphorylation—or indirect, i.e. on maintaining an activated state of the protease or of a regulator of the protease. The mechanism of phosphatase inhibition and the identification of enzymes that are involved are currently being examined.

Speculating that the 120-kDa product seen in degradation assays is a true intermediate of SMC1β degradation, two fragments of SMC1β that span either one of the two putative regions of cleavage, positioned at about 30 kDa distance from each of the termini, were subcloned and expressed in E. coli. Thus, an N-terminal or a C-terminal polypeptide of 33 or 35 kDa, respectively, was expressed and purified. While the N-terminal fragment was purified to near homogeneity (100 mM imidazole fraction), there were one major and a few minor contaminants in the C-terminal fragment preparation. The putative region of cleavage was identified at the center of these polypeptides (N-terminal fragment: amino acids 174 to 420; C-terminal fragment: amino acids 820 to 1073). These proteins were used in cleavage inhibition assays. The data indicated that increasing amounts of the N-terminal protein, but not of the C-terminal protein, inhibited the degradation of SMC1β. Experiments are being repeated with more highly purified material, even though degradation was not affected by the C-terminal preparation. Thus, there is early evidence for a cleavage site within the N-terminal region between amino acids 174 and 420, and the precise cleavage site is being determined.

Analyzing the amino acid sequence within that N-terminal region for known protease cleavage sites (peptide cutter program; The ExPASy (Expert Protein Analysis System) proteomics server of the Swiss Institute of Bioinformatics, Geneva, SWITZERLAND) revealed a large number of sites for enzymes like trypsin, thermolysin, pepsin and that like, but only one site for the enzyme proline-endopeptidase (PEP). This site is at the center of the region. In addition, there are three cleavage sites for separase close together at the very C-terminal end of this region, making it perhaps somewhat less likely to be cleaved by separase (because the resulting SMC1β fragment would only be about 103 kDa, and would have to run abnormally high to appear as a 120-kDa band). The full-length SMC1β sequence demonstrated several more sites for each of these two enzymes, including a separase site in the C-terminal region, which, however, does not act as an inhibitor. Consensus sequences for both sites are not very restrictive (H/K/R-P-X [notP] for PEP, and S/D/E/I/G-X-E/D-X-X-R for separase). Based on these studies, it is very plausible that a cleavage signal like phosphorylation is specifying the actual cleavage site. In vitro, most members of the PEP family, however, cleave only short oligopeptides, although the crystal structure may accommodate larger substrates (Polgar, Cell. Mol. Life Sci. 59:349-362, 2002). In vivo substrates of PEP are largely unknown and likely relatively small peptides (e.g. peptide hormones), but a role for PEP in the meiosis and maturation of sperm was proposed based on its expression profile and results of inhibition experiments (Kimura et al., 2002). Thus, a role for PEP in the cleavage of SMC1β seems not very likely, but cannot be entirely ruled out. In light of its known function in regulating mitotic cohesin, a role for separase appears more plausible.

F. Assaying for Other Modulators of SMC1β Polypeptide Activity

Particular aspects of the present invention contemplate the modulation of SMC1β polypeptide expression or activity. In particular, methods of identifying modulators of SMC1β polypeptide expression or activity are provided in further detail herein below.

In some situations, it may be desirable to identify molecules that are modulators, i.e., agonists or antagonists, of the expression or activity of SMC1β polypeptide. Natural or synthetic molecules that modulate SMC1β polypeptide maybe identified using one or more screening assays, such as those described herein. Such molecules may be administered either in an ex vivo manner, or in an in vivo manner by injection, or by oral delivery, implantation device, or the like. “Test molecule(s)” refers to the molecule(s) that is/are under evaluation for the ability to modulate (i.e., increase or decrease) the activity of a SMC1β polypeptide. Most commonly, a test molecule will interact directly with a SMC1β polypeptide. However, it is also contemplated that a test molecule may also modulate SMC1β polypeptide activity indirectly, such as by affecting SMC1β gene expression, or by binding to a SMC1β binding partner (e.g., receptor, co-factor, or ligand). In one embodiment, a test molecule will bind to a SMC1β polypeptide with an affinity constant of at least about 10⁻⁶ M, preferably about 10⁻⁸ M, more preferably about 10⁻⁹ M, and even more preferably about 10⁻¹⁰ M.

Methods for identifying compounds which interact with SMC1β polypeptides are encompassed by the present invention. In certain embodiments, an SMC1β polypeptide is incubated with a test molecule under conditions which permit the interaction of the test molecule with a SMC1β polypeptide, and the extent of the interaction can be measured. The test molecule(s) can be screened in a substantially purified form or in a crude mixture.

In certain embodiments, a SMC1β polypeptide agonist or antagonist may be a protein, peptide, carbohydrate, lipid, or small molecular weight molecule which interacts with SMC1β polypeptide to regulate its expression or activity. Molecules which regulate SMC1β polypeptide expression or activity include nucleic acids which are complementary to nucleic acid encoding a SMC1β polypeptide, or are complementary to nucleic acids sequences which direct or control the expression or activity of SMC1β polypeptide, and which act as anti-sense regulators of expression or activity.

Once a set of test molecules has been identified as interacting with a SMC1β polypeptide, the molecules may be further evaluated for their ability to increase or decrease SMC1β polypeptide activity. The measurement of the interaction of test molecules with SMC1β polypeptides maybe carried out in several formats, including cell-based binding assays, membrane binding assays, solution-phase assays and immunoassays. In general, test molecules are incubated with a SMC1β polypeptide for a specified period of time, and SMC1β polypeptide activity is determined by one or more assays for measuring biological activity.

The interaction of test molecules with SMC1β polypeptides may also be assayed directly using polyclonal or monoclonal antibodies in an immunoassay. Alternatively, modified forms of SMC1β polypeptides containing epitope tags as described herein may be used in immunoassays.

In the event that SMC1β polypeptides display biological activity through an interaction with a binding partner (e.g., a receptor, a ligand or a co-factor), a variety of in vitro assays may be used to measure the binding of a SMC1β polypeptide to the corresponding binding partner (such as a selective binding agent, receptor, ligand, or co-factor). These assays may be used to screen test molecules for their ability to increase or decrease the rate and/or the extent of binding of a SMC1β polypeptide to its binding partner. In one assay, a SMC1β polypeptide is immobilized in the wells of a microtiter plate. Radiolabeled SMC1β binding partner (for example, iodinated SMC1β binding partner) and the test molecule(s) can then be added either one at a time (in either order) or simultaneously to the wells. After incubation, the wells can be washed and counted using a scintillation counter, for radioactivity to determine the extent to which the binding partner bound to SMC1β polypeptide. Typically, the molecules will be tested over a range of concentrations, and a series of control wells lacking one or more elements of the test assays can be used for accuracy in the evaluation of the results. An alternative to this method involves reversing the “positions” of the proteins, i.e., immobilizing SMC1β binding partner to the microtiter plate wells, incubating with the test molecule and radiolabeled SMC1β polypeptide, and determining the extent of SMC1β polypeptide binding. See, for example, Chapter 18, Current Protocols in Molecular Biology, Ausubel et al., eds., John Wiley & Sons, New York, N.Y. (1995).

As an alternative to radiolabelling, a SMC1β polypeptide or its binding partner may be conjugated to biotin and the presence of biotinylated protein can then be detected using streptavidin linked to an enzyme, such as horseradish peroxidase (HRP) or alkaline phosphatase (AP), that can be detected colorimetrically, or by fluorescent tagging of streptavidin. An antibody directed to a SMC1β polypeptide or to a SMC1β binding partner and conjugated to biotin may also be used and can be detected after incubation with enzyme-linked streptavidin linked to AP or HRP.

A SMC1β polypeptide or a SMC1β like binding partner can also be immobilized by attachment to agarose beads, acrylic beads or other types of such inert solid phase substrates. The substrate-protein complex can be placed in a solution containing the complementary protein and the test compound. After incubation, the beads can be precipitated by centrifugation, and the amount of binding between a SMC1β polypeptide and its binding partner can be assessed using the methods described herein. Alternatively, the substrate-protein complex can be immobilized in a column, and the test molecule and complementary protein are passed through the column. The formation of a complex between a SMC1β polypeptide and its binding partner can then be assessed using any of the techniques set forth herein, i.e., radiolabelling, antibody binding or the like.

Another in vitro assay that is useful for identifying a test molecule which increases or decreases the formation of a complex between a SMC1β polypeptide and a SMC1β binding partner is a surface plasmon resonance detector system such as the BIAcore assay system (Pharmacia, Piscataway, N.J.). The BIAcore system may be carried out using the manufacturer's protocol. This assay involves the covalent binding of either SMC1β polypeptide or a SMC1β binding partner to a dextran-coated sensor chip which is located in a detector. The test compound and the other complementary protein can then be injected, either simultaneously or sequentially, into the chamber containing the sensor chip. The amount of complementary protein that binds can be assessed based on the change in molecular mass which is physically associated with the dextran-coated side of the sensor chip; the change in molecular mass can be measured by the detector system.

In some cases, it may be desirable to evaluate two or more test compounds together for their ability to increase or decrease the formation of a complex between a SMC1β polypeptide and a SMC1β binding partner. In these cases, the assays set forth herein can be readily modified by adding such additional test compound(s) either simultaneous with, or subsequent to, the first test compound. The remainder of the steps in the assay are set forth herein.

In vitro assays such as those described herein may be used advantageously to screen large numbers of compounds for effects on complex formation by SMC1β polypeptide and SMC1β binding partner. The assays maybe automated to screen compounds generated in phage display, synthetic peptide, and chemical synthesis libraries.

Compounds which increase or decrease the formation of a complex between a SMC1β polypeptide and a SMC1β binding partner may also be screened in cell culture using cells and cell lines expressing either SMC1β polypeptide or SMC1β binding partner. Cells and cell lines may be obtained from any mammal, but preferably will be from human or other primate, canine, or rodent sources. The binding of a SMC1β polypeptide to cells expressing SMC1β binding partner at the surface is evaluated in the presence or absence of test molecules, and the extent of binding may be determined by, for example, flow cytometry using a biotinylated antibody to a SMC1β binding partner. Cell culture assays can be used advantageously to further evaluate compounds that score positive in protein binding assays described herein.

Cell cultures can also be used to screen the impact of a drug candidate. For example, drug candidates may decrease or increase the expression or activity of the SMC1β gene. In certain embodiments, the amount of SMC1β polypeptide that is produced may be measured after exposure of the cell culture to the drug candidate. In certain embodiments, one may detect the actual impact of the drug candidate on the cell culture. For example, the overexpression of a particular gene may have a particular impact on the cell culture. In such cases, one may test a drug candidate's ability to increase or decrease the expression of the gene or its ability to prevent or inhibit a particular impact on the cell culture. In other examples, the production of a particular metabolic product such as a fragment of a polypeptide, may result in, or be associated with, a disease or pathological condition. In such cases, one may test a drug candidate's ability to decrease the production of such a metabolic product in a cell culture.

A yeast two hybrid system (Chien et al., Proc. Natl. Acad. Sci. USA, 88:9578-9583, 1991) can be used to identify novel polypeptides that bind to, or interact with, SMC1β polypeptides. As an example, a yeast-two hybrid bait construct can be generated in a vector (such as the pAS2-1 from Clontech) which encodes a yeast GAL4-DNA binding domain fused to the SMC1β polynucleotide. This bait construct may be used to screen human cDNA libraries wherein the cDNA library sequences are fused to GAL4 activation domains. Positive interactions will result in the activation of a reporter gene such as β-Gal. Positive clones emerging from the screening may be characterized further to identify interacting proteins.

G. Therapeutic Uses of SMC1β

Methods for the treatment of infertility and the induction of fertility using SMC1β pharmaceutical compositions of the invention are within the scope of the present invention. More specifically, methods of the present invention contemplate the use of SMC1β pharmaceutical compositions in the regulation or dysregulation of gametogenesis, which encompasses the processes of spermatogenesis and oogenesis. To understand how such compositions might affect these processes, it is important to understand some aspects of spermatogenesis and oogenesis as set out below.

1. Spermatogenesis

The seminiferous tubules of the testis contain all the germ cells at various stages of maturation and their supporting Sertoli cells, which account for 85-90% of the testicular volume. Sertoli cells are a fixed-population of non-dividing support cells. Sertoli cells rest on the basement membrane of the seminiferous tubules and are linked by tight junctions. These tight junctions coupled with the close approximation of the myoid cells of the peritubular contractile cell layers serve to form the blood-testis barrier. This barrier provides a unique microenvironment that facilitates spermatogenesis and maintains these germ cells in an immunologically privileged location. This isolation is important because spermatozoa are produced during puberty, long after the period of self-recognition by the immune system. If these developing spermatozoa were not immunologically protected, they would be recognized as foreign and attacked by the body's immune system. Sertoli cells appear to be involved with the nourishment of developing germ cells as well as the phagocytosis of damaged cells. Spermatogonia and young spermatocytes are lower down in the basal compartment of the seminiferous tubule, whereas mature spermatocytes and spermatids are sequestered higher up in the adluminal compartment.

Germ cells or spermatogenic cells are arranged in an orderly manner from the basement membrane up to the lumen. Spermatogonia lie directly on the basement membrane, and next in order, progressing up to the lumen, are found the primary spermatocytes, secondary spermatocytes and spermatids. There are 13 different germ cells representing different stages in the developmental process.

Spermatogenesis is a complex process whereby primitive stem cells or spermatogonia, either divide to reproduce themselves for stem cell renewal or they divide to produce daughter cells that will later become spermatocytes. The spermatocytes eventually divide and give rise to mature cell lines that eventually give rise to spermatids. The spermatids then undergo a transformation into spermatozoa. This transformation includes nuclear condensation, acrosome formation, loss of most of the cytoplasm, development of a tail and arrangement of the mitochondria into the middle piece of the sperm which basically becomes the engine room to power the tail. Groups of germ cells tend to develop and pass through spermatogenesis together; this sequence of developing germ cells is called a generation. Generations of germ cells are basically in the same stage of development. There are six stages of seminiferous epithelium development in the human male. The progression from stage one through stage six constitutes one cycle. In humans the duration of each cycle is approximately 16 days and 4.6 cycles are required for a mature sperm to develop from early spermatogonia. Therefore, the duration of the entire spermatogenic cycle in humans is 74 days (4.6 cycles×16 days/cycle=74 days).

2. Oogenesis

Ovaries are egg-producing organs that hold between 200,000 and 400,000 follicles (from folliculus, meaning “sack” in Latin). These cellular sacks contain the materials needed to produce ripened eggs, or ova. In mammals, the ovary is the female gonad responsible for the differentiation and release of a mature oocyte for fertilization and successful propagation of the species. Equally important, the ovary is an endocrine organ that produces steroids to allow the development of female secondary sexual characteristics and support pregnancy. The outermost layer covering the ovary consists of germinal epithelium. Directly underneath the germinal epithelium there is a layer of dense connective tissue known as the tunica albuginea. The ovarian follicles, in conjunction with surrounding fibroblasts, collagen and elastic fibers, form the ovarian cortex located under the tunica albuginea. The ovarian medulla contains the blood vessels, lymphatic vessels and the nervus terminals. The formation of a functional ovary depends on three major events taking place during early stages of gonadogenesis: the initiation of meiosis, the formation of follicles and the differentiation of steroid producing cells.

An ovarian follicle is a highly complex unit consisting of distinct cell types. The ovarian follicle is comprised of several layers of somatic cells surrounding a fluid-filled cavity “antrum” in which the oocyte surrounded by somatic cells is bathed. The fluid found in the antrum is known as follicular fluid. The follicle provides a microenvironment for oocyte growth and it is responsible for the production of hormones. The oocyte and its companion somatic cells comprising the follicular unit maintain close association throughout development from primordial to preovulatory stages. The walls of mature preovulatory follicles consist of membrana granulosa, theca interna and theca externa. Granulosa cells are cells of epithelial origin important for the growth and survival of the oocyte. Granulosa cells are not a homogeneous tissue but rather specialized subpopulations consisting of the corona radiata, cumulus cells, mural and antral granulosa cells. The cumulus cells are the subpopulation of ovarian granulosa cells that surrounds the oocyte. The cumulus cells in close contact with the oocyte are known as corona radiata. They are in close contact with the oocyte through cytoplasmic extensions across the zona pellucida.

Follicles can be classified as primordial follicles, preantral follicles (primary and secondary follicles), antral and preovulatory follicles. Folliculogenesis is the process responsible for the development of ovulatory follicles and the release of one or more mature oocytes at a fixed interval throughout the reproductive life of a female. Folliculogenesis is resumed after a long quiescent phase and involves sequential subcellular and molecular transformations by various components of the follicle. During postnatal life, ovarian follicles continue to grow, mature and either ovulate or regress. Follicles are recruited continuously until the original store is exhausted.

Oocytes present in the adult ovary develop from a definite number of primordial germ cells (PGC). Primordial germ cells proliferate during migration and have undergone six or more divisions by the time they colonize the future gonad. Once established in the developing ovary, the proliferating PGC begin to differentiate into oogonia. The oogonia are the stem cells that give rise to all the oocytes in the ovary. The population of oogonia goes through a predetermined, species-specific, number of mitotic cycles until the cells enter the prophase of meiosis and become oocytes. The prophase of meiosis is traditionally separated into five sequential stages: leptotene, zygotene, pachytene, diplotene and diakinesis. The leptotene stage is resumed by the end of an active period of pre-meiotic DNA synthesis. At the leptotene stage, each chromosome condense from its interphase conformation to produce a fine discrete thread. Each chromosome has replicated and consists of two sister chromatids. Zygotene starts as soon as the synapsis or intimate pairing of homologous chromosomes is initiated. Each gene is brought in close juxtaposition with its homologous gene on the opposite chromosome. Each chromosome pair is usually called a bivalent, but each homologous chromosome consists of two sister chromatids or tetrad. The cells are said to have entered the pachytene stage of prophase as soon as synapsis is completed. The synaptonemal complexes are structures that hold the two homologous chromatids together. The maternal and paternal chromatids undergo homologous recombination to allow the exchanges or crossovers between two nonsister chromatids. When the oocyte reaches the pachytene stage, it becomes enclosed in a follicle. The diplotene stage starts with the unpairing of homologous chromosomes in each bivalent after the crossovers are completed. The bivalent remains joined by one or more chiasmata (crossover-sites). The chromosomes take a diffused aspect that permits a period of active RNA synthesis. The nucleus of diplotene oocytes is known as germinal vesicle (GV). The oocyte at the GV stage is a diploid cell (2n), which has twice the normal amount of DNA since the first meiotic prophase is stopped at diplotene since the early post-natal period. The oocytes are considered to be in meiotic arrest. Oocytes remain in meiotic arrest for many months or even years. The mammalian ovary has only a finite supply of oocytes.

The oogonia enlarge and initiate meiosis independently of any endocrine stimulation to form primary oocytes. The primary oocyte becomes arrested at the pachytene or dictyate stage of the first meiotic prophase. The oocyte and the follicular cells are interdependent. The oocyte requires the presence of granulosa cells to grow and survive. Pregranulosa cells rest on a delicate basement membrane opposite the stromal cells that give origin to the theca cells.

Primordial follicles are the fundamental developmental unit of the mammalian ovary. The store of primordial follicles is not renewable and serves the entire reproductive life span of the adult. As the follicles and oocytes start to grow, they move deeper into the cortex of the ovary. The follicle grows out through the cortex as the antrum develops and becomes visible on the surface of the ovary. The mechanism of activation of primordial follicles and oocytes remains unknown. The number of primordial follicles is determined during early life and most of them remain in a resting state. Before and throughout the reproductive life of the female a number of these primordial follicles leave the resting state and start to grow. The follicles develop to the antral stage and most of them undergo atresia; however, some of these follicles are rescued to reach the preovulatory stage. The end of the reproductive life occurs when the pool of resting primordial follicles is exhausted.

Other diseases or disorders caused or mediated by undesirable levels of SMC1β polypeptide are also encompassed within the therapeutic and diagnostic utilities that are part of the invention. Such diseases or disorders include, but are not limited to, infertility (as discussed herein), a pathological condition, and a nondisjunction syndrome. By way of illustration, such undesirable levels of SMC1β polypeptide include excessively elevated levels and sub-normal levels of SMC1β.

H. Selective Binding Agents of SMC1β

The present invention also provides selective binding agents of SMC1β for the diagnosis and/or treatment of a pathological condition resulting from abnormal levels of SMC1β. The diagnosis of such a condition comprises determining the presence or amount of expression or activity of the SMC1β polypeptide in a sample; and comparing the level of said polypeptide in a biological, tissue or cellular sample from either normal subjects or the subject at an earlier time, wherein susceptibility to a pathological condition is based on the presence or amount of expression or activity of the SMC1β nucleic acid or polypeptide.

As used herein, the term “selective binding agent” refers to a molecule which has specificity for one or more SMC1β polypeptides. Suitable selective binding agents include, but are not limited to, antibodies and derivatives thereof, polypeptides, and small molecules. Suitable selective binding agents may be prepared using methods known in the art. An exemplary SMC1β polypeptide selective binding agent of the present invention is capable of binding a certain portion of the SMC1β polypeptide thereby inhibiting the binding of the polypeptide to the SMC1β polypeptide receptor(s).

Selective binding agents such as antibodies and antibody fragments that bind SMC1β polypeptides are within the scope of the present invention. The antibodies may be polyclonal including monospecific polyclonal, monoclonal (MAbs), recombinant, chimeric, humanized such as CDR-grafted, human, single chain, and/or bispecific, as well as fragments, variants or derivatives thereof. Antibody fragments include those portions of the antibody which bind to an epitope on the SMC1β polypeptide. Examples of such fragments include Fab and F(ab′) fragments generated by enzymatic cleavage of full-length antibodies. Other binding fragments include those generated by recombinant DNA techniques, such as the expression of recombinant plasmids containing nucleic acid sequences encoding antibody variable regions.

Polyclonal antibodies directed toward a SMC1β polypeptide generally are produced in animals (e.g., rabbits or mice) by means of multiple subcutaneous or intraperitoneal injections of SMC1β polypeptide and an adjuvant. It may be useful to conjugate a SMC1β polypeptide to a carrier protein that is immunogenic in the species to be immunized, such as keyhole limpet heocyanin, serum, albumin, bovine thyroglobulin, or soybean trypsin inhibitor. Also, aggregating agents such as alum are used to enhance the immune response. After immunization, the animals are bled and the serum is assayed for anti-SMC1β polypeptide antibody titer.

Monoclonal antibodies directed toward SMC1β polypeptide are produced using any method which provides for the production of antibody molecules by continuous cell lines in culture. Examples of suitable methods for preparing monoclonal antibodies include the hybridoma methods of Kohler et al. (Nature, 256: 495-497, 1975) and the human B-cell hybridoma method (Kozbor et al., J. Immunol., 133: 3001-3005, 1984; Brodeur et al., Monoclonal Antibody Production Techniques and Applications, pp. 51-63, Marcel Dekker, Inc., New York, 1987). Also provided by the invention are hybridoma cell lines which produce monoclonal antibodies reactive with h2520-40 polypeptides.

The anti-SMC1β antibodies of the invention may be employed in any known assay method, such as competitive binding assays, direct and indirect sandwich assays, and immunoprecipitation assays (Sola, Monoclonal Antibodies: A Manual of Techniques, pp. 147-158 (CRC Press, Inc., 1987)) for the detection and quantitation of h2520-40 polypeptides. The antibodies will bind SMC1β polypeptides with an affinity which is appropriate for the assay method being employed.

For diagnostic applications, in certain embodiments, anti-SMC1β antibodies typically will be labeled with a detectable moiety. The detectable moiety can be any one which is capable of producing, either directly or indirectly, a detectable signal. For example, the detectable moiety may be a radioisotope, such as ³H, ¹⁴C, ³²P, ³⁵S, or ¹²⁵I, a fluorescent or chemiluminescent compound, such as fluorescein isothiocyanate, rhodamine, or luciferin; or an enzyme, such as alkaline phosphatase, β-galactosidase, or horseradish peroxidase (Bayer et al., Meth. Enz., 184: 138-163, 1990).

Competitive binding assays rely on the ability of a labeled standard (e.g., a SMC1β polypeptide, or an immunologically reactive portion thereof) to compete with the test sample analyte (a SMC1β polypeptide) for binding with a limited amount of anti-SMC1β antibody. The amount of a SMC1β polypeptide in the test sample is inversely proportional to the amount of standard that becomes bound to the antibodies. To facilitate determining the amount of standard that becomes bound, the antibodies typically are insolubilized before or after the competition, so that the standard and analyte that are bound to the antibodies may conveniently be separated from the standard and analyte which remain unbound.

Sandwich assays typically involve the use of two antibodies, each capable of binding to a different immunogenic portion, or epitope, of the protein to be detected and/or quantitated. In a sandwich assay, the test sample analyte is typically bound by a first antibody which is immobilized on a solid support, and thereafter a second antibody binds to the analyte, thus forming an insoluble three-part complex. See, e.g., U.S. Pat. No. 4,376,110. The second antibody may itself be labeled with a detectable moiety (direct sandwich assays) or may be measured using an anti-immunoglobulin antibody that is labeled with a detectable moiety (indirect sandwich assays). For example, one type of sandwich assay is an enzyme-linked immunosorbent assay (ELISA), in which case the detectable moiety is an enzyme.

The selective binding agents, including anti-SMC1β antibodies, are also useful for in vivo imaging. An antibody labeled with a detectable moiety may be administered to an animal, preferably into the bloodstream, and the presence and location of the labeled antibody in the host is assayed. The antibody may be labeled with any moiety that is detectable in an animal, whether by nuclear magnetic resonance, radiology, or other detection means known in the art.

Selective binding agents of the invention, including anti-SMC1β antibodies, may be used as therapeutics. These therapeutic agents are generally agonists or antagonists, in that they either enhance or reduce, respectively, at least one of the biological activities of a SMC1β polypeptide. In one embodiment, antagonist antibodies of the invention are antibodies or binding fragments thereof which are capable of specifically binding to a SMC1β polypeptide and which are capable of inhibiting or eliminating the functional activity of a SMC1β polypeptide in vivo or il vitro. In preferred embodiments, the selective binding agent, e.g., an antagonist antibody will inhibit the functional activity of a SMC1β polypeptide by at least about 50%, and preferably by at least about 80%. In another embodiment, the selective binging agent may be an antibody that is capable of interacting with a SMC1β binding partner (a ligand, co-factor, or receptor) thereby inhibiting or eliminating SMC1β activity in vitro or in vivo. Selective binding agents, including agonist and antagonist anti-SMC1β antibodies are identified by screening assays which are well known in the art.

The invention also contemplates to a kit comprising SMC1β selective binding agents (such as antibodies) and other reagents useful for detecting SMC1β levels in biological samples. Such reagents may include a secondary activity, a detectable label, blocking serum, positive and negative control samples, and detection reagents.

I. Pharmaceutical Compositions and Administration

Pharmaceutical compositions are within the scope of the present invention. Such SMC1β pharmaceutical compositions may comprise a therapeutically effective amount of a SMC1β polypeptide or an SMC1β nucleic acid molecule in admixture with a pharmaceutically or physiologically acceptable formulation agent selected for suitability with the mode of administration. Likewise, they may also comprise a contraceptively effective amount of one or more SMC1β selective binding agents in admixture with a pharmaceutically or physiologically acceptable formulation agent selected for suitability with the mode of administration. Such compositions may be administered in therapeutically or contraceptively effective amounts depending on the application.

Acceptable formulation materials preferably are nontoxic to recipients at the dosages and concentrations employed.

The pharmaceutical composition may contain formulation materials for modifying, maintaining or preserving, for example, the pH, osmolarity, viscosity, clarity, color, isotonicity, odor, sterility, stability, rate of dissolution or release, adsorption or penetration of the composition. Suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates, other organic acids); bulking agents (such as mannitol or glycine), chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides and other carbohydrates (such as glucose, mannose, or dextrins); proteins (such as serum albumin, gelatin or immunoglobulins); coloring; flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (sucrose or sorbitol); tonicity enhancing agents (such as alkali metal halides (preferably sodium or potassium chloride, mannitol sorbitol); delivery vehicles; diluents; excipients and/or pharmaceutical adjuvants. (Remington's Pharmaceutical Sciences, 18th Edition, A. R. Gennaro, ed., Mack Publishing Company, 1990).

The optimal pharmaceutical composition will be determined by one skilled in the art depending upon, for example, the intended route of administration, delivery format, and desired dosage. See for example, Remington's Pharmaceutical Sciences, supra. Such compositions may influence the physical state, stability, rate of in vivo release, and rate of in vivo clearance of the SMC1β molecule.

The primary vehicle or carrier in a pharmaceutical composition may be either aqueous or non-aqueous in nature. For example, a suitable vehicle or carrier may be water for injection, physiological saline solution or artificial cerebrospinal fluid, possibly supplemented with other materials common in compositions for parenteral administration. Neutral buffered saline or saline mixed with serum albumin are further exemplary vehicles. Other exemplary pharmaceutical compositions comprise Tris buffer of about pH 7.0-8.5, or acetate buffer of about pH 4.0-5.5, which may further include sorbitol of a suitable substitute therefor. In one embodiment of the present invention, SMC1β polypeptide compositions maybe prepared for storage by mixing the selected composition having the desired degree of purity with optional formulation agents (Remington's Pharmaceutical Sciences, supra) in the form of a lyophilized cake or an aqueous solution. Further, the SMC1β polypeptide product may be formulated as a lyophilizate using appropriate excipients such as sucrose.

The SMC1β pharmaceutical compositions can be selected for parenteral delivery. Alternatively, the compositions may be selected for inhalation or for delivery through the digestive tract, such as orally. The preparation of such pharmaceutically acceptable compositions is within the skill of the art.

The formulation components are present in concentrations that are acceptable to the site of administration. For example, buffers are used to maintain the composition at physiological pH or at slightly lower pH, typically within a pH range of from about 5 to about 8.

When parenteral administration is contemplated, the therapeutic compositions for use in this invention may be in the form of a pyrogen-free, parenterally acceptable aqueous solution comprising the desired SMC1β molecule in a pharmaceutically acceptable vehicle. A particularly suitable vehicle for parenteral injection is sterile distilled water in which a SMC1β molecule is formulated as a sterile, isotonic solution, properly preserved. Yet another preparation can involve the formulation of the desired molecule with an agent, such as injectable microspheres, bio-erodible particles, polymeric compounds (polylactic acid, polyglycolic acid), beads, or liposomes, that provides for the controlled or sustained release of the product which may then be delivered via a depot injection. Hyaluronic acid may also be used, and this may have the effect of promoting sustained duration in the circulation. Other suitable means for the introduction of the desired molecule include implantable drug delivery devices.

In one embodiment, a pharmaceutical composition may be formulated for inhalation. For example, a SMC1β like molecule may be formulated as a dry powder for inhalation. SMC1β polypeptide or SMC1β nucleic acid molecule inhalation solutions may also be formulated with a propellant for aerosol delivery. In yet another embodiment, solutions may be nebulized. Pulmonary administration is further described in PCT Application No. PCT/US94/001875, which describes pulmonary delivery of chemically modified proteins.

It is also contemplated that certain formulations may be administered orally. In one embodiment of the present invention, SMC1β molecules which are administered in this fashion can be formulated with or without those carriers customarily used in the compounding of solid dosage forms such as tablets and capsules. For example, a capsule may be designed to release the active portion of the formulation at the point in the gastrointestinal tract when bioavailability is maximized and pre-systemic degradation is minimized. Additional agents can be included to facilitate absorption of the SMC1β molecule. Diluents, flavorings, low melting point waxes, vegetable oils, lubricants, suspending agents, tablet disintegrating agents, and binders may also be employed.

Another pharmaceutical composition may involve an effective quantity of SMC1β molecules in a mixture with non-toxic excipients which are suitable for the manufacture of tablets. By dissolving the tablets in sterile water, or other appropriate vehicle, solutions can be prepared in unit dose form. Suitable excipients include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc.

Additional SMC1β pharmaceutical compositions will be evident to those skilled in the art, including formulations involving SMC1β polypeptides in sustained- or controlled-delivery formulations. Techniques for formulating a variety of other sustained- or controlled-delivery means, such as liposome carriers, bio-erodible microparticles or porous beads and depot injections, are also known to those skilled in the art. See for example, PCT/US93/00829 which describes controlled release of porous polymeric microparticles for the delivery of pharmaceutical composition's. Additional examples of sustained-release preparations include semipermeable polymer matrices in the form of shaped articles, e.g. films, or microcapsules. Sustained release matrices may include polyesters, hydrogels, polylactides (U.S. Pat. No. 3,773,919, EP 58,481), copolymers of L-glutamic acid and gamma ethyl-L-glutamate (Sidman et al., Biopolymers, 22:547-556, 1983), poly (2-hydroxyethyl-methacrylate) (Langer et al., J. Biomed. Mater. Res., 15; 167-277, 1981; Langer et al., Chem. Tech. 12:98-105,1982), ethylene vinyl acetate (Langer et al., supra) or poly-D(−)-3-hydroxybutyric acid (EP 133,988). Sustained-release compositions also include liposomes, which can be prepared by any of several methods known in the art. See e.g., Eppstein et al., Proc. Natl. Acad. Sci. USA, 82:3688-3692, 1985; EP 36,676; EP 88,046; EP 143,949.

The SMC1β pharmaceutical composition to be used for in vivo administration typically must be sterile. This may be accomplished by filtration through sterile filtration membranes. Where the composition is lyophilized, sterilization using this method may be conducted either prior to or following lyophilization and reconstitution. The composition for parenteral administration may be stored in lyophilized form or in solution. In addition, parenteral compositions generally are placed into a container having a sterile access port, for example, an intravenous solution bag or vial having a stopper pierceable by a hypodermic injection needle.

Once the pharmaceutical composition has been formulated, it may be stored in sterile vials as a solution, suspension, gel, emulsion, solid, or a dehydrated or lyophilized powder. Such formulations may be stored either in a ready-to-use form or in a form (e.g., lyophilized) requiring reconstitution prior to administration.

In a specific embodiment, the present invention is directed to kits for producing a single-dose administration unit. The kits may each contain both a first container having a dried protein and a second container having an aqueous formulation. Also included within the scope of this invention are kits containing single and multi-chambered pre-filled syringes (e.g., liquid syringes and lyosyringes).

An effective amount of a SMC1β pharmaceutical composition to be employed therapeutically including, but not limited to, the treatment of infertility or the induction of infertility will depend, for example, upon the therapeutic context and objectives. One skilled in the art will appreciate that the appropriate dosage levels for treatment will thus vary depending, in part, upon the molecule delivered, the indication for which the SMC1β molecule is being used, the route of administration, and the size (body weight, body surface or organ size) and condition (the age and general health) of the patient. Accordingly, the clinician may titer the dosage and modify the route of administration to obtain the optimal therapeutic effect. A typical dosage may range from about 0.01 μg/kg to up to about 100 mg/kg or more, depending on the factors mentioned above. In other embodiments, the dosage may range from 0.1 mg/kg up to about 100 mg/kg; or 1 mg/kg up to about 100 mg/kg; or 5 mg/kg up to about 100 mg/kg.

The frequency of dosing will depend upon the pharmacokinetic parameters of the SMC1β molecule in the formulation used. Typically, a clinician will administer the composition until a dosage is reached that achieves the desired effect. The composition may therefore be administered as a single dose, or as two or more doses (which may or may not contain the same amount of the desired molecule) over time, or as a continuous infusion via implantation device or catheter. Further refinement of the appropriate dosage is routinely made by those of ordinary skill in the art and is within the ambit of tasks routinely performed by them. Appropriate dosages may be ascertained through use of appropriate dose-response data.

The route of administration of the pharmaceutical composition is in accord with known methods, e.g. orally, through injection by intravenous, intraperitoneal, intracerebral (intra-parenchymal), intracerebroventricular, intramuscular, intra-ocular, intraarterial, intraportal, or intralesional routes, by sustained release systems or by implantation devices. Where desired, the compositions may be administered by bolus injection or continuously by infusion, or by implantation device.

Alternatively or additionally, the composition may be administered locally via implantation of a membrane, sponge, or another appropriate material on to which the desired molecule has been absorbed or encapsulated. Where an implantation device is used, the device may be implanted into any suitable tissue or organ, and delivery of the desired molecule may be via diffusion, timed-release bolus, or continuous administration.

In some cases, it may be desirable to use SMC1β pharmaceutical compositions in an ex vivo manner. In such instances, cells, tissues, or organs that have been removed from the patient are exposed to SMC1β pharmaceutical compositions after which the cells, tissues and/or organs are subsequently implanted back into the patient.

In other cases, a SMC1β polypeptide can be delivered by implanting certain cells that have been genetically engineered, using methods such as those described herein, to express and secrete the polypeptide. Such cells may be animal or human cells, and may be autologous, heterologous, or xenogeneic. Optionally, the cells may be immortalized. In order to decrease the chance of an immunological response, the cells may be encapsulated to avoid infiltration of surrounding tissues. The encapsulation materials are typically biocompatible, semi-permeable polymeric enclosures or membranes that allow the release of the protein product(s) but prevent the destruction of the cells by the patient's immune system or by other detrimental factors from the surrounding tissues.

J. Gene Therapy

Additional embodiments of the present invention relate to cells and methods (e.g., homologous recombination and/or other recombinant production methods) for both the in vitro production of therapeutic polypeptides and for the production and delivery of therapeutic polypeptides by gene therapy or cell therapy. Homologous and other recombination methods may be used to modify a cell that contains a normally transcriptionally silent SMC1β gene, or an under expressed gene, and thereby produce a cell which expresses therapeutically efficacious amounts of SMC1β polypeptides.

Homologous recombination is a technique originally developed for targeting genes to induce or correct mutations in transcriptionally active genes (Kucherlapati, Prog. Nucleic Acid Res. Mol. Biol. 36:301-310, 1989). The basic technique was developed as a method for introducing specific mutations into specific regions of the mammalian genome (Thomas et al., Cell 44:419-428, 1986; Thomas et al., Cell 51:503-512, 1987; Doetschman et al., Proc. Natl. Acad. Sci. USA, 85:8583-8587, 1988) or to correct specific mutations within defective genes (Doetschman et al., Nature 330:576-578, 1987). Exemplary homologous recombination techniques are described in U.S. Pat. No. 5,272,071 (EP 9193051, EP Publication No. 505500; PCT/US90/07642, International Publication No. WO 91/09955).

Through homologous recombination, the DNA sequence to be inserted into the genome can be directed to a specific region of the gene of interest by attaching it to targeting DNA. The targeting DNA is a nucleotide sequence that is complementary (homologous) to a region of the genomic DNA. Small pieces of targeting DNA that are complementary to a specific region of the genome are put in contact with the parental strand during the DNA replication process. It is a general property of DNA that has been inserted into a cell to hybridize, and therefore, recombine with other pieces of endogenous DNA through shared homologous regions. If this complementary strand is attached to an oligonucleotide that contains a mutation or a different sequence or an additional nucleotide, it too is incorporated into the newly synthesized strand as a result of the recombination. As a result of the proofreading function, it is possible for the new sequence of DNA to serve as the template. Thus, the transferred DNA is incorporated into the genome.

Attached to these pieces of targeting DNA are regions of DNA which may interact with or control the expression of a SMC1β polypeptide, e.g., flanking sequences. For example, a promoter/enhancer element, a suppressor, or an exogenous transcription modulatory element is inserted in the genome of the intended host cell in proximity and orientation sufficient to influence the transcription of DNA encoding the desired SMC1β polypeptide. The control element controls a portion of the DNA present in the host cell genome. Thus, the expression of the desired SMC1β polypeptide maybe achieved, not by transfection of DNA that encodes the SMC1β gene itself, but rather by the use of targeting DNA (containing regions of homology with the endogenous gene of interest) coupled with DNA regulatory segments that provide the endogenous gene sequence with recognizable signals for transcription of a SMC1β polypeptide.

In an exemplary method, the expression of a desired targeted gene in a cell (i.e., a desired endogenous cellular gene) is altered via homologous recombination into the cellular genome at a preselected site, by the introduction of DNA which includes at least a regulatory sequence, an exon and a splice donor site. These components are introduced into the chromosomal (genomic) DNA in such a manner that this, in effect, results in the production of a new transcription unit (in which the regulatory sequence, the exon and the splice donor site present in the DNA construct are operatively linked to the endogenous gene). As a result of the introduction of these components into the chromosomal DNA, the expression of the desired endogenous gene is altered.

Altered gene expression, as described herein, encompasses activating (or causing to be expressed) a gene which is normally silent (unexpressed) in the cell as obtained, as well as increasing the expression of a gene which is not expressed at physiologically significant levels in the cell as obtained. The embodiments further encompass changing the pattern of regulation or induction such that it is different from the pattern of regulation or induction that occurs in the cell as obtained, and reducing (including eliminating) the expression of a gene which is expressed in the cell as obtained.

One method by which homologous recombination can be used to increase, or cause, SMC1β polypeptide production from a cell's endogenous SMC1β gene involves first using homologous recombination to place a recombination sequence from a site-specific recombination system (e.g., Cre/loxP, FLP/FRT) (Sauer et al., Current Opinion In Biotechnology 5:521-527, 1994; Sauer et al., Methods In Enzymology 225:890-900, 1993) upstream (that is, 5′ to) of the cell's endogenous genomic SMC1β polypeptide coding region. A plasmid containing a recombination site homologous to the site that was placed just upstream of the genomic SMC1β polypeptide coding region is introduced into the modified cell-line along with the appropriate recombinase enzyme. This recombinase causes the plasmid to integrate, via the plasmid's recombination site, into the recombination site located just upstream of the genomic SMC1β polypeptide coding region in the cell line (Baubonis et al., Nucleic Acids Res. 21:2025-2029, 1993; O'Gorman et al., Science 251:1351-1355, 1991). Any flanking sequences known to increase transcription (e.g., enhancer/promoter, intron, translational enhancer), if properly positioned in this plasmid, would integrate in such a manner as to create a new or modified transcriptional unit resulting in de novo or increased SMC1β polypeptide production from the cell's endogenous SMC1β gene.

A further method to use the cell line in which the site specific recombination sequence had been placed just upstream of the cell's endogenous genomic SMC1β polypeptide coding region is to use homologous recombination to introduce a second recombination site elsewhere in the cell line's genome. The appropriate recombinase enzyme is then introduced into the two-recombination-site cell line, causing a recombination event (deletion, inversion, translocation) (Sauer et al., Current Opinion In Biotechnology, supra, 1994; Sauer, Methods In Enzymology, supra, 1993) that would create a new or modified transcriptional unit resulting in de novo or increased SMC1β polypeptide production from the cell's endogenous SMC1β gene.

An additional approach for increasing, or causing, the expression of SMC1β polypeptide from a cell's endogenous SMC1β gene involves increasing, or causing, the expression of a gene or genes (e.g., transcription factors) and/or decreasing the expression of a gene or genes (e.g., transcriptional repressors) in a manner which results in de novo or increased SMC1β polypeptide production from the cell's endogenous SMC1β gene. This method includes the introduction of a non-naturally occurring polypeptide (e.g. a polypeptide comprising a site specific DNA binding domain fused to a transcriptional factor domain) into the cell such that de novo or increased SMC1β polypeptide production from the cell's endogenous SMC1β gene results.

The present invention further relates to DNA constructs useful in the method of altering expression of a target gene. In certain embodiments, the exemplary DNA constructs comprise: (a) one or more targeting sequences; (b) a regulatory sequence; (c) an exon; and (d) an unpaired splice-donor site. The targeting sequence in the DNA construct directs the integration of elements (a)-(d) into a target gene in a cell such that the elements (b)-(d) are operatively linked to sequences of the endogenous target gene. In another embodiment, the DNA constructs comprise: (a) one or more targeting sequences, (b) a 20 regulatory sequence, (c) an exon, (d) a splice-donor site, (e) an intron, and (f) a splice-acceptor site, wherein the targeting sequence directs the integration of elements (a)-(f) such that the elements of (b)-(f) are operatively linked to the endogenous gene. The targeting sequence is homologous to the preselected site in the cellular chromosomal DNA with which homologous recombination is to occur. In the construct, the exon is generally 3′ of the regulatory sequence and the splice-donor site is 3′ of the exon.

If the sequence of a particular gene is known, such as the nucleic acid sequence of SMC1β polypeptide presented herein, a piece of DNA that is complementary to a selected region of the gene can be synthesized or otherwise obtained, such as by appropriate restriction of the native DNA at specific recognition sites bounding the region of interest. This piece serves as a targeting sequence(s) upon insertion into the cell and will hybridize to its homologous region within the genome. If this hybridization occurs during DNA replication, this piece of DNA, and any additional sequence attached thereto, will act as an Okazaki fragment and will be incorporated into the newly synthesized daughter strand of DNA. The present invention, therefore, includes nucleotides encoding a SMC1β polypeptide, which nucleotides may be used as targeting sequences.

SMC1β polypeptide cell therapy, e.g., the implantation of cells producing SMC1β polypeptides, is also contemplated. This embodiment involves implanting cells capable of synthesizing and secreting a biologically active form of SMC1β polypeptide. Such SMC1β polypeptide-producing cells can be cells that are natural producers of SMC1β polypeptides or may be recombinant cells whose ability to produce SMC1β polypeptides has been augmented by transformation with a gene encoding the desired SMC1β polypeptide or with a gene augmenting the expression of SMC1β polypeptide. Such a modification may be accomplished by means of a vector suitable for delivering the gene as well as promoting its expression and secretion. In order to minimize a potential immunological reaction in patients being administered a SMC1β polypeptide, as may occur with the administration of a polypeptide of a foreign species, it is preferred that the natural cells producing SMC1β polypeptide be of human origin and produce human SMC1β polypeptide. Likewise, it is preferred that the recombinant cells producing SMC1β polypeptide be transformed with an expression vector containing a gene encoding a human SMC1β polypeptide.

Implanted cells may be encapsulated to avoid the infiltration of surrounding tissue. Human or non-human animal cells may be implanted in patients in biocompatible, semipermeable polymeric enclosures or membranes that allow the release of SMC1β polypeptide, but prevent the destruction of the cells by the patient's immune system or by other detrimental factors from the surrounding tissue. Alternatively, the patient's own cells, transformed to produce SMC1β polypeptides ex vivo, may be implanted directly into the patient without such encapsulation.

Techniques for the encapsulation of living cells are known in the art, and the preparation of the encapsulated cells and their implantation in patients may be routinely accomplished. For example, Baetge et al. (WO95/05452; PCT/US94/09299) describe membrane capsules containing genetically engineered cells for the effective delivery of biologically active molecules. The capsules are biocompatible and are easily retrievable. The capsules encapsulate cells transfected with recombinant DNA molecules comprising DNA sequences coding for biologically active molecules operatively linked to promoters that are not subject to down-regulation in vivo upon implantation into a mammalian host. The devices provide for delivery of the molecules from living cells to specific sites within a recipient. In addition, see U.S. Pat. Nos. 4,892,538, 5,011,472, and 5,106,627. A system for encapsulating living cells is described in PCT Application no. PCT/US91/00157 of Aebischer et al. See also, PCT Application No. PCT/US91/00155 of Aebischer et al., Winn et al., Exper. Neurol. 113: 322-329, 1991, Aebischer et al., Exper. Neurol. 111:269-275, 1991; and Tresco et al., ASAIO 38:17-23, 1992.

In vivo and in vitro gene therapy delivery of SMC1β polypeptides is also envisioned. One example of a gene therapy technique is to use the SMC1β gene (either genomic DNA, cDNA, and/or synthetic DNA) encoding a SMC1β polypeptide which may be operably linked to a constitutive or inducible promoter to form a “gene therapy DNA construct”. The promoter may be homologous or heterologous to the endogenous SMC1β gene, provided that it is active in the cell or tissue type into which the construct will be inserted. Other components of the gene therapy DNA construct may optionally include, DNA molecules designed for site-specific integration (e.g. endogenous sequences useful for homologous recombination), tissue-specific promoter, enhancer(s) or silencer(s), DNA molecules capable of providing a selective advantage over the parent cell, DNA molecules useful as labels to identify transformed cells, negative selection systems, cell specific binding agents (as, for example, for cell targeting), cell-specific internalization factors, and transcription factors to enhance expression by a vector as well as factors to enable vector manufacture.

A gene therapy DNA construct can then be introduced into cells (either ex vivo or in vivo) using viral or non-viral vectors. One means for introducing the gene therapy DNA construct is by means of viral vectors as described herein. Certain vectors, such as retroviral vectors, will deliver the DNA construct to the chromosomal DNA of the cells, and the gene can integrate into the chromosomal DNA. Other vectors will function as episomes, and the gene therapy DNA construct will remain in the cytoplasm.

In yet other embodiments, regulatory elements can be included for the controlled expression of the SMC1β gene in the target cell. Such elements are turned on in response to an appropriate effector. In this way, a therapeutic polypeptide can be expressed when desired. One conventional control means involves the use of small molecule dimerizers or rapalogs (as described in WO9641865 (PCT/US96/099486); WO9731898 (PCT/US97/03137) and WO9731899 (PCT/US95/03157) used to dimerize chimeric proteins which contain a small molecule-binding domain and a domain capable of initiating biological process, such as a DNA-binding protein or transcriptional activation protein. The dimerization of the proteins can be used to initiate transcription of the transgene.

An alternative regulation technology uses a method of storing proteins expressed from the gene of interest inside the cell as an aggregate or cluster. The gene of interest is expressed as a fusion protein that includes a conditional aggregation domain which results in the retention of the aggregated protein in the endoplasmic reticulum. The stored proteins are stable and inactive inside the cell. The proteins can be released, however, by administering a drug (e.g., small molecule ligand) that removes the conditional aggregation domain and thereby specifically breaks apart the aggregates or clusters so that the proteins may be secreted from the cell. See, Science 287:816-817, and 826-830 (2000).

Other suitable control means or gene switches include, but are not limited to, the following systems. Mifepristone (RU486) is used as a progesterone antagonist. The binding of a modified progesterone receptor ligand-binding domain to the progesterone antagonist activates transcription by forming a dimer of two transcription factors which then pass into the nucleus to bind DNA. The ligand-binding domain is modified to eliminate the ability of the receptor to bind to the natural ligand. The modified steroid hormone receptor system is further described in U.S. Pat. No. 5,364,791; WO9640911; and WO9710337.

Yet another control system use's ecdysone (a fruit fly steroid hormone) which binds to and activates an ecdysone receptor (cytoplasmic receptor). The receptor then translocates to the nucleus to bind a specific DNA response element (promoter from ecdysone-responsive gene). The ecdysone receptor includes a transactivation-domain/DNA-binding domain/ligand-binding domain to initiate transcription. The ecdysone system is further described in U.S. Pat. No. 5,514,578; WO9738117; WO9637609; and WO9303162.

Another control means uses a positive tetracycline-controllable transactivator. This system involves a mutated tet repressor protein DNA-binding domain (mutated tet R-4 amino acid changes which resulted in a reverse tetracycline-regulated transactivator protein, i.e., it binds to a tet operator in the presence of tetracycline) linked to a polypeptide which activates transcription. Such systems are described in U.S. Pat. Nos. 5,464,758; 5,650,298 and 5,654,168.

Additional expression control systems and nucleic acid constructs are described in U.S. Pat. Nos. 5,741,679 and 5,834,186, to Innovir Laboratories Inc.

In vivo gene therapy may be accomplished by introducing the gene encoding a SMC1β polypeptide into cells via local injection of a SMC1β nucleic acid molecule or by other appropriate viral or non-viral delivery vectors. (Hefti, Neurobiology 25:1418-1435, 1994). For example, a nucleic acid molecule encoding a SMC1β polypeptide may be contained in an adeno-associated virus (AAV) vector for delivery to the targeted cells (e.g., Johnson, International Publication No. WO95/34670; International Application No. PCT/US95/07178). The recombinant AAV genome typically contains AAV inverted terminal repeats flanking a DNA sequence encoding a SMC1β polypeptide operably linked to functional promoter and polyadenylation sequences.

Alternative suitable viral vectors include, but are not limited to, retrovirus, adenovirus, herpes simplex virus, lentivirus, hepatitis virus, parvovirus, papovavirus, poxvirus, alphavirus, coronavirus, rhabdovirus, paramyxovirus, and papilloma virus vectors. U.S. Pat. No. 5,672,344 describes an in vivo viral-mediated gene transfer system involving a recombinant neurotrophic HSV-1 vector. U.S. Pat. No. 5,399,346 provides examples of a process for providing a patient with a therapeutic protein by the delivery of human cells which have been treated in vitro to insert a DNA segment encoding a therapeutic protein. Additional methods and materials for the practice of gene therapy techniques are described in U.S. Pat. No. 5,631,236 involving adenoviral vectors; U.S. Pat. No. 5,672,510 involving retroviral vectors; and U.S. Pat. No. 5,635,399 involving retroviral vectors expressing cytolines;

Nonviral delivery methods include, but are not limited to, liposome-mediated transfer, naked DNA delivery (direct injection), receptor-mediated transfer (ligand-DNA complex), electroporation, calcium phosphate precipitation, and microparticle bombardment (e.g., gene gun). Gene therapy materials and methods may also include the use of inducible promoters, tissue-specific enhancer-promoters, DNA sequences designed for site-specific integration, DNA sequences capable of providing a selective advantage over the parent cell, labels to identify transformed cells, negative selection systems and expression control systems (safety measures), cell-specific binding agents (for cell targeting), cell-specific internalization factors, and transcription factors to enhance expression by a vector as well as methods of vector manufacture. Such additional methods and materials for the practice of gene therapy techniques are described in U.S. Pat. No. 4,970,154 involving electroporation techniques; WO96/40958 involving nuclear ligands; U.S. Pat. No. 5,679,559 describing a lipoprotein-containing system for gene delivery; U.S. Pat. No. 5,676,954 involving liposome carriers; U.S. Pat. No. 5,593,875 concerning methods for calcium phosphate transfection; and U.S. Pat. No. 4,945,050 wherein biologically active particles are propelled at cells at a speed whereby the particles penetrate the surface of the cells and become incorporated into the interior of the cells.

It is also contemplated that SMC1β gene therapy or cell therapy can further include the delivery of one or more additional polypeptide(s) in the same or a different cell(s). Such cells may be separately introduced into the patient, or the cells may be contained in a single implantable device, such as the encapsulating membrane described above, or the cells may be separately modified by means of viral vectors.

A means to increase endogenous SMC1β polypeptide expression in a cell via gene therapy is to insert one or more enhancer element(s) into the SMC1β polypeptide promoter, where the enhancer element(s) can serve to increase transcriptional activity of the SMC1β gene. The enhancer element(s) used will be selected based on the tissue in which one desires to activate the gene(s); enhancer elements known to confer promoter activation in that tissue will be selected. For example, if a gene encoding a SMC1β polypeptide is to be “turned on” in T-cells, the lck promoter enhancer element may be used. Here, the functional portion of the transcriptional element to be added may be inserted into a fragment of DNA containing the SMC1β polypeptide promoter (and optionally, inserted into a vector and/or 5′ and/or 3′ flanking sequence(s), etc.) using standard cloning techniques. This construct, known as a “homologous recombination construct”, can then be introduced into the desired cells either ex vivo or in vivo.

Gene therapy also can be used to decrease SMC1β polypeptide expression by modifying the nucleotide sequence of the endogenous promoter(s). Such modification is typically accomplished via homologous recombination methods. For example, a DNA molecule containing all or a portion of the promoter of the SMC1β gene(s) selected for inactivation can be engineered to remove and/or replace pieces of the promoter that regulate transcription. For example the TATA box and/or the binding site of a transcriptional activator of the promoter may be deleted using standard molecular, biology techniques; such deletion can inhibit promoter activity thereby repressing the transcription of the corresponding SMC1β gene. The deletion of the TATA box or the transcription activator binding site in the promoter may be accomplished by generating a DNA construct comprising all or the relevant portion of the SMC1β polypeptide promoter(s) (from the same or a related species as the SMC1β gene(s) to be regulated) in which one or more of the TATA box and/or transcriptional activator binding site nucleotides are mutated via substitution, deletion and/or insertion of one or more nucleotides. As a result, the TATA box and/or activator binding site has decreased activity or is rendered completely inactive. The construct will typically contain at least about 500 bases of DNA that correspond to the native (endogenous) 5′ and 3′ DNA sequences adjacent to the promoter segment that has been modified. The construct may be introduced into the appropriate cells (either ex vivo or in vivo) either directly or via a viral vector as described herein. Typically, the integration of the construct into the genomic DNA of the cells will be via homologous recombination, where the 5′ and 3′ DNA sequences in the promoter construct can serve to help integrate the modified promoter region via hybridization to the endogenous chromosomal DNA.

K. Diagnostic Methods

The present invention encompasses methods of diagnosing a pathological condition or a susceptibility to a pathological condition in a subject caused by or resulting from abnormal levels of SMC1β nucleic acid or polypeptide. In particular, this aspect of the invention provides a method of diagnosing such a condition comprising determining the presence or amount of expression or activity of the SMC1β polypeptide in a sample; and comparing the level of said polypeptide in a biological, tissue or cellular sample from either normal subjects or the subject at an earlier time, wherein susceptibility to a pathological condition is based on the presence or amount of expression or activity of the SMC1β nucleic acid or polypeptide.

The present invention further provides methods of diagnosing abnormal levels of SMC1β by providing a tissue sample from a person, and determining the loss or mutation of a SMC1β encoding nucleic acid in the cells of said tissue. In particular, this aspect of the invention provides a method of diagnosing such abnormal levels by nucleic acid hybridization.

Suitable hybridization conditions for the diagnostic methods are those conditions that allow the detection of gene expression from identifiable expression units such as genes. Preferred hybridization conditions are stringent hybridization conditions, such as hybridization at 42° C. in a solution (i.e., a hybridization solution) comprising 50% formamide, 1% SDS, 1 M NaCl, 10% dextran sulfate, and washing twice for 30 minutes at 60° C. in a wash solution comprising 0.1×SSC and 1% SDS. It is understood in the art that conditions of equivalent stringency can be achieved through variation of temperature and buffer, or salt concentration, as described in Ausubel, et al. (Eds.), Protocols in Molecular Biology, John Wiley & Sons (1994), pp. 6.0.3 to 6.4.10. Modifications in hybridization conditions can be empirically determined or precisely calculated based on the length and the percentage of guanosine/cytosine (GC) base pairing of the probe. The hybridization conditions can be calculated as described in Sambrook, et al., (Eds.), Molecular Cloning: A Laboratory Manual, Cold Spring Harbor Laboratory Press: Cold Spring Harbor, N.Y. (1989), pp. 9.47 to 9.51.

The sequence of the SMC1β cDNA can be used to generate probes to detect chromosome abnormalities in the SMC1β. These probes maybe generated from both the sense and antisense strands of double-stranded DNA. The term “SMC1β probe” refers to both genomic and cDNA probes derived from the SMC1β gene.

cDNA probes capable of detecting mutations in the SMC1β gene are particularly preferred. Part or all of the SMC1β cDNA sequence may be used to create a probe capable of detecting aberrant transcripts of SMC1β.

Using the probes of the present invention, several methods are available for detecting chromosome abnormalities in the SMC1β gene. Such methods include, for example, Polymerase Chain Reaction (PCR) technology, restriction fragment length analysis, and oligonucleotide hybridization using, for example, Southern and Northern blotting and in situ hybridization.

PCR technology is practiced routinely by those having ordinary skill in the art and its uses in diagnostics are well known and accepted. Methods for practicing PCR technology are disclosed in PCR Protocols: A Guide to Methods and Applications, Innis, M. A. et al., Eds., Academic Press, San Diego, Calif. 1990, and RT-PCR, Clontech Laboratories (1991), which are incorporated herein by reference. Applications of PCR technology are disclosed in Polymerase Chain Reaction, Erlich, H A. et al., Eds., Cold Spring Harbor Press, Cold Spring Harbor, N.Y. 1989, which is incorporated herein by reference.

PCR technology allows for the rapid generation of multiple copies of DNA sequences by providing 5′ and 3′ primers that hybridize to sequences present in a DNA molecule, and further providing free nucleotides and an enzyme which fills in the complementary bases to the DNA sequence between the primers with the free nucleotides to produce a complementary strand of DNA. The enzyme will fill in the complementary sequences between probes only if both the 5′ primer and 3′ primer hybridize to DNA sequences on the same strand of DNA.

Antisense oligonucleotides which hybridize to at least a portion of an aberrant transcript resulting from a mutation of the SMC1β gene are also contemplated by the present invention. The oligonucleotide may match the target region exactly or may contain several mismatches. Thus, molecules which bind competitively to RNA coded by, for example, the SMC1β gene, for example, are envisioned for therapeutics.

Small interfering RNA (siRNA), which reduce or disrupt the expression of SMC1β gene, are also contemplated by the present invention. The term “RNA interference” was first used by researchers studying C. elegans and describes a technique by which post-transcriptional gene silencing (PTGS) is induced by the direct introduction of double stranded RNA (dsRNA: a mixture of both sense and antisense strands). Injection of dsRNA into C. elegans resulted in much more efficient silencing than injection of either the sense or the antisense strands alone (Fire et al., Nature 391:806-811, 1998). Just a few molecules of dsRNA per cell is sufficient to completely silence the expression of the homologous gene. Furthermore, injection of dsRNA caused gene silencing in the first generation offspring of the C. elegans indicating that the gene silencing is inheritable (Fire et al., supra, 1998). Current models of PTGS indicate that short stretches of interfering dsRNAs (21-23 nucleotides; siRNA also known as “guide RNAs”) mediate PTGS. siRNAs are apparently produced by cleavage of dsRNA introduced directly or via a transgene or virus. These siRNAs may be amplified by an RNA-dependent RNA polymerase (RdRP) and are incorporated into the RNA-induced silencing complex (RISC), guiding the complex to the homologous endogenous mRNA, where the complex cleaves the transcript. Thus, siRNAs are nucleotides of a short length (typically 18-25 bases, preferably 19-23 bases in length) which incorporate into an RNA-induced silencing complex in order to guide the complex to homologous endogenous mRNA for cleavage and degradation of the transcript.

While most of the initial studies were performed in C. elegans, RNAi is gaining increasing recognition as a technique that may be used in mammalian cell. It is contemplated that RNAi, or gene silencing, will be particularly useful in the disruption of SMC1β expression, and this may be achieved in a tissue-specific manner where desired. By placing a gene fragment encoding the desired dsRNA behind an inducible or tissue-specific promoter, it should be possible to inactivate genes at a particular location within an organism or during a particular stage of development.

Variations on RNA interference (RNAi) technology is revolutionizing many approaches to experimental biology, complementing traditional genetic technologies, mimicking the effects of mutations in both cell cultures and in living animals. (McManus & Sharp, Nat. Rev. Genet. 3, 737-747 (2002)). RNAi has been used to elicit gene-specific silencing in cultured mammalian cells using 21-nucleotide siRNA duplexes (Elbashir et al., Nature, 411:494-498, 2001; Fire et al., supra, 1998; Hannon, Nature 418, 244-251, 2002). In the same cultured cell systems, transfection of longer stretches of dsRNA yielded considerable nonspecific silencing. Thus, RNAi has been demonstrated to be a feasible. technique for use in mammalian cells and could be used for assessing gene function in cultured cells and mammalian systems, as well as for development of gene-specific therapeutics. In particularly preferred embodiments, the siRNA molecule is between 20 and 25 oligonucleotides in length an is derived from an SMC1β sequence. Particularly preferred siRNA molecules are 21-23 bases in length.

The term “oligonucleotide” as used herein includes both ribonucleotides and deoxyribonucleotides, and includes molecules which may be long enough to be termed “polynucleotides.” Oligodeoxyribonucleotides are preferred since oligoribonucleotides are more susceptible to enzymatic attack by ribonucleotides than deoxyribonucleotides. It will also be understood that the bases, sugars or internucleotide linkages may be chemically modified by methods known in the art. Modifications may be made, for example, to improve stability and/or lipid solubility. For instance, it is known that enhanced lipid solubility and/or resistance to nuclease digestion results by substituting a methyl group or sulfur atom for a phosphate oxygen in the internucleotide phosphodiester linkage. The phosphorothioates, in particular, are stable to nuclease cleavage and soluble in lipid. Modified oligonucleotides are termed “derivatives.”

The oligonucleotides of the present invention may be synthesized by any of the known chemical oligonucleotide synthesis methods. See for example, Gait, M. J., ed. (1984), Oligonucleotide Synthesis (IRL, Oxford). In certain embodiments, t10 or more oligonucleotide probes may be arrayed in the form of a diagnostic chip or “microarray” for the analysis and expression of these genes in various cell types. Such a microarray could be used for measuring gene expression of SMC1β and preferably comprises distinct sequences derived from wild-type and mutant SMC1β.

L. Relatedness of Nucleic Acid Molecules and/or Polypeptides

It is understood that related nucleic acid molecules include allelic or splice variants of the nucleic acid molecule of SEQ ID NOS: 1 and 3, and include sequences which are complementary to any of the above nucleotide sequences. Related nucleic acid molecules also include a nucleotide sequence encoding a polypeptide comprising or consisting essentially of a substitution, modification, addition and/or deletion of one or more amino acid residues compared to the polypeptides in SEQ ID NOS: 2 or 4.

Fragments include molecules which encode a polypeptide of at least about 25 amino acid residues, or about 50, or about 75, or about 100, or greater than about 100 amino acid residues of the polypeptides of SEQ ID NOS: 2 and 4.

In addition, related SMC1β nucleic acid molecules include those molecules which comprise nucleotide sequences which hybridize under moderately or highly stringent conditions as defined herein with the fully complementary sequence of the nucleic acid molecules of SEQ ID NOS: 1 and 3, or of molecules encoding polypeptides, which polypeptides comprises the amino acid sequences as shown in SEQ ID NOS: 2 and 4, or of a nucleic acid fragment as defined herein, or of a nucleic acid fragment encoding a polypeptide as defined herein. Hybridization probes maybe prepared using the SMC1β sequences provided herein to screen cDNA, genomic or synthetic DNA libraries for related sequences. Regions of the DNA and/or amino acid sequence of SMC1β polypeptide that exhibit significant identity to known sequences are readily determined using sequence alignment algorithms as described herein and those regions may be used to design probes for screening.

The term “highly stringent conditions” refers to those conditions that are designed to permit hybridization of DNA strands whose sequences are highly complementary, and to exclude hybridization of significantly mismatched DNAs. Hybridization stringency is principally determined by temperature, ionic strength, and the concentration of denaturing agents such as formamide. Examples of “highly stringent conditions” for hybridization and washing are 0.015 M sodium chloride, 0.0015 M sodium citrate at 65-68° C. or 0.015 M sodium chloride, 0.0015 M sodium citrate, and 50% formamide at 42° C. See Sambrook, Fritsch & Maniatis, Molecular Cloning: A Laboratory Manual, 2nd Ed., Cold Spring Harbor Laboratory, (Cold Spring Harbor, N.Y. (1989) and Anderson et al., Nucleic Acid Hybridization: a Practical Approach, Ch. 4, IRL Press Limited (Oxford, England).

More stringent conditions (such as higher temperature, lower ionic strength, higher formamide, or other denaturing agent) may also be used; however, the rate of hybridization will be affected. Other agents may be included in the hybridization and washing buffers for the purpose of reducing non-specific and/or background hybridization. Examples are 0.1% bovine serum albumin, 0.1% polyvinyl-pyrrolidone, 0.1% sodium pyrophosphate, 0.1% sodium dodecylsulfate (NaDodSO4 or SDS), ficoll, Denhardt's solution, sonicated salmon sperm DNA (or another non-complementary DNA), and dextran sulfate, although other suitable agents can also be used. The concentration and types of these additives can be changed without substantially affecting the stringency of the hybridization conditions. Hybridization experiments are usually carried out at pH 6.8-7.4; however, at typical ionic strength conditions, the rate of hybridization is nearly independent of pH. (See Anderson et al., Nucleic Acid Hybridization: a Practical Approach, Ch. 4, IRL Press Limited (Oxford, England)).

Factors affecting the stability of DNA duplex include base composition, length, and degree of base pair mismatch. Hybridization conditions can be adjusted by one skilled in the art in order to accommodate these variables and allow DNAs of different sequence relatedness to form hybrids. The melting temperature of a perfectly matched DNA duplex can be estimated by the following equation:
Tm(° C.)=81.5+16.6(log [Na+])+0.41(% G+C)−600/N−0.72(% formamide)

where N is the length of the duplex formed, [Na+] is the molar concentration of the sodium ion in the hybridization or washing solution, % G+C is the percentage of (guanine+cytosine) bases in the hybrid. For imperfectly matched hybrids, the melting temperature is reduced by approximately 1° C. for each 1% mismatch.

The term “moderately stringent conditions” refers to conditions under which a DNA duplex with a greater degree of base pair mismatching than could occur under “highly stringent conditions” is able to form. Examples of typical “moderately stringent conditions” are 0.015 M sodium chloride, 0.0015 M sodium citrate at 50-65° C. or 0.015 M sodium chloride, 0.0015 M sodium citrate, and 20% formamide at 37-50° C. By way of example, a “moderately stringent” condition of 50° C. in 0.015 M sodium ion will allow about a 21% mismatch.

It will be appreciated by those skilled in the art that there is no absolute distinction between “highly” and “moderately” stringent conditions. For example, at 0.015 M sodium ion (no formamide), the melting temperature of perfectly matched long DNA is about 71° C. With a wash at 65° C. (at the same ionic strength), this would allow for approximately a 6% mismatch. To capture more distantly related sequences, one skilled in the art can simply lower the temperature or raise the ionic strength.

A good estimate of the melting temperature in 1 M NaCl* for oligonucleotide probes up to about 20 nt is given by:
Tm=2° C. per A−T base pair+4° C. per G−C base pair

*The sodium ion concentration in 6× salt sodium citrate (SSC) is 1 M. See Suggs et al., Developmental Biology Using Purified Genes, p. 683, Brown and Fox (eds.) (1981).

High stringency washing conditions for oligonucleotides are usually at a temperature of 0-5° C. below the Tm of the oligonucleotide in 6× SSC, 0.1% SDS for longer oligonucleotides.

In another embodiment, related nucleic acid molecules comprise or consist of a nucleotide sequence that is about 70 percent (70%) identical to the nucleotide sequence as shown in SEQ ID NO: 1 or 3, or comprise or consist essentially of a nucleotide sequence encoding a polypeptide that is about 70 percent (70%) identical to the polypeptide set forth in SEQ ID NO: 2 or 4. In preferred embodiments, the nucleotide sequences are about 75 percent, or about 80 percent, or about 85 percent, or about 90 percent, or about 95, 96, 97, 98, or 99 percent identical to the nucleotide sequence as shown in SEQ ID NO: 1 or 3, or the nucleotide sequences encode a polypeptide that is about 75 percent, or about 80 percent, or about 85 percent, or about 90 percent, or about 95, 96, 97, 98, or 99 percent identical to the polypeptide sequence set forth in SEQ ID NO: 2 or 4. Differences in the nucleic acid sequence may result in conservative and/or non-conservative modifications of the amino acid sequence relative to the amino acid sequence of SEQ ID NO: 2 or 4.

Conservative modifications to the amino acid sequence of SEQ ID NO: 2 or 4 (and the corresponding modifications to the encoding nucleotides) will produce SMC1β polypeptides having functional and chemical characteristics similar to those of naturally occurring SMC1β polypeptide. In contrast, substantial modifications in the functional and/or chemical characteristics of SMC1β polypeptides may be accomplished by selecting substitutions in the amino acid sequence of SEQ ID NO: 2 or 4 that differ significantly in their effect on maintaining (a) the structure of the molecular backbone in the area of the substitution, for example, as a sheet or helical conformation, (b) the charge or hydrophobicity of the molecule at the target site, or (c) the bulk of the side chain.

For example, a “conservative amino acid substitution” may involve a substitution of a native amino acid residue with a normative residue such that there is little or no effect on the polarity or charge of the amino acid residue at that position. Furthermore, any native residue in the polypeptide may also be substituted with alanine, as has been previously described for “alanine scanning mutagenesis.”

Conservative amino acid substitutions also encompass non-naturally occurring amino acid residues which are typically incorporated by chemical peptide synthesis rather than by synthesis in biological systems. These include peptidomimetics, and other reversed or inverted forms of amino acid moieties.

Naturally occurring residues may be divided into classes based on common side chain properties:
1) hydrophobic: norleucine, Met, Ala, Val, Leu, Ile;
2) neutral hydrophilic: Cys, Ser, Thr, Asn, Gln;
3) acidic: Asp, Glu;
4) basic: His, Lys, Arg;
5) residues that influence chain orientation: Gly, Pro; and
6) aromatic: Ttp, Tyr, Phe.

For example, non-conservative substitutions may involve the exchange of a member of one of these classes for a member from another class. Such substituted residues maybe introduced into regions of the human SMC1β polypeptide that are homologous with non-human SMC1β polypeptide orthologs, or into the non-homologous regions of the molecule.

In making such changes, the hydropathic index of amino acids may be considered. Each amino acid has been assigned a hydropathic index on the basis of its hydrophobicity and charge characteristics. They are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is understood in the art byte et al., J. Mol. Biol., 157: 105-131, 1982). It is known that certain amino acids may be substituted for other amino acids having a similar hydropathic index or score and still retain a similar biological activity. In making changes based upon the hydropathic index, the substitution of amino acids whose hydropathic indices are within ±2 is preferred, those which are within il are particularly preferred, and those within ±0.5 are even more particularly preferred.

It is also understood in the art that the substitution of like amino acids can be made effectively on the basis of hydrophilicity, particularly where the biologically functionally equivalent protein or peptide thereby created is intended for use in immunological embodiments, as in the present case. The greatest local average hydrophilicity of a protein, as governed by the hydrophilicity of its adjacent amino acids, correlates with its immunogenicity and antigenicity, i.e., with a biological property of the protein.

The following hydrophilicity-values have been assigned to these amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5) and tryptophan (−3.4). In making changes based upon similar hydrophilicity values, the substitution of amino acids whose hydrophilicity values are within ±2 is preferred, those which are within ±1 are particularly preferred, and those within ±0.5 are even more particularly preferred. One may also identify epitopes from primary amino acid sequences on the basis of hydrophilicity. These regions are also referred to as “epitopic core regions.”

Desired amino acid substitutions (whether conservative or non-conservative) can be determined by those skilled in the art at the time such substitutions are desired. For example, amino acid substitutions can be used to identify important residues of the h2520-40 polypeptide, or to increase or decrease the affinity of the h2520-40 polypeptides described herein.

### M. EXAMPLES

The following examples present preferred embodiments and techniques, but are not intended to be limiting. Those of skill in the art will, in light of the present disclosure, appreciate that many changes can be made in the specific materials and methods which are disclosed and still obtain a like or similar result without departing from the spirit and scope of the invention.

### Example 1

### Control of SMC1β Expression

To determine the transcription start site for the SMC1β gene, primer extension of mRNA is carried out as described by Ausubel et al. John Wiley and Sons, Inc. (2003). For fall characterization of the 5′ region of SMC1β, the start site for the gene (GenBank Accession No. AK016311 (SEQ ID NO: 7)) that originates somewhere around the first exon of SMC1β and is transcribed in reverse orientation is determined. For primer annealing, total RNA either from mouse testis or liver (control tissue which does not express SMC1β) is used. Both transcripts are relatively abundant as they can be easily detected by Northern blotting. Therefore, primer extension products are abundant enough to be detected directly in the denaturing polyacrylamide gel.

Information on the position of transcription start sites allow for selection of DNA fragments for electrophoretic mobility shift assay described below. If the transcription start sites of the two genes are far apart, for example, if the start site for the gene (GenBank Accession No. AK016311 (SEQ ID NO: 7)) is located within the SMC1β coding sequence, subsequent studies would concentrate on the region surrounding the SMC1β transcription start site. If the start sites are close to each other, both are analyzed simultaneously. The GC-rich sequence between the two genes can cause reverse transcriptase to pause or terminate prematurely. Then products of intermediate size are visible in the gel. To prevent such artifacts, primer positions should be moved closer to the transcription start, so that the extension products are shorter than 100 nucleotides. High nucleotide concentration and elevated reaction temperature support an efficient reaction. As a positive control, the primer extension reaction with a primer for an unrelated testis-specific mRNA with a known transcription start site is used, for example, the germ-cell specific transcription factor, ALF (TFIIAalpha/beta-like factor; Xie et al, J. Biol. Chem. 277:17765-74, 2002).

To confirm the mapping results with a second approach, RNAse protection assay is used as described (Ausubel et al., 2003). For this assay, a commercially available kit (RiboQuant RPA kit, Pharmingen) is used. A series of probes specific for SMC1β upstream region are generated, and a probe for ribosomal protein L32 mRNA is used as a positive control. Samples of total RNA from testes and liver (negative control) are tested.

The same controls as used for primer extension assays are used, i.e. usage of RNA from mitotic cells, or analyzing a different meiosis-specific gene.

### Example 2

### Specific Protein Binding to the Promoter Region

To test specific binding of protein(s) to the promoter region, electrophoretic mobility shift assays (EMSA) are carried out with overlapping double-stranded synthetic oligonucleotides, approximately 30-35 bp long and spanning the region identified as a potential promoter by computer analysis. Oligonucleotides are designed to incorporate potential transcription factor binding sites detected by computer analysis. EMSA is performed according to published protocols (Akhmedov et al., J. Biol. Chem. 273:24088-24094, 1998; Akhmedov et al., J. Biol. Chem. 274:38216-38224, 1999). To identify factors specific for spermatogenesis, nuclear extracts, prepared either from mouse testis or liver, are used (Jessberger et al, Mol. Cell. Biol. 11:445-457, 1991; Jessberger et al., Journ. Biol. Chem. 26:15070-15079, 1993; Jessberger et al., Journ. Biol. Chem. 270:6788-6797, 1995; Jessberger et al., EMBO J. 15:4061-4068, 1996; Borggrefe et al., J. Biol. Chem. 273:17025-17035, 1998).

If testis-specific binding products are undetectable with the probes, proteins binding the diagnostic sequences might be present in a relatively low concentration. This could happen, for example, if a transcription factor is specific for a certain stage of meiosis, of which only relatively few cells are present in the total testis. To enrich nuclear extracts for stage-specific proteins, extracts are prepared from spermatocytes isolated by cell elutriation as described (Clifton et al., J. Endocrinol. 172:565-574, 2002; Borggrefe et al., J. Biol. Chem. 273:17025-17035, 1998) or the StaPut gradient method (Lam et al., Proc. Natl. Acad. Sci. USA 65:192-199, 1970).

To confirm binding specificity, competition experiments are performed. Unlabeled specific or non-specific and unrelated double-stranded oligonucleotides are used as competitors. If the sequence of oligonucleotides that bind testis-specific factor(s) corresponds to a consensus sequence for a known transcription factor, point mutations are then designed by the SequenceShaper program (Genomatix, Munich, GERMANY). This program predicts point mutations which delete defined binding sites without generating a new site or affecting other sites. Resulting oligonucleotides are used in competition experiments. For previously unknown binding sites, sets of random mutations are generated. To localize binding sites more precisely, DNase I footprinting analysis is performed as described (Choi et al., J. Biol. Chem. 266:20504-20511, 1991).

The data obtained by EMSA, supplemented by DNase I footprinting, provide the rationale for isolation of regulatory proteins by DNA affinity chromatography and for designing mutants to be tested in vivo.

### Example 3

### DNA Affinity Chromatography

Sequences within the SMC1β 5′ region that bind a testis-specific factor, as seen in EMSA and DNase footprinting assays, are used for purification of that binding activity from testis nuclear extracts. Standard DNA affinity chromatography, and an alternative method, oligonucleotide trapping (Gadgil et al., J. Chromatogr. A. 966:99-110, 2002), which is a modification of DNA affinity chromatography are used. For standard affinity chromatography, the double-stranded oligonucleotide is linked via an amino group coupled to one end to CNBr-sepharose beads. Nuclear extracts are loaded under conditions similar to the ones used in the EMSA experiments, and bound proteins are eluted with increasing salt concentration. In the alternative method, a column-attached single stranded oligonucleotide (AC)₅ is used to trap from the solution a double-stranded footprint oligonucleotide with single stranded (TG)₅ overhangs. First, the interaction between the binding protein and the footprint oligonucleotide is carried out in solution. The protein-DNA complex is then passed over an (AC)₅-Sepharose column where (TG)₅ overhangs anneal with high specificity to (AC)₅. The proteins are eluted from the column by high salt. This method permits protein binding at low DNA concentrations, therefore possibly reducing non-specific binding. Several competitors like heparin, single-stranded non-specific oligonucleotides, or non-specific double-stranded DNA, can be used to further reduce non-specific protein binding. For an additional control, nuclear extracts from mitotic cells or tissue are used. The specifically eluted proteins are separated on an SDS polyacrylamide gel and identified by microsequencing and/or mass spectrometry at the proteomics core facility at Mt. Sinai.

### Example 4

### Promoter Analysis with Reporter Genes

To analyze the SMC1β promoter activity in vivo, constructs containing the wildtype SMC1β promoter/enhancer region, it's subfragments, or it's mutated variants, fused to a reporter gene, are generated. Point mutations in putative regulatory factor binding sites are introduced by PCR using mutagenic primers, and the products are entirely sequenced (Sequencing core facility, Mt. Sinai). A 21-kbp BAC clone, subcloned 6 kbp, and sequenced 2100 bp has been obtained. Within that sequence, a theoretical promoter region has been identified, but enhancers my be located at an even further distance.

To determine the role of the predefined promoter sequences in SMC1β expression in spermatocytes, transgenic mice and/or a new spermatogonial cell culture system (Feng et al., Science 297:392-395, 2002) are used. For generation of transgenic mice, the expression constructs are injected by the Mt. Sinai Mouse Genetics Shared Facility. Verification of transgenic animals is performed by Southern analysis of tail DNA. At least two founders with different transgene insertion sites, and preferably a single copy of the transgene for each construct are selected. Transfection of spermatogonial cells and their subsequent release into meiosis is described below. Because the SMC1β promoter may be bidirectional, constructs with promoter elements in both orientations with respect to the reporter are made after having defined the critical regions.

The bacterial β-galactosidase (lacZ) gene is used as a reporter to allow for the analysis of transgene expression in situ in testis sections. This is done by a procedure allowing good conservation of the seminiferous epithelium structure as described by Sage et al (Mech. Dev. 80:29-39,1999). Briefly, testes are fixed in paraformaldehyde and incubated in X-gal solution for staining. After staining, the tissue is fixed in paraformaldehyde, embedded into a resin, and sections are made. For identification of cell types, sections are counterstained with hematoxylin (Mt. Sinai Histology Core Facility). Concentrations of paraformaldehyde and X-gal and duration of incubations are optimized if necessary.

Thus, the meiotic stage at which the transgene is expressed is determined by identification of the specific cell associations. Expression of the transgene is quantified by measuring β-galactosidase activity by a standard enzymatic assay and photometric read-out in cell extracts (Shaper et al, J. Biol. Chem. 269:25165-25171, 1994). This allows for the determination of tissue specificity of expression. A disadvantage of using lacZ as a reporter gene for the analysis of testis-specific expression is the presence of relatively high β-galactosidase activity in testis extracts (Shaper et al, J. Biol. Chem. 269:25165-25171, 1994). Nevertheless, lacZ has been successfully used as a reporter in studies of the activity of Hsp70-2 and Sycp1 promoter regions during spermatogenesis in transgenic mice (Dix et al., Dev. Biol. 174:310-321, 1996; Sage et al., Mech. Dev. 80:29-39, 1999). Alternatively, chloramphenicol acetyltransferase (CAT) or the luciferase activity assay are also used if the SMC1β promoter or its fragments confer a low level of transgene expression. Vectors containing CAT, the luciferase reporter gene, lacZ, and control vectors are all commercially available. CAT activity or luciferase activity is measured with commercial CAT enzyme assay systems or luciferase assay systems according to the manufacturer's protocols. In all experiments analyzing gene/enzyme expression in testes, corresponding control assays using somatic organs are included.

An additional way to analyze the specific stage of meiosis at which SMC1β regulatory elements become active is by comparing reporter protein activity in testis extracts from mice at different ages. The first wave of meiosis in male mice occurs at the stage of leptotene spermatocytes, which appears at about day 10 after birth. Pachytene cells appear at day 14. The activity of the reporter gene in juvenile mice testes is assayed between days 8 to 21 after birth.

### Example 5

### Methylation Patterns of CpG Sites in the SMC1β Promoter Region

The stretch of DNA 200 bp upstream of the SMC1β start codon contains 24 CpG sites. Initial evaluation of the methylation pattern of this region is done by digestion of the genomic DNA extracted from the testis and liver of adult mice with restriction endonucleases sensitive to methylation. Digested DNA is analyzed by Southern blotting with probes spanning the promoter region. Methylation-sensitive restriction endonucleases, BstUI, Hha I, and HpaII, are used, for example. The restriction endonuclease, MspI, is used as a control because it recognizes the same site as HpaII, but is insensitive to methylation. There are four BstUI sites, five HhaI sites, and one HpaII site located within the stretch of DNA 200 bp upstream of the SMC1β start codon. A probe for mitochondrial DNA, which is not methylated, is also used to verify that the digestion was complete.

To obtain data on the exact methylation pattern of the promoter region and to evaluate the degree of methylation for each CpG site, the bisulfite method (Clark et al., Nucleic Acids Res. 22:2990-2997, 1994) is used. Purified genomic DNA from somatic tissues and testes are treated with sodium bisulfite to convert the unmethylated cytosines to uracil residues. The treatment leaves methylated cytosines unaffected. The SMC1β promoter region is subsequently amplified by PCR with specific primers, and the products are cloned and sequenced. When the conversion by bisulfite is complete, all cytosine residues in the sequence will represent the cytosines methylated in the genome. As a control for complete conversion of unmethylated cytosines, plasmid DNA containing the promoter region is mixed with genomic DNA, treated with bisulfite, amplified with vector specific primers, and sequenced. A modification of the bisulfite method by Olek et al. (Nucleic Acids Res. 24:5064-5066, 1996) allows the resolution of methylation patterns using genomic DNA from less than 100 individual cells. It is useful to examine methylation of the SMC1β promoter in testicular cells at different stages of spermatogenesis. Germ cell populations are isolated by StaPut method as described (Lam et al, Proc Natl Acad Sci USA 65:192-199, 1970). Testes from immature mice are used to evaluate the methylation status of the promoter in spermatogonia.

To determine if methylation patterns of the SMC1β promoter differ in somatic and spermatogenic cells, a series of experiments are performed to examine if methylation affects SMC1β promoter activity. First, to determine if demethylation of the promoter activates trancription, cultured cells of established lines (for example, 293 cells, which do not have any detectable SMC1β transcription) or mouse embryonic fibroblasts are treated with 5-Aza-2′-deoxycytidine, non-methylatable cytosine analog, and transcription of SMC1β are assessed by RT-PCR. If transcription of SMC1β depends on demethylation, the non-methylated promoter constructs, introduced into cultured cells by plasmid transfection, will be transcriptionally active. Plasmids are then methylated in vitro by treatment with SssI methylase, and the gene activity of methylated and non-methylated constructs is then compared. If non-methylated promoter constructs are active in cell lines, they are used in transfection experiments to analyze the effects of deletions and point mutations. It is then determined if the SMC1β promoter fused to a reporter gene is methylated and silenced after stable integration and propagation in cultured cell lines. To determine if methylation affects binding of testis-specific factors, EMSA experiments with synthetic methylated oligonucleotides as competitors are repeated.

### Example 6

### Regulation of SMC1β Function

The involvement of targeted proteolysis in the regulation of SMC1β function has been suggested by preliminary data. Such proteolysis is not unprecedented for cohesin proteins (e.g. mitotic Scc1). Therefore, it is likely that for the proper regulation of SMC1β function, and therefore of meiotic progression, the protein must be removed from meiotic chromosomes at specific stages and from specific regions of the chromosomes. Because the dissociation is a two-step process as follows: 1) dissociation from the chromosome arms in metaphase I; and 2) dissociation from the centromeres at metaphase II, it must not only be highly controlled, but also be governed by stage- and chromosomal region-specific signals. Two mechanisms of control used in many biological systems for comparable processes, and for dissociation of mitotic cohesin from mitotic chromosomes, are protein degradation and/or protein phosphorylation.

There is evidence for both protein degradation and protein phosphorylation in the regulation of SMC1β function. To better understand the role that protein degradation plays in the regulation of SMC1β, SMC1β-degrading proteolytic activity assays are being . . repeated, but preliminary results have indicated that this activity: 1) has a certain specificity for SMC1β; 2) depends on ATP; 3) is significantly higher if phosphatases present in the extract are blocked; 4) generates a 120-kDa intermediate; and 5) is inhibited specifically by an N-terminal peptide of SMC1β. Experiments are also being undertaken to purify the protease that degrades SMC1β, determine the cleavage site(s) within SMC1β, define the role of phosphorylation in the cleavage reaction, and purify the respective kinase. In addition, the role of SMC1β degradation in vivo is being determined.

### Example 7

### Protease Purification

Analysis of the amino acid sequence of the inhibitory N-terminal region of SMC1β revealed a single cleavage site for proline endopeptidase (PEP), and clusters of three cleavage sites for separase. While, PEP is an unlikely enzyme to be responsible for SMC1β cleavage, PEP's involvement in SMC1β cleavage is being examined. In fact, both PEP and separase are being tested for a role in the cleavage of SMC1β. Specific inhibitors are available for both enzymes: Boc-Glu(NHO-Bz)-Pyr (Ki 30 nM inhibit human PEP; Calbiochem Inc.), and non-cleavable peptides inhibit separase (Waizenegger et al., Cell 103:399-410, 2000). These inhibitors are useful in cleavage assays in vitro containing testis nuclear extracts. These proteases are also being examined using in vivo assays.

If inhibition experiments provide evidence for a role for either PEP or separase in SMC1β cleavage, recombinant PEP (purified by standard Ni-column chromatography; Kimura et al., Zool. Sci. 19:93-102, 2002), or immunoprecipitated separase (shown to be active; antibodies are available from Dr. J. M. Peters, Vienna), is then used to analyze cleavage of immunoprecipitated SMC1β. If PEP or separase (or both) cleave the SMC1β protein, the N-terminal peptide, or a peptide that carries a mutated protease recognition site, is. then used as an inhibitor(s) in control experiments. While there are several sites for each of the enzymes in the full-length SMC1β, the site which generates the 120-kDa intermediate is being sought. If SMC1β is rapidly degraded, regardless of which inhibitor peptide is added, the protease is nonspecific or specific regulatory mechanisms are missing in the reaction, e.g. cleavage of a phosphorylated SMC1β. SMC1β is then prepared from extract treated with phosphatase or phosphatase-inhibitors and/or with ATP. If the N-terminal peptide, but not control peptides, inhibit the reaction, there is either no cleavage or delayed cleavage. However, if inhibition by the N-terminal peptide depends on its phosphorylation, or on co-factors, this system, based on individual purified proteins, will not show the inhibitory effect nor specificity. Ex vivo purification experiments are carried out as described herein. Any positive result in cleavage assays, using a recombinant, purified protease, is also verified by in vivo experiments discussed herein.

It is also possible that neither one of these two candidate proteases, PEP nor separase, cleaves SMC1β at the terminal site. SMC1β protease is then purified from testis nuclear extracts by a wide variety of established chromatographic and protein separation methods. These methods include gel filtration on various resins like Superdex75 and 200 for lower and medium molecular weight ranges and BioGelA15m and A50m for the high molecular weight ranges. These methods further include ion exchange chromatography on BioRex70, DEAE cellulose, MacroQ, and MacroS resins. They also include mixed affinity-ion exchange chromatography on heparin sepharose and a variety of dye-sepharose resins. Precise details of these chromatography procedures are known to one of skill in the art, and are described in the references cited herein.

Extracts are prepared by salt-extraction of isolated nuclei (Jessberger et al., Mol. Cell. Biol. 11:445-457, 1991; Jessberger et al., Journ. Biol. Chem. 268:15070-15079, 1993, Jessberger et al., EMBO J. 15:4061-4068, 1996; Borggrefe et al., J. Biol. Chem. 273:17025-17035, 1998), and as described in initial cleavage assays. The stringency of extraction is varied, for example, either by altering salt concentrations (usually in the range from 0.25 to 0.75 M KCl) or by varying the salt from KCl, to ammonium sulfate, or to NaCl to optimize the starting material. Other parameters during extraction and fractionation are varied to optimize the experiment by one of skill in the art, including the use of reducing or non-reducing buffers, the use of mild detergents, different pH, etc. However, a robust activity has been seen with extracts as prepared, and variations are limited except when necessary or deliberate.

The read-out is as described, i.e. immunoblot analysis of SMC1β degradation. Unlike that in total nuclear extracts, there is probably no SMC1β in more purified protease fractions, the SMC1β/SMC3 dimer, immunoprecipitated from testis extracts (a highly reproducible, efficient method; see Revenkova et al., Mol. Cell. Biol. 21:6984-6998, 2001) is added as a substrate. Experiments have shown that this substrate is cleaved in testis nuclear extracts (in addition to the endogenous protein). Alternatively, commercially available in vitro transcription/translation systems, that by radioactive labeling of SMC1β with S³⁵-methionine also are used to generate a sensitive read-out, even if low amounts of this relatively large protein are being synthesized. If two or more activities need to act together to specifically cleave SMC1β (e.g., if specific cleavage by just one protein fraction is not seen) fractions, or pools of fractions, are combined and further purified. As a further control, the inhibitory N-terminal fragment is used to ensure purification of the specific protease.

The protease is identified by mass spectrometry (proteomics core facility at Mt. Sinai). Identification of the protease allows for the study of SMC1β regulation. An in vitro system that uses immunoprecipitated SMC1β and recombinant or ex vivo purified protease is then established to study details of the mechanism and regulation of protease cleavage. This allows for better study of the inhibition of the protease or its activation. These cell-free experiments are carried out with the same controls and limitations as discussed herein for the other two candidate proteases. However, this system is more specific and complete, because any necessary co-factor(s) (additional fraction), are simultaneously purified. Experiments are then performed to analyze the role of the protease in meiosis in vivo.

### Example 8

### Identification of the SMC1β Protease Cleavage Site

In attempts to identify the protease cleavage site within SMC1β, N-terminal and C-terminal protein fragments of SMC1β were purified in increasing concentrations of imidazole in elution buffer and run on silver-stained SDS polyacrylamide gels. To assay degradation, increasing amounts of N- or C-terminal protein fragments were then added to a reaction containing extract incubated with and without competitor. Reactions were then separated eletrophoretically on SDS polyacrylamide gels, immunoblotted, and probed with anti-SMC1β antibody. The N-terminal 33-kDa fragment was identified as an inhibitor to protease cleavage. While this is likely competitive inhibition, it cannot be ruled out that this peptide inhibits a protease through a different mechanism which does not require it to be a generic cleavage substrate. Cleavage of the N-terminal fragment itself is currently being investigated.

Based on the hypothesis that the N-terminal region of SMC1β contains a cleavage site that is required for the subsequent total degradation of SMC1β, smaller fragments of the N-terminal region are being used to determine the size of potential cleavage products of that fragment, and thus minimize the inhibitory peptide. In analogy to many other protease cleavage recognition sites, a minimal peptide is expected to be not more than 30 amino acids in length. Such peptides are chemically synthesized, and a variety of mutants are made. Failure of such mutants to inhibit the cleavage reaction provides a good indication of the protease target site.

Having identified such a minimal inhibitory sequence, or even specific amino acids required for inhibition, an SMC1β protein that is mutated in these sites is created, either by deleting the entire minimal sequence, or by point mutating the essential amino acids. This mutant SMC1β is then expressed in an in vitro transcription/translation system, and tested by incubation in either an extract that has been depleted for SMC1β, or in a (semi-)purified protease fraction. The mutant protein is also tested in vivo as discussed herein.

### Example 9

### The Role of Phosphorylation

Results have shown that the presence of phosphatase inhibitors stimulates the cleavage reaction. Therefore, the addition of phosphatase to the reaction to inhibit the cleavage is being examined. It has also been shown that ATP stimulates SMC1β degradation. This effect may be explained in several ways, e.g. by activation of the proteasome, which in turn removes a protease inhibitor. However, it may also indicate activation of a kinase present in the testis nuclear extract that phosphorylates SMC1β. Experiments have shown that the N-terminal fragment of SMC1β is phosphorylated by incubation in the extract with γ-³²P-ATP.

The effect of phosphorylation may be either on SMC1β—marking it for degradation—or on the protease—activating the enzyme. The effect may even be indirect, e.g. activating an activator or inhibiting an inhibitor of the protease. Currently, either of these possibilities cannot be distinguished. However, the phosphorylation of SMC1β most likely stimulates, or is even required, for it's degradation. This hypothesis is based on analogies: analogy with Scc1, another cohesin subunit, that in mitosis becomes targeted for degradation by phosphorylation. This hypothesis is also based on the reported phosphorylation of SMC1α, the ubiquitous isoform of SMC1β, by ATM kinase (Yazdi et al., Genes Dev. 16:571-582, 2002; Kim et al., Genes & Dev. 16:560-570, 2002); the observation (by ³²P-labeling and two-dimensional gel electrophoresis of immunoprecipitated material) of phosphorylation of SMC1α and SMC3 in mitotic cells; and the observation of phosphorylation of the SMC1β N-terminal peptide in testis nuclear extracts.

Assuming that a phosphorylated SMC1β is a target for protein degradation, the identify of the respective kinase in the cleavage reaction is being investigated. Immunoprecipitated SMC1β is used as a substrate in standard kinase assays. γ-³²P-ATP is incubated under appropriate buffer conditions with the substrate and a protein fraction. Products are visualized by autoradiography after SDS gel electrophoresis. Controls include omission of the substrate, use of large numbers of fractions that are negative, and use of unrelated proteins as substrate. Activity is also examined in mitotic cells. However, the kinase does not necessarily have to be meiosis-specific, because, for example, its own activation pathway may be meiosis-specific.

Besides using the full-length SMC1β, fragments of SMC1β expressed in E. coli are also used. It has already been determined that the N-terminal, but not the C-terminal fragment, is phosphorylated by testis nuclear extracts. This observation is being confirmed in current experiments, but indicates that the N-terminal fragment is useful as a competitive inhibitor or as a substrate for kinase purification (including as an affinity substrate in affinity chromatography) for kinase reactions.

There are many possibilities as to why a recombinant, non-phosphorylated N-terminal fragment inhibits the protease reaction. For example, the peptide may just be bound, but not cleaved, if phosphorylation is required for cleavage. Also, the inhibitory effect may be seen only because of excess peptide, which is a weak substrate. Likewise, phosphorylation may be required only for targeting the full-length SMC1β, not for a small fragment.

Some known kinases are candidates for phosphorylating SMC1β. For example, ATM is known to phosphorylate SMC1α and is also required for meiosis. Furthermore, meiosis in ATM-deficient mice is disrupted at the pachytene stage (Barlow et al., Development 125:4007-4017, 1998). Heterozygous ATM-deficient mice are being bred; and chromosome spreads for the distribution of SMC1β in prophase I cells are being analyzed. A cell line that expresses a tagged ATM protein has been obtained (from Dr. Y. Shiloh), from which the kinase is immunoprecipitated and is used in kinase assays.

Another candidate kinase is the cyclinA1/Cdc2, which has been shown to be specifically expressed in male germ cells during late prophase I (Ravnik et al., Dev. Biol. 207:408-418, 1999). SMC1β expression in meiotic cells from cyclin A1-deficient mice (provided by Dr. Debra Wolgemuth, Columbia University) is being studied with the use of an anti-SMC1β antibody. Spermatogenesis in these mice has been shown to stop at metaphase I (Liu et al., Dev Biol. 224:388-400, 2000).

Having purified a kinase that phosphorylates SMC1β, the specific phosphorylation site is determined. If the reaction is highly efficient, direct analysis by mass spectrometry is performed. Alternatively, individual domains of SMC1β, expressed in E. coli, are used as kinase substrates to identify and minimize the region of phosphorylation.

The very N-terminus, the protease-inhibitory N-terminal fragment, the hinge domain, the C-terminal fragment used in inhibition experiments, and the very C-terminus have been expressed and purified. Together these fragments account for 86% of the entire SMC1β protein. Should the phosphorylation site reside in the remaining sequences, the respective protein region is expressed and purified. After reducing the phosphorylation region to a few hundred amino acids or less, standard methods, like mass spectrometry or peptide cleavage/thin layer chromatography, are used to identify the phosphorylated residue(s).

### Example 10

### Functional Testing with SMC1β In Vivo

Results obtained by molecular approaches are verified in cells and/or animals, including the use of transgenic (random integration) or knock-in mice, SMC1β^{-/-} mice (already created and described herein), and the establishment of spermatogonial cell cultures that can be triggered to undergo meiosis (Feng et al., Science 97:392-395, 2002).

The newly generated SMC1β^{-/-} strain provides an ideal model for organismal experiments. This strain serves as a proof-of-concept model, because it is clear that homozygous SMC1β^{-/-} mice are viable and live beyond the beginning of the reproductive age (the oldest have now lived over a year). The mice have demonstrated no obvious phenotype beyond a meiotic one. Their testes are smaller and weigh 50% less that of controls. Additionally, the testes are apparently devoid of meiotic cells. The mice have shown to be sterile as demonstrated by breeding experiments. These mice have proven useful not only for the study of SMC1β's role in fertility but also for its role in meiosis. Furthermore, these mice have demonstrated that the elimination of SMC1β expression or activity is an attractive one for inducing infertility without many or any side effects. Likewise, these mice present an interesting model for the study of infertility.

Knock-in mice that carry a specifically mutated SMC1β gene at its natural genomic locus are also being created. Techniques and materials required to generate such transgenic animals are well known in the art and have been used in the generation of the SMC1β^{-/-} mouse (Borggrefe et al., Europ. J. Immunol. 31:2467-2475, 2001). Several modes of regulation of SMC1β may be examined by this approach. Having identified a protease cleavage and/or a phosphorylation site, a point mutation is created in that site by site-directed mutagenesis (Masat et al., Proc Natl Acad Sci USA 97:2180-2184, 2000; Shinohara et al., Nature 416:759-763, 2002). The mutant gene is then inserted in place of the wildtype gene. The mice are then analyzed for their meiotic phenotypes, for chromosomal association of SMC1β, and for SMC1β complex formation. Failure to dissociate SMC1β from the chromosomes, either at metaphase I (chromosome arms) or at metaphase II (centromeres), may arrest meiosis. These experiments also reveal whether the specific proteolysis or phosphorylation event(s) is required for arm or centromer dissociation, or both. Similarly, a site specific for a candidate transcription factor that drives the meiosis-specific expression of SMC1β may be mutated in a knock-in, and the effect of that mutation investigated. If expression of a particular mutant SMC1β gene has a strong dominant negative phenotype, germline transmission may not occur. Transgenic mice with randomly integrated expression constructs are also contemplated. This approach is particularly attractive for testing meiosis- and tissue-specific expression, e.g. of a promoter/enhancer region. A candidate region is linked to the lacZ gene, whose expression can readily be analyzed.

In a different approach, spermatogonial cell culture system (Feng et al., Science 297:392-395, 2002) is utilized. In this system, type A spermatogonial cells are, partially purified from mouse testes using the STA-PUT apparatus. Cells are transfected with an expression vector for mTeRT (telomerase reverse transcriptase), which immortalizes them without transformation. Cultures proliferate and remain undifferentiated for a long time period, but can be induced to undergo meiosis by addition of stem cell factor (SCF; kit ligand). After 1 week of induction, 40% of the cells have been reported to be 4N, and after 3 weeks of induction, 58% of the total cell population are haploid. An established spermatogonial wildtype line is also available from Dr. Martin Dym (Georgetown University). Cultures are also being established from SMC1β^{-/-} spermatogonial cells (SMC1β is not expressed before prophase I of meiosis). This cell system allows for numerous experimental approaches. For example, phosphatase (okadaic acid, vanadate) or kinase inhibitors (Wortmannin, staurosporine, or a variety of others) added to the cells at specific steps of meiosis are analyzed for their effects in culture. Likewise, specific protease inhibitors (as described herein for PEP or separase) are also tested. FACS analysis is used to examine meiotic progression (Borggrefe et al., Europ. J. Immunol. 29:1812-1822, 1999, Borggrefe et al., Europ. J. Immunol. 31:2467-2475, 2001; Stursberg et al., Gene 228:1-12, 1999; and Gross et al., Europ. Immunol. 32:1121-1128, 2002); and immunofluorescence of chromosome spreads. Cell populations, highly enriched for a specific stage of meiosis, are then analyzed for the presence of proteolytic activity. As is common for inhibitor experiments, some conclusions may be limited because some inhibitors are not of high enough specificity (e.g. Wortmannin) or may block a specific enzyme that may be required for other important cellular functions, causing apoptosis or other effects that may perturb analysis. Thus, inhibitor experiments are interpreted in the context of other results. One such approach is the expression of transgenes in spermatogonial cells.

Because cultured cells can be efficiently transfected with expression constructs of ones choice (Feng et al., Science 297:392-395, 2002), wildtype or mutant SMC1β genes are expressed to study their effects on meiosis in wildtype cells. This allows for the expression of mutant kinase or protease genes and the analysis of potential dominant negative phenotypes. Wildtype or mutant SMC1β genes are also being expressed in SMC1β^{-/-} cells to try complementing their phenotype and assess the effect of mutants in this background. Thus, this approach allows for the study of interaction and chromosome association, i.e. structure-function data from this approach. The different roles of SMC1β in meiosis, e.g. in arm cohesion, centromeric cohesion, and perhaps DNA recombination are also being examined in culture. This system allows for the more rapid study of the effects of interfering with SMC1β than the animal models do. Both systems, the cell culture and the animal systems, are complementary, but not dependent on each other.

The expression of small interfering RNA (siRNA) molecules in the spermatogonial cells, as a means to further manipulate SMC1β function, and a precursor of possible future use of SMC1β as a drug target is also contemplated in this invention.

### Example 11

### SMC1β's Role in Meiosis and Fertility

The invention also contemplate the role of SMC1β in vivo, and its relevance for human reproductive health. Interfering with SMC1β provides a novel means to very specifically and effectively block meiosis. SMC1β protein, required for meiotic sister chromatid cohesion and meiotic DNA recombination, has shown to be important for spermatogenesis and male fertility. SMC1β protein also appears to be important for oogenesis as well, because all female SMC1β knockout mice have exhibited infertility. Therefore, SMC1β is a useful protein target for the induction of infertility, for example, as a method of contraception, or the treatment of infertility.

Other meiosis-specific cohesins, Rec8 and STAG3, appear to be less attractive target candidates. Rec8 appears too early, i.e. in the premeiotic division, and STAG3 disappears much earlier than SMC1β, and seems to interact only with a subset of cohesin complexes in mammalian meiosis. In yeast, defective Rec8 transforms the first meiotic division into a mitotic cycle (Watanabe et al., Nature 400:461-464, 2001). The central cohesin SMC1β also proves to be an attractive target, because the cohesin complexes form a protein structure independent of the synaptonemal complex Pelttari et al., Mol. Cell. Biol. 21:5667-5777, 2001). Thus, a multilevel approach that targets the SC proteins and cohesin(s) is envisioned.

In meiosis, chromosomal missegregation is found in frequent chromosomal nondisjunction syndromes including the trisomies 21 (Down syndrome), 18, 16, and 15 (Nicolaidis et al., Human Reprod. 13:313-319, 1998). Correct sister chromatid cohesion and recombination are prerequisites for proper meiotic divisions, and it is plausible to imagine missegregation to happen if, for example, the SMC1β protein, or factors associated with it, were defective. Phenotypic results may be the aneuploidy or sterility syndromes, both with clear medical and social implications. In fact, more than 90% of all cases of trisomy 21 are due to errors in female meiosis, most of them happening in meiosis I. Similar numbers were reported for other trisomies (trisomies 18, 16, 15). Also, about 50% of spontaneous abortions that occur before 15 weeks of gestation are chromosomally aneuploid with a high incidence of trisomies (Nicolaidis et al., Human Reprod. 13:313-319, 1998). Infertility is a very common medical problem that affects about 10% of couples and has a significant familial component, with autosomal recessive inheritance accounting for half of the male cases (Lilford et al., BMJ 309:570-573, 1994). While many genes are probably involved in this complex phenomenon, genes encoding proteins that are key to meiotic chromosome dynamics are among the prime candidates. For example, meiotic arrest at the primary spermatocyte stage may be triggered if sister chromatid cohesion or DNA recombination fail. Such arrest is the most common type of maturation arrest in men with non-obstructive azoospermia (Martin-du Pan et al., Fertil Steril. 60:937-946, 1993). Indeed, analysis of synaptonemal complex formation in patients revealed extensive synaptic anomalies that could be related to the meiotic arrest seen in these individuals (Egozcue et al., Mol. Cell. Endocrinol. 174:310-321, 2000).

While naturally occurring deficiencies in a specific protein like SMC1β likely cause severe fertility problems, artificially disabling SMC1β can be an advantage for regulating reproduction, i.e. for contraception. Conceptually it is attractive to look for a target that is both, highly important and highly specific for meiosis. SMC1β fulfills both requirements. Thus, the function of SMC1β in the organism is studied and interfered with to understand it's role in the induction of infertility (as a contraceptive) and in the treatment of infertility. The regulation of SMC1β expression, activity, and function provide targets for contraceptive intervention, like meiosis-specific transcription factors or components of specific proteolysis pathways.

### Example 12

### SMC1β Toxicity in Somatic Cells

Experimental evidence has suggested that SMC1β may be toxic if expressed in somatic cells. Expression constructs, that allow for inducible expression, for either the somatic SMC1α or the meiotic SMC1β were transfected into 293 cells (a somatic cell line), and stable clones were selected on the basis of neomycin-resistance. While more than 10 clones that stably (long-term) expressed SMC1α were obtained, no clones were obtained for SMC1β. Two clones that expressed SMC1β initially stopped expressing it after a few passages. After the induction of expression, cells appeared to die.

These experiments are being repeated. Additional experiments include the use of transient gene expression, and the resulting phenotypes are being examined for apoptosis and cell cycle arrest. However, if SMC1β is toxic or detrimental to somatic cells, it provides the basis for an assay with which to screen compounds or agents that inhibit SMC1β expression or activity.

### Example 13

### SMC1β is Required for Meiosis, Sister Chromatid Cohesion and DNA Recombination

Sister chromatid cohesion ensures faithful segregation of chromosomes in mitosis and meiosis. For meiosis, cohesion has been specifically adapted to facilitate both meiotic divisions. To determine if SMC1β, a meiosis-specific component of the cohesin complex, was responsible for most sister chromatid cohesion, including centromeric cohesion, SMC1β knockout mice were analyzed.

SMC1β-deficient mice of both genders were found sterile. Male meiosis was found blocked in the mid-pachytene stage, while female meiosis was highly error-prone but continued until metaphase II. Prophase axial elements were dramatically shortened and chromatin loops enlarged; chromosome synapsis was incomplete; and sister chromatid cohesion in chromosome arms and at centromeres was prematurely lost. In addition, crossover-associated recombination foci were absent or reduced, and meiosis-specific perinuclear telomere arrangements were impaired. Thus, SMC1β is important for meiotic cohesion, synapsis, recombination, and chromosome movements.

Consequently, analysis of the first cohesin-deficient mouse mutant showed that meiosis-specific SMC1β is important for key processes in male and female meiosis. SMC1β is needed from the onset of meiosis for orderly chromosome processing, including telomere clustering and axial element assembly, as it is for sister chromatid cohesion and DNA recombination. Experimental findings also illustrate the intimate links between meiotic cohesion, synapsis, and recombination. It is conceivable that malfunctioning of SMC1β may be one cause of infertility and for the frequent meiotic chromosomal missegregation in humans. Specific details of how the loss of SMC1β in SMC1β−/− mice affected spermatogenesis and oogenesis are set out below.

Spermatogenesis

Testes of 5-week-old SMC1β−/− mice were only half the size and weight of those of heterozygous or wildtype littermates. This difference increased with age as the wildtype testes grew larger. In testicular seminiferous tubules, Sertoli cells and spermatogonia were present in comparable numbers as in wildtype or heterozygous mice. However, stages beyond pachytene were completely absent, and the tubules did not contain spermatids. From analysis of the testicular cell associations in SMC1β−/− mice, it was inferred that spermatocytes developed no further than mid-pachytene. Using the TUNEL assay, apoptosis was analyzed to determine whether this process might be responsible for the lack of development beyond the pachytene stage. On days 15-19 post partum, when the first synchronized wave of meiotic cells reaches the pachytene stage, increased numbers of apoptotic cells were found at day 17. Immunostaining for the pachytene-specific histone variant H1t16 showed that only 1% of spermatocytes in adult mice reached the mid-pachytene stage (24 of 2014 analyzed nuclei). Thus, SMC1β−/− spermatocytes die by apoptosis within a brief temporal window upon reaching the pachytene stage.

Meiotic chromosome structures in wildtype or mutant spermatocytes were also analyzed by staining chromosome spreads with antibodies specific for components of the synaptonemal complex (SC), such as SYCP1 or SYCP3. SCs in SMC1β−/− spermatocytes were dramatically shortened by about 50%. This phenotype has not been observed previously with any mammalian meiotic mutant. Chromosome synapsis in mutant meiocytes is rarely complete, but the number of AEs was normal at 40 (19 bivalents/SCs and the sex chromosomes). The shortened SMC1β−/− SCs did not represent fragmented chromosomes, since they all bear centromeres and telomeres exist at both ends of most chromosomes. Each individual chromosome was shortened, and the extent of shortening was the same for each chromosome, since the length of axial elements (AEs) relative to each other within each genotype was unchanged. If cohesin not only maintains cohesion between sister chromatids, but also tethers the chromosome loops to the SC, the lack of the major meiosis-specific cohesin may significantly reduce the number of sites at which chromosome loops form their base. The AE of the SC would then contain less DNA and therefore be shorter, while the chromosome loops should be extended. Non-SC chromatin was assessed by chromosome painting using probes that labeled chromosomes 1 and 13 in surface spread spermatocytes. Measurement of the distance between the SCP3-labeled axes and the most distal margin of the paint signal, i.e. the outer edge of the chromatin loops, revealed an almost two-fold increase in chromatin loop extension (SMC1β+/+: 2.60+/−0.63 μm; n=19. SMC1β−/−: 4.79+/−1.2 μm; n=19; T-test: p<0.001), which suggests that SMC1β cohesin determines DNA loop attachment along the AE and thus chromatin packaging into the SC.

Some of the SMC1β/SMC3-type cohesin is present in early prophase cells until it disappears in late prophase I. This cohesin may allow the attachment of limited numbers of chromosome loops to the AB, resulting in the shortened AEs in SMC1β−/− spermatocytes. To evaluate the effect of SMC1β deficiency on the presence and localization of other components of meiotic cohesin, a series of immunostainings for REC8, STAG3, and SMC3 were performed. All three components were present on SMC1β−/− zygotene or early pachytene chromosomes; but their staining pattern differed from that in wildtype cells. SMC3 and REC8 were reported to uniformly stain all along the chromosomal axes. On SMC1β−/− chromosomes, SMC3 and REC8 appeared in a dot-like pattern. This pattern is similar to that reported for SMC1β and thus may reflect the SMC1β-type prophase complex in these cells. Analyzing that complex by immunoprecipitation of nuclear extracts from testes of wildtype or mutant mice demonstrated association of SMC1β with REC8 and STAG3 and thus formation of a meiosis-specific complex, which in early meiosis may exist alongside the mitotic SMC1β complex.

In meiotic prophase, SMC1β protein has been observed at bridges between the AEs of homologs, supposedly sites of future chiasmata. Early markers for crossovers are the MLH1 and MLH3 mismatch repair proteins, which in wildtype form foci at such sites. Foci formation is completely absent in SMC1β−/− spermatocytes. With exception of the shortest mouse chromosomes, AE length has generally been correlated to the number of MLH1 foci, and the reduced AE length in the mutant fits that model. Earlier steps in meiotic recombination are marked by other proteins, including RAD5120. Normal RAD51 foci formation in leptotene and zygotene cells suggested that SMC1β−/− spermatocytes are capable of initiating recombination. Similarly, the assembly of the histone variant γ-H2AX, a marker for double-strand breaks and unsynapsed regions, or the localization of ATR, which marks unsynapsed regions at the zygotene-pachytene transition, looked similar in wildtype and SMC1β−/− spermatocytes. However, at regions of incomplete synapsis, which are more frequent in mutant cells, γ-H2AX, RAD51, and ATR persist. Thus, meiotic recombination is initiated, but crossover does not form in SMC1β−/− spermatocytes.

To determine chromosome dynamics at the entry of meiosis in SMC1β−/− spermatocytes, centromere movements and telomere clustering at the transition from preleptotene into leptotene by FISH with probes specific for pericentric satellite DNA or telomeric sequences were assessed. In wildtype spermatocytes, all telomeres and associated pericentromeres positioned on the nuclear periphery from late leptotene to early diplotene, and formed abouquet at the leptotene/zygotene transition. In SMC1β−/− spermatocytes, however, 2 to 7 telomeres (mean 4.2±1.4; n=29) remained in the interior of the nuclei. The number of pericentric satellite clusters in SMC1β−/− spermatocytes was also significantly increased compared to wildtype (12.2±2 vs 7.6±1.3; p<0.001, T-test). The SMC1β-deficient mouse represents the first mammalian mutant showing defects in meiotic telomere attachment.

To evaluate whether cohesion is defective in SMC1β−/− metaphase I cells, cultured spermatocytes were treated with the phosphatase inhibitor okadaic acid (OA) to induce premature transition into metaphase I. In wildtype mice, OA caused premature chromosome condensation from mid-pachytene on, without affecting sister chromatid arm and centromere cohesion. As a result, 20 bivalents, each with two pairs of unseparated, closely apposed sister kinetochores and at least one chiasma became visible. In contrast, many SMC1β−/− spermatocytes showed 80 separate centromere signals from completely separated chromatids upon OA treatment, indicating a role for SMC1β in centromeric cohesion. In these cells, REC8 was still associated with the prematurely condensed chromatids, but was notably absent from the centromeres. Some cells showed 40 centromeres and thus maintained centromeric sister chromatid cohesion on separated homologs, which correlated with the presence of REC8. Co-staining for γ-H2AX, which decreases from zygotene to pachytene, allowed for determination of the stage at which the cells were exposed to OA. Cells with 80 centromere signals were of pachytene origin (no γ-H2AX), and cells with 40 centromere signals were of zygotene origin (patchy γ-H2AX). Thus, pachytene SMC1β−/− cells that attempted to condense chromosomes preferentially showed centromeric loss, indicating that initiation of chromosome condensation contributes to the loss of centromeric cohesion if SMC1β is defective. However, chromosome condensation did not succeed in SMC1β−/− cells, although the condesin component, SMC4, correctly localized to the mutant chromosomes.

Oogenesis

Female meiosis is known to be more error-prone and apparently less strictly controlled. As a result, the phenotype of meiotic mutants is often milder in the female than in the male. This was also seen for the SMC1β mutation, where a proportion of oocytes were able to proceed beyond the pachytene stage and enter dictyate arrest. As in the male, SCs in pachytene stage oocytes from mutant females were shorter. In addition, a variety of synaptic defects were noted, including an increase in SCs with gaps and in cells with more than 20 axial elements, indicating either defects in synapsis or premature desynapsis. Among pachytene cells with apparently normal synapsis, the number of MLH1 foci were significantly reduced. However, the distribution of foci appeared normal, with the decrease reflecting both an increase in SCs lacking a focus (E0) and a decrease in SCs with two or three foci (E2 or E3). Female AEs are generally longer than male AEs. Thus, the SMC1β−/− mouse highlights the correlation between AE length and chiasmata: very short male AEs have no chiasmata, while longer female AEs have some chiasmata. Gross disturbances in synapsis would be expected to increase the frequency of germ cells lost via apoptosis, reducing the pool of oocytes. This expectation was supported by the observation that ovary size in mature mutant females was reduced. With increasing age, follicles became rare in SMC1β−/− mice, indicating a depletion of the oocyte pool and a shortened reproductive life. Unlike the SMC1β−/− male, however, diplotene cells and cells entering dictyate arrest were common in the perinatal ovary. However, mutant diplotene cells were morphologically abnormal.

To assess the effect of the lack of SMC1β during the meiotic divisions, oocytes were analyzed at various times during the resumption and completion of the first meiotic division. A cohort of follicles initiated growth in the immature female, and a large number of growing follicles were present in the ovaries of mutant females at this stage. Ooctyes collected from mature follicles, and meiotically matured in vitro, exhibited normal rates of germinal vesicle breakdown, i.e. meiotic resumption, and first polar body extrusion. However, chromosome preparations from these oocytes exhibited a variety of defects. Consistent with the reduction in MLH1 foci at the pachytene stage, a reduction in the number of chiasmata and an increase in the frequency of univalent chromosomes were observed at diakinesis/metaphase I. Further, in most metaphase I cells, loss of cohesion in both the chromosome arms and at the centromeres was evident. Interestingly, as cells progressed to metaphase I, the frequency of univalents increased. Univalents were observed in 19% of cells collected immediately after nuclear envelope breakdown and in more than 60% of cells collected at metaphase I. Finally, among oocytes that had extruded a first polar body and arrested normally at second meiotic metaphase, virtually no typical metaphase II chromosome complements were observed. Instead, the vast majority of chromosomes were present as unpaired single chromatids, indicating premature loss of cohesion at sister centromeres. Exemplary methods for performing these experiments are set out below.

Generation of SMC1β−/− Mice

The mouse gene for SMC1β is located on chromosome 15 (Ensemble gene ID ENSMUSG00000022432; mouse SMC1β mRNA, GenBank accession number NM_{—}080470 (SEQ ID NO: 1); mouse SMC1β protein, GenBank accession number NP_{—}536718 (SEQ ID NO: 2)]). The human homolog was found in a syntenic region on human chromosome 22 (human SMC1β; GenBank accession number NT_{—}011522), and is known as human SMC1β, GeneID 27127 [human SMC1β mRNA, GenBank accession number NM_{—}148674 (SEQ ID NO: 3); human SMC1β protein, GenBank accession number NP_{—}683515 (SEQ ID NO: 4)]. The coding sequence position in chromosome 22, contig NC_{—}000022, is from 44060974 to 44129985. In both genomes, the locus contains another gene located in close proximity to the SMC1β gene. In the human genome, the nucleotide sequence for the second gene which lies in close proximity, GeneID 26150, is GenBank accession number NM_{—}015653 (SEQ ID NO: 5) which encodes the amino acid sequence, GenBank accession number NP_{—}056468 (SEQ ID NO: 6). In the mouse genome, the nucleotide sequence for the second gene which lies in close proximity is GenBank accession number AK01631 1 (SEQ ID NO: 7), which encodes the protein, GenBank accession number BAB30190 (SEQ ID NO: 8; also identified as GenBank accession number AAH09904). The two genes are arranged in a head-to-head configuration. In the mouse genome, the distance between the 5′ end of the SMC1β cDNA and the 5′ end of the AK016311 cDNA is only 142 bp. In the human genoine, the start of a cDNA clone and SMC1β start codon are 162 bp apart. Therefore, it is very likely that the 5′ region of the SMC1β gene contains a bidirectional promoter, or two overlapping promoters.

In the mouse, the second gene is transcribed in testes, and probably transcription of these two genes is coordinated and controlled by overlapping promoter elements. To avoid possible interference with expression of the second gene, a vector was designed for targeting of exon 10, located 19 kb apart from the start codon. Exon 10 codes for 40% of the hinge domain which is important for the SMC heterodimer. The linearized targeting vector was electroporated into W4/129S6 embryonic stem (ES) cells (Taconic Inc.). Production of targeted ES clones was performed according to standard procedures. Homologous recombinant ES cell clones, identified by Southern blotting using 5′ and 3′ probes, were injected into C57B1/6 blastocysts. The resulting chimeras crossed with C57BL/6 mice transmitted the disrupted allele through the germline.

Histological Analysis and Terminal Deoxynucleotidil Transferase-Mediated dUTP Nick End Labeling (TUNEL) Assay

Testes and ovaries were fixed in Bouin's solution (Sigma) for 18 h at 4° C., washed with 70% ethanol for 24 h and embedded in paraffin. Testis sections were stained with periodic acid, Schiff's solution, and hematoxylin. Ovary sections were stained with hematoxylin and eosin. For the TUNEL assay, testes were fixed with 4% formaldehyde in phosphate buffer for 16 h at 4° C. and embedded in paraffin. TUNEL assay on sections was performed using the TACSTM 2 TdT-DAB kit (Trevigen Inc.).

Immunoblot Analysis and Immunoprecipitation

Nuclear extracts were prepared and immunoprecipitations were performed as described by Revenkova et al. (Mol. Cel. Biol. 21:6984-6998, 2001) using rabbit polyclonal anti-SMC3 antibody (Eijpe et al. J. Cell Sci. 113:673-682, 2000) and agarose-immobilized anti-SMC1 antibody (Bethyl Inc.). For immunoblot analysis we used the following antibodies: anti-SMC1α (Eijpe et al., supra, 2000), anti-SMC3 (Eijpe et al., supra, 2000), anti-SMC1β (Revenkova et al., supra, 2001), and rabbit polyclonal anti-STAG3, raised against a recombinant polypeptide representing amino acids 640-773 of mouse STAG3, and a mouse mAb against RNA Pol II (mAb clone 8WG16) from Covane Research Products.

Immunocytochemistry

Most antibodies used have been described (Eijpe et al., supra, 2000; Revenkova et al., supra, 2001; and 14. Eijpe et al., J. Cell Biol. 3,.657-670, 2003). Mouse anti-MLH1 was from BDPharMingen, rabbit-antiMLH1 from Calbiochem, and rabbit anti-SMC1α from Bethyl Labs Inc. Mouse anti-γH2AX was donated by A. Pastink, rabbit anti-SMC3 by J. -M. Peters, rabbit anti-RAP1 by T. de Lange, mouse anti-ATR by P. de Boer, mouse anti-MLH3 by P. Cohen, and goat anti-rat SCP3 by T. Ashley. Paraffin and frozen sections of mouse testis, and dry-down preparations of testis cell suspensions were prepared, incubated for immunocytochemistry and analyzed as described (Eijpe et al., supra, 2000;, Revenkova et al., supra, 2001; Eijpe et al., J. Cell Biol. 3, 657-670, 2003; Peters et al., Chromosome Res. 5:66-68, 1997). Mice were labeled in vivo with BrdU, and detected -the incorporated BrdU in sections and dry-down preparations as described before (Eijpe et al, supra, 2003). In some experiments, testis cell suspensions were exposed to 5 μM okadaic acid for 5 h as described (Wiltshire et al., Dev. Biol. 169, 57-67, 1995) before spreading the cells by the dry-down procedure (Peters et al., supra, 1997).

For all studies of MI and MII female chromosomes, prophase arrested oocytes were obtained from 3.5-4-wk-old females. Ovaries were removed and placed in Waymouth's MB752/1 medium supplemented with 10% fetal calf serum and 0.23 mM sodium pyruvate. Antral follicles were punctured with 26 gauge needles to obtain oocytes at the germinal vesicle stage which were subsequently cultured in microdrops of medium under oil at 37° C. in 5% CO₂ in air. Oocytes, in which germinal vesicle breakdown did not occur within 2 hours, were excluded from these studies due to failure to resume MI. Air-dried chromosome preparations were made from the remaining oocytes using the technique described by Tarkowski (Cytogenetics 5:394-400,, 1966). Preparations were stained with 400 ng/ml of 4′,6-diamidino-2-phenylindole (DAPI) and viewed using a Zeiss Axiophot epifluorescence microscope.

For immunostaining surface spread synaptonemal complex preparations were prepared from ovaries of newborn female mice as described previously (Peters et al., supra, 1997) and incubated as described by Koehler et al. (Genetics 162: 297-306, 2002). Primary antibodies used were a goat antibody raised against rat SCP3 and rabbit anti-MLH1. Secondary antibodies used were fluorescein-labeled donkey anti-rabbit and rhodamine-labeled donkey anti-goat (Jackson Immunoresearch, PA). Cells were viewed using a Zeiss Axiophot epifluorescence microscope.

Analysis of Bouquet Formation in Spermatocytes

Testes suspensions yielding structurally preserved nuclei for simultaneous SC immunostaining, FISH, and bouquet analysis were prepared and analyzed as described previously by Scherthan et al. (Mol. Biol. Cell 11:4189-4203, 2000). Preleptotene nuclei were identified by major satellite DNA lining the nuclear periphery, while most telromeres were interior; bouquet nuclei were identified by most telomeres accumulated at a limited sector of the nuclear periphery (Scherthan et al., J. Cell Biol. 134:1109-1125, 1996). Preparations were evaluated using a Zeiss Axioskop epifluorescence microscope (Carl Zeiss) equipped with single and double-band pass filters for excitation of blue, red and green fluorescence (Chroma Technologies). Images were recorded with a cooled CCD camera (Hamamatsu Photonics) using the ISIS fluorescence image analysis system (MetaSystems). Images were further processed using Adobe Photoshop to match the fluorescence intensity seen in the microscope. Three-dimensional evaluation of immunostained nuclei was performed in some experiments using a Zeiss Axioskop epifluorescence microscope equipped with a 100× plan-neofluar oil-immersion lens (NA 1.35, ZEISS) attached to a PIFOC z-SCAN (Physik Instrumente, Germany), and a 12 bit CCD digital camera (PCO, SensiCam, Germany) controlled by TILLvisION v4.0 software. Fluorochromes were excited using a polychrome IV monochromator (TILL Photonics, Germany) in combination with a quadruple band pass beam splitter and barrier filter (Chroma). Images spaced 0.2 μm were used to obtain 3D movies using the Surpass module of Imaris3.3.2 (Bitplane, Switzerland).

### Example 14

### Identification of the Putative SMC1β Promoter/Enhancer

To identify the SMC1β promoter/enhancer and determine it's germ cell-specificity, the putative promotor/enhancer region of mouse SMC1β, was placed in a vector upstream of enhanced green fluorescent protein and put it into mice. Details of this procedure are described in Example 4 as set out above. Specifically, human cytomegalovirus promoter (pCMV) was removed from vector pEGFP-C1 [BD Biosciences Clontech, GenBank Accession No. U55763 (SEQ ID NO: 9)] by digestion with restriction endonucleases AseI and AgeI. SMC1β promoter sequence was amplified by PCR using the primers 5′-TTT TAT TAA TCA CGG CAA GAA AAG CCC AC-3′ (SEQ ID NO: 10) and 5′-TTT TAC CGG TGC CTC AGC CGC TTC C-3′ (SEQ ID NO: 11). The mouse SMC1β promoter region (SEQ ID NO: 12) is set out below:

AGAAAAGCCC ACAGAAATAA GCAAAGCTAC AGCGCCGAGA
AGCAGCGCCC ACAAAAGTTT GAGAGCAAGT CTGTCCCGGC
GGTCCGCTTT CCGCAGCGGA GCAGGGATCC AATTCCCCGG
GCAACGCCAA CCTCCGTTGT AAGCAACGGC GCCTCGCTCG
CTCTCCTTCC CCCGCGCCAG TCTCGCGAGA CTTCGAAAAG
AATTTCTTCC CGCGCTTTTT TTTTTTTTTT TCCTCACGGG
AGCACGAGGA AGCGGCTGAG GCACGGCGCG CAGCCATG

The SMC1β start codon is underlined. The sequence shown in bold was amplified by PCR and tested for promoter activity. The amplified fragment was digested with restriction endonucleases AseI and AgeI and to AseI/AgeI—digested pEGFP-C1 upstream of enhanced green fluorescent protein (EGFP) gene. The resulting construct was named pBetaSEGFP. To obtain transgenic mice, the DrdI fragment of pBetaSEGFP, which contains the SMC1β promoter region, EGFP gene, and SV40 early mRNA polyadenylation signal, was used for microinjection. For generation of transgenic mice, the expression constructs were injected by the Mt. Sinai Mouse Genetics Shared Facility, and five founders with different insertion sites and different numbers of transgene copies were selected. Verification of transgenic mice was performed by Southern analysis of tail DNA. Cell suspensions were prepared from testes of transgenic mice derived from different founders and green fluorescent cells were counted by FACS. In mouse #1, 84% of testicular cells expressed GFP; in mouse #2, 44% of testicular cells expressed GFP; and in mouse #3, 8% of testicular cells expressed GFP. Thus, the SMC1β promoter fragment is active in testes. To examine GFP expression at different stages of germ cell differentiation, testes are fixed in formaldehyde, embedded in paraffin, and sections are stained for immunohistochemical expression of GFP.

The mouse promoter sequence, which was used in the EGFP construct and which is active in testis, has 58.2% identity with the corresponding human SMC1β promoter region sequence (SEQ ID NO: 13) as set out below:

AGAACAGGCG ACGAAAATAA GGAAGCTGTA GCGCAGGCAA
GGTCGGCTCC TTCGGTCGGG CAGGGGAAAG ACAAGCCTTT
AGCGCAGTTT TCCGTCGGGC TCCCAGTGAC GCCGCAGAGG
TACAGACGCT CCCGCTCTCT CGGAAATGTC AACAACTCGT
TGCTAAGGAA CGGCTCCGCG CTTGCCGCGT CTCGCTCTCT
TCTCGCGACA CTTGGCGAAT CCCTTCCCGC GCTTTTTCCG
CGGGCGCTTG ATAACGCGGG TGAGGCG

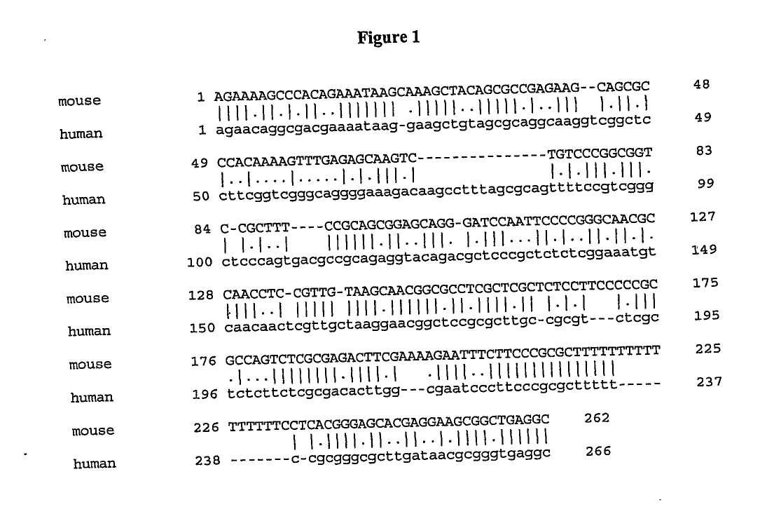

An alignment of the mouse and human SMC1β promoter sequences (SEQ ID NOS: 12 and 13) is provided in FIG. 1. The identification of these putative promotor/enhancer regions of SMC1β provides a useful tool for driving germ cell-specific expression of any gene of interest.

It should be understood that the foregoing description relates to preferred embodiments of the invention and equivalents and variations that will be apparent to the reader are also intended as aspects of the invention. The references referred to herein throughout are incorporated by reference.

1. A method for inducing infertility in an animal comprising inhibiting SMC1β expression or activity in said animal.

2. The method of claim 1, wherein said inhibiting comprises contacting said animal with a nucleic acid selected from the group consisting of a nucleic acid that is an antisense SMC1β nucleic acid and a compound 8 to 80 nucleotides in length targeted to a nucleic acid molecule encoding SMC1β, wherein said compound specifically hybridizes with a nucleic acid molecule of SEQ ID NO: 1 or 3 and inhibits the expression of SMC1β.

3-9. (canceled)

10. A method for inducing infertility in an animal, comprising administering to an animal an effective contraceptive amount, of an agent that inhibits SMC1β expression or activity.

11. The method of claim 10 which further comprises restoring fertility to said animal by ceasing administration of said agent.

12. The method of claim 10, wherein said infertility is caused by blocking spermatogenesis.

13. The method of claim 12, wherein said spermatogenesis is blocked by inhibiting meiosis.

14. The method of claim 10, wherein said infertility is caused by blocking oogenesis.

15. The method of claim 14, wherein said oogenesis is blocked by inhibiting meiosis.

16. The method of claims 13 or 15, wherein said meiosis is inhibited at prophase of meiosis I or later.

17. The method of claim 10, wherein said agent is selected from the group consisting of: a nucleic acid construct, a small molecule antagonist of SMC1β, a peptidomimetic antagonist of SMC1β, and an anti-SMC1β antibody.

18. The method of claim 17, wherein the agent is administered in a composition comprising a pharmaceutically acceptable carrier.

19. The composition of claim 18, wherein the pharmaceutically acceptable carrier is an adjuvant, solubilizer, stabilizer, diluent, anti-oxidant, liposome, micelle, or patch.

20-28. (canceled)

29. The method of claim 18, wherein the agent is administered orally, parenterally, topically, transdermally, systemically, intravenously, intraarterially, intraperitoneally, or intramuscularly.

30. The method of claim 12, wherein the administration is to the testis.

31. The method of claim 30, wherein the administration to the testis is by a route selected from the group consisting of: injection, implantation, and transdermal application.

32. The method of claim 14, wherein the administration is to the ovary.

33. (canceled)

34. The method of claim 10, wherein the animal is human.

35. A method of inhibiting meiosis in germ cells, comprising inhibiting the expression or activity of SMC1β in said cells.

36. The method of claim 35, wherein said germ cells are spermatocytes.

37. The method of claim 35, wherein said germ cells are oocytes.

38. The method of claim 35, wherein said meiosis is inhibited at prophase of meiosis I.

39. The method of claim 38, wherein said cells are treated in vitro.

40. The method of claim 38, wherein said cells are treated in vivo.

41. The method of claim 38, wherein said cells are treated in an animal subject.

42. The method of claim 41, wherein said subject is human.

43. The method of claim 35, wherein said method comprises contacting said cells with an agent that reduces the expression or activity of SMC1β.

44. The method of claim 43, wherein said agent is a nucleic acid construct.

45-47. (canceled)

48. The method of claim 43 or 44, wherein the agent is administered in a composition comprising a pharmaceutically acceptable carrier.

49. The method of claim 48, wherein the pharmaceutically acceptable carrier is an adjuvant, solubilizer, stabilizer, diluent, anti-oxidant, liposome, micelle, or patch.

50-133. (canceled)

134. A method according to claim 1 or 10 substantially as described and illustrated herein.

135-138. (canceled)


SEQUENCE LISTING (13 sequences)

SEQ ID NO: 1 - DNA, 4056 bp, Mus musculus
ctgaggcacggcgcgcagccatggggcaccttgagctgctgctcgtggagaatttcaagtcgtggcgaggccgccaggtcatcggccctttcaagaggttcacctgcatcattggccccaacggctccggaaaatctaatgtaatggatgcacttagttttgtaatgggagaaaagacaactaatttaagagtaaaaaacattcaagaacttattcatggagcacatactggaaaacctgtttcttcttctgcaagtgtgacaattatatacatagaggacagtggagaagagaaaacattcacaaggattatccgaggggggtgctcagaatatcattttggggataaacccgtgagtcgttctgtgtatgtagcccagttggaaaacataggcataatagtcaaagcacagaactgtctagtttttcagggaactgtagagtcaatatccatgaaaaagcccaaagagagaacccagttttttgaagaaatcagtacttcaggagaatttataggagaatatgaggcaaagaaaaagaaattacaaaaagctgaagaagatgcacaatttcattttaatgtgaaaaaaaatgtagctgcagaacggaagcatgcaaaaatagagaaagaagaggcagaacattaccaaaatctccttgaagaattgaaaataaacaagatacaactgatgcttttccaactatattataatgaggaaaaaatcaatgttttaaacactgaattagagcaaatggacgggaatttaagtgttgtaaaagacactctttctcaccatgaaaacatatttaaagctaagaaaaaggattatggaatgttaactagacaactacagcaaacagcaaaagaactgaaatctgttgaagcaattttaaatcagaagaggcctcagtacattaaggctaaagaaaacacttctcatcatctaaaaaaattagatttgtctaagaaattgataacagacaatgaaaaacaatgttctaagcaggaagatggcatacgagccttagtggcagaactggctgatttggatagagcatggaaaagttttgaaaagcagatggaagagaaaatcttacaaaaagggcgagatattgaattggaaaatagccagctggatcgttacaaactgcttaaggagcaagtcagacggaaggttggtataatgacacaacaactggaaaaactgcagtgggaacagaaggcagaaaaagaaagacttgcatttgaaaagaggaggcatggagacactcagggaaatctaaaacagataaaagaacaaatagaagagcataaaaaacgaatagagaagttggaggaatatacaaagacgtgcatggattgcttggaagataaaaaacagcaagaagaggccctgaaaaaagaaattgaaaatacgaaatcaagaatgtctgaagttaatgaagaattgagtcttattagaaatgaattgcagaatgctggaattgataaccatgagggaaaacgtcagcagaaaagagcagaagttctggaacaccttaaaagactttacccagattctgtgtttggaagactgcttgatctgtgtcatcctattcataagaagtaccagctggctgtgactaagctttttggccggtacatggttgccattgttgtagcctcagaaaagatagcgaaagattgtattcgatttctgaaggcagaaagagctgaacctgagacattccttgctctagattaccttgatatcaagccaatcaatgaacgactaagggaaattaaaggctgtaagatgatgatcgatgttataaagacccagtttcctcagctgaagaaagtgattcagtttgtttgtggaaatggccttgtctgtgagactgtggaagaagcaagacatattgcattcggtggacctgaaagacggaaggcagtagcacttgatggaacactgtttttgaaatctggagtgatttctggagggtcaagtgacttaaagcacaaagctctgtgctgggatgagaaagagttacacaatctaagagacaaaagaagccaactagtccaagagctaaaggagttaatgaagacactccgcaaggaaacagatctgaagcaaatacagactttagtacaaggaaccaatacacgactcaaatattcacaaaatgaactagagatgattaaaaagaagcaccttgctacattttaccgggaacaatctcagctacaaagtgaattactgaatattgattctcaatgtactatgttgagtgaaggaatcaacaaacagcaacaaaaaattgaagaatttcaagataagatagatgaggtagaagatgacatattccaagacttctgtgaagaaattggtgtggaaaatatccgtgaatttgagaataaacatgttaaacagcagcaagaaaatgatcaaaaaagattagagtttgaaaaacaaaaaactcggcttaatattcagctcgaatatagtcgaaatcagcttaagaagaaactgaataatatcgacacattaaaaaccacaatccagaagggcaaagaagatattgataacctaaaaaagaccgaagaagaatgtctgaaaattgttgaggaactcatggtgaagcaagagcaaattaaggaagtgcttgccacacagagttccaacattgaaaaaattcacatacaaattgaagaggaacgcaagaaggttttggctgttgatagggaagttggaaaattacagaaggaagttgtaatcattcagggttctttggaacagaaactgctagagaaacataacttgctgctagattgcaaagttcaagacattgacataagtcttgtgctggggtcattggaggacatcattgaaatggagctaactgaaacagaaagcacccaggcaacagctgatatctatgagaaagaagcatccatccaaatagactacagccctctaagggaggatttaaaggctctacaatcagataaggaggtggaggcccaccttacactccttctacagcaagtagcatcccaagaaaacactctactgaagactacagctccaaacttgcgagcacaggagaacttaaagactgtcagagacaagtttcaagaatctgcagatgtttttgaggccagcagaaaggaagccagaatatgtaggcaagagtttgaacaggtgaaaagacggaggtacgatgctttcagtcaatgttttgaacacatctcagtctcaattgatcaaatctacaagaagctctgcaggaacaacagtgcccaggcatttcttagcccagagaaccctgaagaaccttacttagatggaattagctacaactgtgtggctccaggcaaacggttcatgcccatggacaacctgtcagggggagaaaagtgtgtggctgctctggctcttctgtttgctgtacacagttttcggcctgctccattctttgtattagatgaagtagatgcagccctggacaatactaacattggcaaagtctcaagttacatcaaagagcagagtcaggaacagtttcagatgataatcatttccctgaaagaggagttctactccaaagctgatgcactgataggcgtctacccagagcacaatgagtgcatgttcagccatgtgttgactctggacctttccaagtatccagacaccgaagaccaagaaggcagcaggagccaccggaagcccagagtaccacgagtatcaatgtctccaaagtctccccagtctcgttagaaatgagtatactcttgaacagccaccagctatagaagctttaccctgaccctgtggaggtcaggccagtctctgtttagacccatgtgtcatagaagaaaaatctcttaccattttagtagaaaaaaaactatagcccactcagctaaggagtgctcagtgtcattccaatggaggttcctatggcatcaatgaatgaggtatatggttttcttttgcttttaagggcttttgttttgaaacaaattggttataaaattttgaattctgcaaaaaaaaaaaaaaaaaa

SEQ ID NO: 2 - protein, 1248 aa, Mus musculus
MGHLELLLVENFKSWRGRQVIGPFKRFTCIIGPNGSGKSNVMDALSFVMGEKTTNLRVKNIQELIHGAHTGKPVSSSASVTIIYIEDSGEEKTFTRIIRGGCSEYHFGDKPVSRSVYVAQLENIGIIVKAQNCLVFQGTVESISMKKPKERTQFFEEISTSGEFIGEYEAKKKKLQKAEEDAQFHFNVKKNVAAERKHAKIEKEEAEHYQNLLEELKINKIQLMLFQLYYNEEKINVLNTELEQMDGNLSVVKDTLSHHENIFKAKKKDYGMLTRQLQQTAKELKSVEAILNQKRPQYIKAKENTSHHLKKLDLSKKLITDNEKQCSKQEDGIRALVAELADLDRAWKSFEKQMEEKILQKGRDIELENSQLDRYKLLKEQVRRKVGIMTQQLEKLQWEQKAEKERLAFEKRRHGDTQGNLKQIKEQIEEHKKRIEKLEEYTKTCMDCLEDKKQQEEALKKEIENTKSRMSEVNEELSLIRNELQNAGIDNHEGKRQQKRAEVLEHLKRLYPDSVFGRLLDLCHPIHKKYQLAVTKLFGRYMVAIVVASEKIAKDCIRFLKAERAEPETFLALDYLDIKPINERLREIKGCKMMIDVIKTQFPQLKKVIQFVCGNGLVCETVEEARHIAFGGPERRKAVALDGTLFLKSGVISGGSSDLKHKALCWDEKELHNLRDKRSQLVQELKELMKTLRKETDLKQIQTLVQGTNTRLKYSQNELEMIKKKHLATFYREQSQLQSELLNIDSQCTMLSEGINKQQQKIEEFQDKIDEVEDDIFQDFCEEIGVENIREFENKHVKQQQENDQKRLEFEKQKTRLNIQLEYSRNQLKKKLNNIDTLKTTIQKGKEDIDNLKKTEEECLKIVEELMVKQEQIKEVLATQSSNIEKIHIQIEEERKKVLAVDREVGKLQKEVVIIQGSLEQKLLEKHNLLLDCKVQDIDISLVLGSLEDIIEMELTETESTQATADIYEKEASIQIDYSPLREDLKALQSDKEVEAHLTLLLQQVASQENTLLKTTAPNLRAQENLKTVRDKFQESADVFEASRKEARICRQEFEQVKRRRYDAFSQCFEHISVSIDQIYKKLCRNNSAQAFLSPENPEEPYLDGISYNCVAPGKRFMPMDNLSGGEKCVAALALLFAVHSFRPAPFFVLDEVDAALDNTNIGKVSSYIKEQSQEQFQMIIISLKEEFYSKADALIGVYPEHNECMFSHVLTLDLSKYPDTEDQEGSRSHRKPRVPRVSMSPKSPQSR

SEQ ID NO: 3 - DNA, 3839 bp, Homo sapiens
cgctttttccgcgggcgcttgataacgcgggtgaggcgtggagggcggcgccatggcccacctggagctgctgcttgtggaaaatttcaagtcgtggcggggccgccaggtcattggccccttccggaggttcacctgcatcatcggccccaacggctctggaaaatctaatgtaatggatgcacttagttttgtaatgggagagaaaatagctaatttaagagtgaaaaatattcaagaactcattcatggagcacatattggaaaacctatttcttcttctgcaagtgtaaaaattatatatgtggaggaaagtggcgaagagaaaacatttgcaaggattattttagggggatgctcagaatttcgctttaatgataatcttgtgagtcgttctgtttacattgcagagttggaaaagataggcataatagtcaaagcacaaaattgtttggtttttcagggaactgtagagtcaatttcagtgaagaaacccaaagaaaggacccagttttttgaggaaatcagcacttcaggagagcttataggagaatatgaagaaaagaaaagaaagttacaaaaagccgaagaggatgcacagtttaactttaataagaaaaaaaatatagcggcagagcgcagacaagcaaaattagagaaggaagaggcagaacgttaccagagtctccttgaagaactgaaaatgaacaagatacaactgcagctttttcaactataccataatgagaaaaagattcatctcctgaacaccaagttagagcatgtgaatagggatttgagtgtcaaaagagagtctttgtctcatcatgaaaacatagttaaagccaggaaaaaggaacatggaatgctaactagacaactacaacaaacagaaaaagaattaaaatcggttgaaacccttttaaatcagaagaggcctcagtacattaaagccaaagaaaacacttctcaccaccttaagaaattagatgtggctaagaaatcaataaaggacagcgaaaaacaatgttctaaacaggaagatgatataaaagccctggagacagagctggctgatttagatgctgcatggagaagttttgaaaagcagattgaggaagaaattttacataaaaagcgagacattgaactggaagccagtcagctggatcgttataaagaacttaaggaacaagtaagaaagaaagtagctacaatgactcaacaactggaaaaactgcagtgggaacagaagacagatgaagaaagactggcatttgaaaagaggaggcatggagaagttcagggaaatctaaaacaaataaaagaacaaatagaagatcataaaaaacgaatagagaagttagaggagtatacaaagacatgcatggattgcttgaaagagaaaaaacagcaagaggaaaccctagtggatgaaattgaaaaaacaaaatcaagaatgtctgaatttaatgaagaattgaatcttattagaagtgaattgcagaatgctgggattgatacccatgagggaaaacgtcagcaaaagagagcagaggttctggaacaccttaaaagactgtacccagattctgtgtttggaagactatttgacctgtgtcatcctattcataagaaataccagctggctgttactaaggtttttggccggttcatcactgccattgttgtagcctctgaaaaggtagcaaaagattgtattcgatttctgaaggaggaaagagctgaacctgagacattcctcgctctagattaccttgatatcaagccaatcaatgaaagactaagggagcttaaaggctgtaaaatggtgattgatgtcataaagactcagtttcctcagctgaagaaagtgattcagtttgtgtgtggaaatggtcttgtttgtgagactatggaagaagcaaggcatattgcactcagtggacctgaaagacagaaaacagtagctcttgatggaacattatttttaaaatctggagtgatctctggagggtcaagtgacttaaaatacaaggctagatgctgggatgagaaagagttaaagaatctaagagacagacgaagccagaaaatccaagagctaaagggtttaatgaagacactccgcaaagaaacagatttgaaacaaatacagaccctgatacagggaactcaaacacgactcaaatattcacaaaatgaactagagatgattaagaagaagcaccttgttgctttttaccaggaacaatctcagttacaaagtgaactactaaatattgagtctcaatgtattatgttgagtgaaggaatcaaggaacgacaacgaagaattaaagaatttcaagaaaagatagataaggtagaagacgatatcttccaacacttctgtgaagaaattggcgtggaaaatattcgtgaatttgagaacaaacatgttaaacggcaacaagaaattgatcaaaaaaggtatttttataaaaagatgttgactcggcttaatgttcaacttgagtatagtcgcagtcaccttaagaagaaactgaataagatcaacacattaaaagaaactatccagaaaggtagtgaagatattgatcacctaaagaaggctgaagaaaactgtctgcagacagtgaatgaactcatggcaaagcagcagcaacttaaggacatacgtgtcactcagaactccagtgccgagaaagttcaaactcaaattgaagaggaacggaagaagtttctggctgttgatagggaagtggggaaattgcaaaaagaagttgtaagtattcaaacttctctggaacagaaacgattagagaagcataacttgctgcttgattgcaaagtgcaagacattgagataatccttttgtcggggtcactggatgacatcattgaagtggagatgggaactgaagcagaaagtacccaggcaacaattgatatctatgaaaaagaagaagcctttgaaatagactacagctctctaaaagaggatttgaaggctctacagtctgatcaagaaatcgaggcccaccttaggctcttattgcagcaagtagcatcccaggaagatatcttactgaaaacagcagccccaaacctacgagcactggagaacttaaagactgtcagagacaagtttcaagagtccacagatgcttttgaggccagcagaaaggaagccagactgtgtaggcaagagttcgagcaagtgaaaaaaaggagatacgatcttttcacccagtgttttgagcatgtctcaatctcaattgatcaaatctacaagaagctctgcagaaacaacagcgcccaagcatttcttagcccagagaaccctgaagaaccttacttggagggaattagctataactgtgtggccccaggcaaacggtttatgccaatggacaatttgtcagggggagaaaagtgtgtggcagccttggctctcctgtttgctgtgcacagttttcgtcctgccccattctttgttttagatgaagtggatgcagccctagacaatactaacataggcaaagtgtcaagttacatcaaagagcaaactcaagaccagtttcagatgatagtcatctccctaaaagaagagttctattccagagccgacgcgctgatcggcatctatcctgagtacgatgactgcatgttcagccgagttttgaccctagatctttctcagtatccagacactgaaggccaagaaagcagcaagagacacggagagtcccgctaggggcagtcctgcagcagtcacctgatcactgttcagttcccactctaatactcacacagctcctccacaggagacttct

SEQ ID NO: 4 - protein, 1235 aa, Homo sapiens
MAHLELLLVENFKSWRGRQVIGPFRRFTCIIGPNGSGKSNVMDALSFVMGEKIANLRVKNIQELIHGAHIGKPISSSASVKIIYVEESGEEKTFARIILGGCSEFRFNDNLVSRSVYIAELEKIGIIVKAQNCLVFQGTVESISVKKPKERTQFFEEISTSGELIGEYEEKKRKLQKAEEDAQFNFNKKKNIAAERRQAKLEKEEAERYQSLLEELKMNKIQLQLFQLYHNEKKIHLLNTKLEHVNRDLSVKRESLSHHENIVKARKKEHGMLTRQLQQTEKELKSVETLLNQKRPQYIKAKENTSHHLKKLDVAKKSIKDSEKQCSKQEDDIKALETELADLDAAWRSFEKQIEEEILHKKRDIELEASQLDRYKELKEQVRKKVATMTQQLEKLQWEQKTDEERLAFEKRRHGEVQGNLKQIKEQIEDHKKRIEKLEEYTKTCMDCLKEKKQQEETLVDEIEKTKSRMSEFNEELNLIRSELQNAGIDTHEGKRQQKRAEVLEHLKRLYPDSVFGRLFDLCHPIHKKYQLAVTKVFGRFITAIVVASEKVAKDCIRFLKEERAEPETFLALDYLDIKPINERLRELKGCKMVIDVIKTQFPQLKKVIQFVCGNGLVCETMEEARHIALSGPERQKTVALDGTLFLKSGVISGGSSDLKYKARCWDEKELKNLRDRRSQKIQELKGLMKTLRKETDLKQIQTLIQGTQTRLKYSQNELEMIKKKHLVAFYQEQSQLQSELLNIESQCIMLSEGIKERQRRIKEFQEKIDKVEDDIFQHFCEEIGVENIREFENKHVKRQQEIDQKRYFYKKMLTRLNVQLEYSRSHLKKKLNKINTLKETIQKGSEDIDHLKKAEENCLQTVNELMAKQQQLKDIRVTQNSSAEKVQTQIEEERKKFLAVDREVGKLQKEVVSIQTSLEQKRLEKHNLLLDCKVQDIEIILLSGSLDDIIEVEMGTEAESTQATIDIYEKEEAFEIDYSSLKEDLKALQSDQEIEAHLRLLLQQVASQEDILLKTAAPNLRALENLKTVRDKFQESTDAFEASRKEARLCRQEFEQVKKRRYDLFTQCFEHVSISIDQIYKKLCRNNSAQAFLSPENPEEPYLEGISYNCVAPGKRFMPMDNLSGGEKCVAALALLFAVHSFRPAPFFVLDEVDAALDNTNIGKVSSYIKEQTQDQFQMIVISLKEEFYSRADALIGIYPEYDDCMFSRVLTLDLSQYPDTEGQESSKRHGESR

SEQ ID NO: 5 - DNA, 1406 bp, Homo sapiens
cctctgcggcgtcactgggagcccgacggaaaactgcgctaaaggcttgtctttcccctgcccgaccgaaggagccgaccttgcctgcgctacagcttccttattttcgtcgcctgttctcctgatcctgcgtgttctaaaaaccccttaggctttccatgggttcccagaccatggcggtggcgctgcccagggacttgcggcaggacgccaacctggcaaagaggaggcacgcggagctgtgcaggcagaagcgggtcttcaacgccagaaacaggataattgggggagacactgaagcctgggatgttcaagttcatgaccagaagataaaagaagctactgaaaaagctagacatgaaacctttgctgctgaaatgaggcaaaatgacaaaatcatgtgcatattggaaaaccggaaaaagagggataggaaaaatctctgtagggctatcaatgacttccaacagagctttcagaagccagaaactcgccgtgaatttgatctgtccgaccccctagcccttaagaaagatcttccagcccggcagtcagataatgatgttcggaatacgatatcaggaatgcagaaattcatgggagaggatttaaacttccatgagaggaagaaattccaagaggaacaaaacagagaatggtctttgcagcagcaaagggaatggaagaacgcccgtgctgaacaaaaatgcgcagaggccctctacacagagacaaggctgcagtttgacgagacagccaagcacctccagaagctggaaagcaccaccagaaaggcagtttgtgcatctgtgaaagacttcaacaagagccaggccatcgagtcagtggaaaggaaaaagcaagagaaaaagcaagaacaagaggacaacttggccgagatcaccaacctcctgcgtggggacctgctctccgagaacccgcagcaggcagccagctccttcgggccccaccgcgtggtccctgaccgctggaagggcatgacccaggagcagctggagcagatccgcctagtccagaagcagcaaatccaggagaagctgaggctccaggaagaaaagcgccagcgagacctggactgggaccggcggaggattcagggggctcgcgccaccctgctgtttgagcggcagcagtggcggcggcagcgcgacctgcgcagagctctggacagcagcaacctcagcctggccaaggagcagcatttgcagaaaaaatatatgaatgaagtctatacaaatcaacccacgggagactatttcacacaatttaatacaggaagtcgataatgaggaacacacccttgttcccgtcattcacgtataaagagtggctaccttaaaaaaaaaaaaaaaaaaaaaaaaaaaaaaaaaaaaaaaaaaaaaaaa

SEQ ID NO: 6 - protein, 309 aa, Homo sapiens
MRQNDKIMCILENRKKRDRKNLCRAINDFQQSFQKPETRREFDLSDPLALKKDLPARQSDNDVRNTISGMQKFMGEDLNFHERKKFQEEQNREWSLQQQREWKNARAEQKCAEALYTETRLQFDETAKHLQKLESTTRKAVCASVKDFNKSQAIESVERKKQEKKQEQEDNLAEITNLLRGDLLSENPQQAASSFGPHRVVPDRWKGMTQEQLEQIRLVQKQQIQEKLRLQEEKRQRDLDWDRRRIQGARATLLFERQQWRRQRDLRRALDSSNLSLAKEQHLQKKYMNEVYTNQPTGDYFTQFNTGSR

SEQ ID NO: 7 - DNA, 1654 bp, Mus musculus
taattggatccctgctccgccgcggaaagcggaccgccgggacagacttgctctcaaacttttgtgggcgctgcttctcggcgctgtagctttgcttatttctgtgggcttttcttgccgtgttttttttttccggatagctcctgaagcccagccgagtcccagagccatggaggtagcgatgtctaaggacctacaacaggaggccaacctagccaagaagcggtacattgacctgtgcaggcagggacggatcttcgacgccaggaacaggatcattgggggagacacacaagcctgggattttcaagtccgtgaccagaagataaaagaaataactgacaaagctaggcatgaagcctttgctgctgaaatgaagcacaatgacaaagtcatgtgcatggcgcatgaccgggaacagaggcacaggaaacagctgtgtagagctatcaatgacttccagcagaactttcagaagccagaaactcgacgtgagtttgatctttctgaccccctggccctccagaaagagcttccagcccgcatttcagacaatgacatgcggaacaccatatcaggaatgcagaagttcatgggagaggatttaaacttccaagagaggaggaggttccaaaaggaacagagcagagaatggtttctgcagcagcatggggaacgggagaaagcccgggctgaccacctactggcagaacacctccacactcagacgagactcaagtttgatgaaacagccagagagttgatgaagctggaaggctccaccaggaaggaagtctgcgcagccgtgaaagcgttcaacaagaatcaggttgtggagttgacagaaagaaagaggcaagagaagcaacaagaacaagaagacaacatgaccgagatcaccaacctgctgcatggagacctgctttctgagaaccctcgaccggtggccagctcctttgggtctcaccgtgtggtccttgaccgctggaagggcatgaaccgagagcagctggaggagatctggttcacgcagaagcggcaaatccaggagaagctgaggcttcaggaggaagagcgccagcacagcatggactgggatttgcgcaggatccggaaggctcatgccagcctgctgcatgagcggcagcagcagcgcttgcttcgggagcagcgcagggccctggactgcagcaacctcaacctggccaggcagcagtacttacagaaaaaacaaatgaatacagcctcctcaagtcagcccacagaagactatttctcacagtttaatacaaggagccgctgagagaaccgactgctttccttgtttatagtagttcctctaggagtcacgcttatttcaaagtgaatctgctccttggtctgaacaccccgtatgggcatgtgcacatgcggtcaggctctgctctgggaagagctctaacatggctttgggtgctgtggacataggacacagtgaaccattccagagaggctacactgaggcctgtgtgctgctcaagctccaagctggctgctcagccattctctgactcagtgactgaccatcataggtgttgcttcgtgtgccccatgaagcacttcttcctcagcttctcacccaccataaagaggcaccaacctgcttaccactccc

SEQ ID NO: 8 - protein, 309 aa, Mus musculus
MKHNDKVMCMAHDREQRHRKQLCRAINDFQQNFQKPETRREFDLSDPLALQKELPARISDNDMRNTISGMQKFMGEDLNFQERRRFQKEQSREWFLQQHGEREKARADHLLAEHLHTQTRLKFDETARELMKLEGSTRKEVCAAVKAFNKNQVVELTERKRQEKQQEQEDNMTEITNLLHGDLLSENPRPVASSFGSHRVVLDRWKGMNREQLEEIWFTQKRQIQEKLRLQEEERQHSMDWDLRRIRKAHASLLHERQQQRLLREQRRALDCSNLNLARQQYLQKKQMNTASSSQPTEDYFSQFNTRSR

SEQ ID NO: 9 - DNA, 336 bp, Mus musculus
  misc_feature (108)..(108): n is a, c, g, or t
  misc_feature (159)..(159): n is a, c, g, or t
  misc_feature (178)..(178): n is a, c, g, or t
  misc_feature (295)..(295): n is a, c, g, or t
  misc_feature (333)..(333): n is a, c, g, or t
gacattgtgatgtcacagtctccatcctccctggctgtgtcagcaggagagaaggtcactatgagctgcaaatccagtcagagtctgctcaacagtagaacccgaaanaactacttgtcttggtaccagcagaaaccaggtcagtctcctaaactgctnatctactgggcatccactngggaatctggggtccctgatcgcttcacaggcagtggatctgggacagatttcactctcaccatcagcagtgtgcagactgaagacctggcagtttattactgcaagcaatcttatnatctcttcacgttcggctcggggacaaagttggaaatnaaa

SEQ ID NO: 10 - DNA, 29 bp, Artificial sequence
  Synthetic primer
ttttattaatcacggcaagaaaagcccac

SEQ ID NO: 11 - DNA, 25 bp, Artificial sequence
  Synthetic primer
ttttaccggtgcctcagccgcttcc

SEQ ID NO: 12 - DNA, 278 bp, Mus musculus
agaaaagcccacagaaataagcaaagctacagcgccgagaagcagcgcccacaaaagtttgagagcaagtctgtcccggcggtccgctttccgcagcggagcagggatccaattccccgggcaacgccaacctccgttgtaagcaacggcgcctcgctcgctctccttcccccgcgccagtctcgcgagacttcgaaaagaatttcttcccgcgcttttttttttttttttcctcacgggagcacgaggaagcggctgaggcacggcgcgcagccatg

SEQ ID NO: 13 - DNA, 267 bp, Homo sapiens
agaacaggcgacgaaaataaggaagctgtagcgcaggcaaggtcggctccttcggtcgggcaggggaaagacaagcctttagcgcagttttccgtcgggctcccagtgacgccgcagaggtacagacgctcccgctctctcggaaatgtcaacaactcgttgctaaggaacggctccgcgcttgccgcgtctcgctctcttctcgcgacacttggcgaatcccttcccgcgctttttccgcgggcgcttgataacgcgggtgaggcg